(19) United States
(12) Patent Application Publication
(10) Pub. No.: US 2023/0120666 A1
(43) Pub. Date: Apr. 20, 2023

(54) EXCIPIENTS PROVIDING STABILIZATION AND ENHANCED WATER SOLUBILIZATION AND THEIR USES

(71) Applicant: NanoSynthons LLC, Mt Pleasant, MI (US)

(72) Inventors: Donald A. Tomalia, Naples, FL (US); David M. Hedstrand, Midland, MI (US); Mayank K. Singh, Mt Pleasant, MI (US); Linda S. Nixon, Mt. Pleasant, MI (US)

(73) Assignee: NanoSynthons LLC, Mt. Pleasant, MI (US)

(21) Appl. No.: 18/082,566

(22) Filed: Dec. 15, 2022

Related U.S. Application Data
(63) Continuation-in-part of application No. PCT/US2021/037513, filed on Jun. 15, 2021.
(60) Provisional application No. 63/039,108, filed on Jun. 15, 2020.

(51) Int. Cl. A61K 47/40; A61K 47/10; A61K 47/12; A61K 47/46; A61K 9/12; A61K 9/00; A61K 47/55

(52) U.S. Cl. CPC A61K 47/40; A61K 47/10; A61K 47/12; A61K 47/46; A61K 9/12; A61K 9/0046; A61K 9/0043; A61K 47/551

(57) ABSTRACT

Four major polymeric architectures, namely: (a) linear, (b) branched, (c) hyperbranched/dendritic and (d) cross-linked polymers, when formed by reaction of multifunctional alcohols, such as sugar-based alpha-, beta- or gamma-cyclodextrins, with multi-carboxylic acids form unique polyester copolymers. These copolymers have been demonstrated to substantially enhance the water-solubility and bioavailability of water insoluble compounds for a wide variety of uses.

### FIELD OF THE INVENTION

This invention concerns soluble excipients for enhancing aqueous solubility of various insoluble or difficult to solubilize compounds. It also concerns the use of insoluble excipients, either independently or in combination with soluble excipients, to produce more extensive control over active ingredient delivery.

### BACKGROUND OF THE INVENTION

Nearly 40% of all newly discovered active drug candidates possess intrinsic lipophilic structural features that ultimately lead to failure in clinical trials largely due to poor aqueous solubility properties (Dahan, A. et al., J. Control. Release 2008, 129, 1-10; van de Waterbeemd, H. et al., J. Med. Chem. 2001, 44, 1313-1333; Lipinski, C. A. et al., Adv. Drug Del. Rev., 2001, 46, 3-26). Such a challenge also exists for important members of the hemp based cannabinoid family, a widely recognized class of natural and synthetic chemical structures known to block or remediate receptor sites associated with biological inflammation, arthritis, chronic pain, epileptic activity, anxiety, appetite, sleep disorders, hair loss (Bruni, N. et al., Molecules 2018, 23, 2478/1-25; doi:10.3390/molecules23102478) or remediate certain cancers (Lv, P. et al., Journal Drug Delivery Science and Technology 2019, 51, 337-344; Yokoo, M. et al., PlosOne 2015, 10(11), e0141946). These important receptor sites invariably reside in aqueous domains that influence normal biological function, physiology and well-being of both humans and animals. As such these receptors are largely immersed in an aqueous environment, wherein, only water soluble entities may have access and be bioavailable for correcting certain dysfunctions or delivering therapeutic benefits.

Essentially all cannabinoids, many active pharmaceutical ingredients (APIs) (i.e., steroids, flavonoids, anti-inflammatories, anti-fungal, anti-microbial, etc.) and a broad range of natural products (i.e., flavors fragrances and therapies) suffer from very poor aqueous solubility properties. These reduced solubility features substantially hamper the ability to systematically deliver these materials for desired benefits or effective therapeutic dosages. Furthermore, many cannabinoids and API's are unstable and suffer from serious photo and oxidative degradation properties upon storage in an unprotected state. More specifically, cannabinoids generally exhibit very low aqueous solubility (i.e., 0.1-10 μg/mL) (Grotenhermen, F., Clin. Pharmacokinet. 2003, 42, 327-360; Mannila, J. et al., J. Pharm. Sci., 2007, 96, 312-319) and their solutions are very susceptible to external degradation upon exposure to heat, oxygen or light (Pacifici, R. et al., Clin. Chem. Lab. Med. 2018, 56, 94-96; Liebmann, J. W. et al., J. Pharm. Pharmacol. 1979, 28, 1-7). As such, critical formulation protocols involving co-solvency, micellization (R. Winnicki, R. Peet, PCT WO 2013/009928 A1, Jan. 17, 2013), nano-emulsions (Nakano, Y. et al., Med. Cannabis Cannabinoids, 2019, 2, 35-42), micro-emulsification (i.e., use of lipid-based surfactants, emulsifying agents, or formation of inclusion complexation (i.e., cyclodextrins) (Degeeter, D. M. et al., PCT WO 2017/183011 A1, Oct. 26, 2017; Saokham, P. et al., Molecules 2018, 23, 1161), micro-encapsulation in lipid-based formulations (i.e., liposomes) (W. Kleidon, J. Kirkland, U.S. Pat. No. 10,080,736 B2, issued Sep. 25, 2018) or various nanoparticles are required (Kumari, A. et al., Colloids Surf B Biointerfaces 2010, 75, 1-8; Lawrence, M. J. et al., Adv. Drug Deliv. Rev. 2000, 45, 89-121; Allen, T. M. et al., Science 2004, 303, 1818-1822; Allen, T. M. et al., Adv. Drug Deliv. Rev., 2013, 65, 36-48.

In general, many cannabinoid solubilization strategies are associated with traditional emulsification technology (ET) (see FIG. 1). Emulsification technology relies on the use of amphiphilic surfactants that self-assemble into a variety of non-covalent supramolecular assemblies referred to as liposomes or micelles as shown in FIG. 1. These metastable supramolecular assemblies may function as non-covalent host structures for incarcerating hydrophobic guest molecules such as cannabinoids. Although some solubility issues may be resolved by these protocols, many other serious deficiencies remain due to the instability of the non-covalent liposome/micelle assemblies. More specifically, it was recently reported that hydrophobic beverage can coatings readily destabilized beverages containing emulsion encapsulated CBD (defined later in the Glossary); thereby, producing unacceptable insoluble cannabinoid deposits in the products (Staniforth, J., Food Quality & Safety 2020, August/September, 18-19). Furthermore, it has been determined recently that CBD destabilizes certain traditional emulsion systems, especially under mechanical stress conditions (Francke, N. M. et al., Molecules, 2021, 26, 1469).

A strategy for more stabilized encapsulation structures has been to use cyclodextrins (CDs). Cyclodextrins constitute a family of commercially available cyclic oligosaccharides (i.e., sugars) that are produced on a large scale by the enzymatic degradation of starch. They are 6, 7 or 8-membered macrocyclic sugars derived from multiple D-glucose units linked by α-1,4-glycosidic bonds, referred to as α, β, γ-CDs, respectively. These macrocyclic sugar structures possess discrete torus-like shapes, wherein, their small rims (0.45-0.77 nm) present reactive multiple (i.e., 6-8) primary hydroxyl groups and the larger rims (0.57-0.95 nm) possess multiple (i.e., 12-16) less reactive secondary hydroxyl moieties as illustrated in FIG. 2.

A unique property associated with CD structures is their amphiphilic character, wherein their interiors are hydrophobic (i.e., lipid attractive) and their exteriors are hydrophilic (i.e., water attractive). This unique feature allows them to form a wide range of water soluble inclusion complexes where they may function as a host for a wide range of hydrophobic (i.e., lipid-like) guest molecules, especially water insoluble active pharmaceuticals (Davis, M. E. et al. Nature Reviews/Drug Discovery, 2004, 3, 1023-1035; Saokham, P. et al., Molecules, 2018, 23, 1161). The main driving force for these supramolecular self-organizations is the “hydrophobic effect” associated with the CD interiors, wherein expulsion of high energy water occurs leading to hydrophobic host-guest stoichiometries varying from 1:1, 1:2 to 2:1.

These cyclic sugar structures are very biocompatible, do not illicit immune responses and exhibit very low toxicity in animals or humans. As such, they have attained GRAS status (i.e. Generally Regarded as Safe) and are used extensively as food processing/additives which are approved by the FDA and European Medicines Agency (EMA) as excipients for many current drug delivery protocols (Braga, S. S., Biomolecules, 2019, 9, 801). According to a recent report (Chaudhari, P. et al., Experimental Eye Research, 2019, 189, 107829) more than 46 FDA approved commercial products containing CDs are currently being marketed for human use.

Although hydrophobic guest molecules may be encapsulated directly into naked cyclodextrins, there are still serious challenges and unmet needs associated with their use as in vivo excipients. The limited water solubility of some of the parent CDs is known to impart cytotoxicity by absorption through lipophilic biological membranes. This issue still remains a concern (European Agency Report, 2017, Cyclodextrins Used as Excipients, EMA/CHMP/495747/2013, 1-16). Therefore, any surface modification designed to disrupt intrinsic CD hydrogen bonding or allows attachment of water soluble polymer components to increase water solubility (Cheng, J., et al., Bioconjugate Chem. 2003, 74, 1007-1017) will force CDs to reside extracellularly and prevent their absorption through lipophilic biological membranes, thus rendering them less cytotoxic. For example, conjugating random methylated β-CD (Me-β-CD) to hydroxyethyl starch significantly lowered the cytotoxicity of the Me-β-CD-polymer conjugate relative to its monomeric form (Markenstein, L. et. al., Beilstein J. Org. Chem. 2014, 10, 3087-3096).

Historically, CDs were first incorporated into water soluble, epichorohydrin-CD co-polymers as early as 1987 (Szeman, J. et al., J. of Inclusion Phenomena, 1987, 5, 427-31; Fenyvesi, E. J., J. of Inclusion Phenomena, 1988, 6, 537-45; Renard, E. et al., Eur. Polym. J., 1997, 33, 49-57). Although these CD functionalized polymers were observed to enhance solubilities of many traditional APIs compared to monomeric CDs, they were not actively pursued due to safety concerns related to the highly toxic epichlorohydrin co-monomer.

Based on commercial availability and the ability to form biofriendly water soluble inclusion complexes with many lipophilic structures, a limited number of cyclodextrins have been integrated into several major polymer architectures including: linear (Shown, I. et al, Supramolecular Chem., 2008, 20, 6, 573-578; Cheng, J., et al., Bioconjugate Chem. 2003, 74, 1007-1017), simple branched and star-shaped (Nafree, N. et al., Colloids and Surfaces B: Biointerfaces, 2015, 129, 30-38; Pereira G. et al., Aust. J. Chem., 2012, 65, 1145-1155) type polymers, wherein they are used in a wide range of applications such as cancer imaging, diagnostics and therapies (Davis, M. et al., Nature Reviews, 2004, 3, 1023-1035; Yao, X. et al., Prog. Polymer Sci., 2019, 93, 1-35).

In contrast, the use of cyclodextrins in water insoluble crosslinked polymer architectures, referred to as “nanosponges” is very extensive (e.g., Ahmed, R. A. et al., Drug Development & Industrial Pharmacy, 2013, 39, 1263-1272; Caldera, F. et al., Inter. J. Pharma, 2017, 531, 470-479). This activity has been largely directed toward environmental issues such as the clean-up/extraction of toxic organics/pollutants (Zhao, D. et al., J. Incl. Phenom. Macrocycl. Chem., 2009, 63, 195-20), metals (Ducoroy, L. et al., Reactive & Functional Polymers, 2008, 68, 594-600) and to a lesser extent in certain drug delivery applications (Allahyari, S. et al., Expert Opinion on Drug Delivery, 2019, 16, 467-479).

That withstanding, relatively few literature examples have been reported for CD based, water soluble polymers involving the integration of CDs into either random hyperbranched (Trotta, F. et al., Beilstein J. Org. Chem., 2014, 10, 2586-2593; Tian, W. et al., Macromolecules 2009, 42, 640-651; Tian, W. et al., Macromolecules, 2009, 42, 640-651) or dendritic architectures (Namazi, H. et al., Polymer Int., 2014, 63, 1447-1455). Random hyperbranched/dendritic polymer architectures are widely recognized as key intermediates leading to the transition from soluble finite polymeric species at the gelation boundary to insoluble infinite network systems.

Historical work by Carothers (Odian, G. Principles of Polymerization, Fourth ed., 2004, J. Wiley & Sons, Hoboken, N.J.), as well as Flory, (Flory, P., J. Am. Chem. Soc., 1941, 63 (11), 3083-90) and Stockmayer (Stochmayer, W. H., J. Chem. Phys., 1943, 11(2) 45-55) have reported critical theoretical/mathematical concepts for predicting these gelation boundaries. In traditional systems, predictions of these important gelation boundaries are usually straightforward. They are generally based on the use of suitable monomer stoichiometries systematically derived from well-defined and known reactivity parameters associated with the respective multi-functional monomers. In contrast, predicting stoichiometries/conditions for avoiding gelation/crosslinking of cyclodextrin polymers is very challenging and is further discussed later.

Secondly, the low intrinsic water solubility properties of the basic parental α-,β- and γ-cyclodextrins have led to a variety of widely recognized CD surface functionalization products including: commercial sulfonation (Captisol®, trademark of CYDEX PHARMACEUTICALS, INC), hydroxypropylation (CAVCON®, trademark of Pocono Enterprise LLC) and methylation conjugates (CAVCON®, trademark of Pocono Enterprise LLC), to mention a few. In some cases, these CD modifications have led to new commercial products with enhanced solubility features, however, may exhibit certain cytotoxicity properties. In general, these conjugations have served to disrupt certain hydrogen bonded aggregation motifs hindering accessibility to CD complexation cavities.

Clearly, a better delivery system is needed for important, poorly soluble compounds that provides one or more of: bioavailability, improved solubility, and reduces toxicity compared to native cyclodextrins; enhanced dissolution; and provides a controlled release and resistance to degradation of the carried Guest molecules.

### BRIEF SUMMARY OF THE INVENTION

This invention demonstrates that engineered materials derived from the functionalization of polyols such as nano-containers (e.g., α, β, γ-cyclodextrin-type structures and their derivatives) or their incorporation with or without other poly(hydroxylic) reagents into certain major polymeric architectures (i.e., oligomeric/polymeric: linear, branched or random hyperbranched/dendritic architectures form water soluble polymeric host compounds (PHCs). These PHCs may be used effectively as vectors/matrices for enhancing water solubility properties (i.e., Excipients), as well as providing protection against external oxidative and photolytic degradation parameters of the guest molecule. Surprisingly, it has been found that when the poly(hydroxylic) alcohol is TRIS, both the PHC and the Polymer Adduct display fluorescence, making it a biological tracer among other uses of such agents. More specifically, it has been found that water insoluble substances (such as Cannabinoids, APIs, OTC, VET, AGI, nutrients, food additives, vitamins, herbal compounds, agrochemicals, cosmetic ingredients, etc.) may be confined in the PHC's as guest molecules whereby they exhibit enhanced solubility features and substantially longer shelf lives while being protected against external degradation parameters (i.e. photolytic and oxidative).

The PHCs provide this protection and water solubility either by inclusion complexation of the guest molecules within the cyclodextrin structure or by concurrent confinement within the interior void space of random hyperbranched/dendritic polymers containing cyclodextrin moieties. These 3-dimensional polymeric host structures may be designed to contain suitable interior nano-container/void space by engineering appropriate CD interiors, CD surface chemistry, branch cell symmetries, interior compositions and branch spacers. This engineering will allow optimized controlled release, as well as bioavailability of insoluble guest molecules to aqueous targets such as membranes, circulatory systems, neurological/physiological receptor sites, tissues, organs, etc. or abiotic systems and environments.

More specifically, this invention demonstrates that the water solubility of a commercially important guest molecule such as cannabinoid, i.e. CBD, may be enhanced by 8,000 to 240,000-fold (i.e., 0.500-15.1 mg/mL), compared to CBD in water alone (i.e., 0.0000627 mg/mL) Koch, N. et al., Inter. J. Pharm., 2020, 589, 119812. Similarly, the solubility of an important anti-oxidant/anti-ageing therapeutic agent such as resveratrol has been shown to be enhanced by as much as 125,000 to 766,000-fold (i.e., 5.01-30.64 mg/mL) compared to resveratrol in water alone (i.e., 0.00004 mg/mL) (Chauhan, A. et al., US. Patent #2016/0206572 A1, Jul. 21, 2016).

While not wishing to be bound by theory, it is believed that these Guest molecules are protected and confined in the Excipient by encapsulation, hydrophobic association, van derWaals association, hydrogen bonding, ionic forces, dipolar interaction or any means that impedes their ready exchange with the aqueous environment. The association energies of the confined Guest determine the rate of its release from this Excipient. When the Guest is confined in the PHC, it is referred to herein as a Polymeric Adduct.

A logical concept for remediating these challenges would be to create water soluble, hierarchical containment structures (i.e., nanoscale domains) possessing interior void space/chemical environment suitable to attract and isolate poorly soluble, hydrophobic sub-nanoscale sized API's (i.e., guest structures) from a continuous aqueous phase. In essence this guest encapsulation event is based on specific physiochemical parameters such as hydrophobic/hydrogen or ionic bonding, van derWaal/dipole interactions, as well as complementary size and shape requirements relative to the solubilizing containment structures. Furthermore, these containment structures should be of nanoscale dimensions, have sufficient physical stability (i.e., covalent versus supramolecular) to provide adequate protection against photo/chemical guest degradation and yet allow appropriate guest release rates to assure bioavailability. These are important criteria to consider in the assessment of traditional emulsion technology versus nano inclusion complexation technology as described in FIGS. 1 and 2.

While not wishing to be bound by theory, it is believed that this increase in solubility is due to encapsulation/complexation within certain functionalized major polymeric architecture compositions (i.e., linear, branched, hyperbranched polymers/dendritic polymers and oligomers) containing α, β, or γ-cyclodextrin-type structures, as well as via associations with mixtures of these polymeric intermediates, oligomers and polymers (FIG. 3).

This invention provides a polymeric host compound comprising a tetrapolymeric compound of the formula

A_{w}BₓC_{y}D_{z} Formula (I)

wherein:
the polymer of Formula (I) is a cross-linked polymer, linear polymer, simple branched polymer, hyperbranched polymer or dendritic polymer; and
monomer A is at least one multifunctional carboxylic compound and monomers B, C and D are at least one poly(hydroxylic) alcohol that can be the same or different, wherein the molar ratio of A:B:C:D is (x+y+z)/w=0.05-4; or
monomers A and B are at least one multifunctional carboxylic compound that can be the same or different, and monomers C and D are at least one poly(hydroxylic) alcohol that can be the same or different, wherein the molar ratio of A:B:C:D is (y+z)/(w+x)=0.05-4; or
monomers A and C are at least one multifunctional carboxylic compound that can be the same or different, and monomers B and D are at least one poly(hydroxylic) alcohol that can be the same or different, wherein the molar ratio of A:B:C:D is (x+z)/(w+y)=0.05-4; or
monomers A, B and C are at least one multifunctional carboxylic compound that can be the same or different, and monomer D is at least one poly(hydroxylic) alcohol that can be the same or different, wherein the molar ratio of A:B:C:D is z/(w+x+y)=0.05-4; or
w and z must each be at least 1; and
x and y are independently either 0 or at least 1; and

provided that when x and y are both 0, then the polymer of Formula (I) is not crosslinked polymer.

In Formula (I) wherein y is 0, the polymeric host compound comprises a terpolymeric compound of the formula

A_{w}BₓD_{z} Formula (II)

wherein:
the polymer of Formula (II) is a cross-linked polymer, linear polymer, simple branched polymer, hyperbranched polymer or dendritic polymer; and
monomer A is at least one multifunctional carboxylic compound, and monomers B and D are at least one poly(hydroxylic) alcohol that can be the same or different, wherein the molar ratio of A:B:D is (x+z)/w=0.05-4; or
monomers A and B are a poly(hydroxylic) alcohol that can be the same or different, and monomer D is a multifunctional carboxylic compound, wherein the molar ratio of A:B:D is z/(w+x)=0.05-4; and
w and z must both be at least 1; and
x can be 0 or at least 1.

In Formula (I) wherein x and y are both 0 the polymeric host compound comprises a binary copolymer of the formula

A_{w}D_{z} Formula (III)

wherein:
the polymer of Formula (III) is a linear polymer, simple branched polymer, hyperbranched polymer or dendritic polymer; and
the monomer A is at least one multifunctional carboxylic compound; and
the monomer D is at least one poly(hydroxylic) alcohol; and
w and z are both at least 1; and
the molar ratio of A:D is z/w=0.05 to 4; and

provided that gel formation is minimized.

In another aspect of this invention, these Polymeric Adducts can be further combined with a different Excipient or Cyclodextrin to form Hybrid Excipients. This aspect is discussed further below.

The polymeric host compound (PHC) of Formula (I), wherein the preferred hyperbranched/dendritic oligomer or polymer is water soluble, wherein the monomers are citric acid, cyclodextrins and/or polyols (i.e., MWt.<500 Da). Polyester linkages are produced which may or may not be formed sequentially, but have advantageous properties as discussed further below.

This PHC is converted into a Polymeric Adduct when at least one encapsulated Guest molecule with water solubility enhancement from about 10 to 1,000,000-fold, preferably 1,000 to about 800,000-fold, is confined. When this Polymeric Adduct derived from a water soluble PHC and the Guest molecule is a pharmaceutical, fragrance, natural product, Cannabinoids or herbal extract, then it can be used in a formulation as a cream, ointment, spray or liquid for use as a topical, ingestible or inhalable product. When this Polymeric Adduct is derived from a water insoluble PHC and the Guest molecule is a pharmaceutical, fragrance, cannabinoids or herbal extract, then it can be used in a formulation as an aqueous suspension or dry powder for use as a topical, ingestible, or inhalable product. When the Polymeric Adduct has a PHC that is a hyperbranched polymer and the Guest molecule is an agricultural agent, then it can be used as a dispersible for crop, seed, weed or insect control. Additionally, two or more soluble or insoluble Polymer Adducts can be blended to form a stable suspension for delivery of agricultural agents, pharmaceutical (API) drugs, fragrances, natural products, cannabinoids or herbal extracts. Suitable formulations for these uses are as: an oral delivery as most are non-toxic, edible formulations such as foods, tablet, lozenge, capsule, syrup, sprays, or suspension; as a topical cream, powder, ointment, gel, paste, spray, foam, or aerosol; as ophthalmic eye drops, ophthalmic ointment or gel; as a parenteral injection administered intramuscular, intravenous, or subcutaneous; as an inhalation treatment as an aerosol for the nose, nasal powder, or nebulizer; as an otic treatment by ear drops; as a rectal suppository or enema; or as a vaginal suppository or enema for humans or animals. Many other uses can be understood by the characteristics of these Excipients and Polymeric Adducts.

### BRIEF DESCRIPTION OF THE DRAWINGS

FIG. 1 illustrates the architectures of traditional micelles and liposomes and their internalization of hydrophobic guest molecules.

FIG. 2 illustrates α-, β- and γ-cyclodextrin structures to show their formula, size and approximate volume for encapsulation.

FIG. 3 illustrates the CD-citric acid and/or polyol esterified polymer structures of this invention.

FIG. 4 illustrates the linear, branched, dendritic and cross-linked polymers and shows where gelation starts as well as soluble and insoluble Excipients that can be components themselves to form Hybrid Excipients.

FIG. 5 illustrates key processes for preparing the polymers used for Excipients I, II, III and IV.

FIG. 6 illustrates reaction scheme for synthesis of Excipients I-IV.

FIG. 7 graphically illustrates a forced ranking of solubility enhancements for 21 APIs using Excipient type III of Run #65 as a Polymeric Adduct.

FIG. 8 graphically illustrates a forced ranking of solubility enhancements for 21 APIs using Excipient type II of Run #59 as a Polymeric Adduct.

FIG. 9 graphically illustrates a forced ranking of solubility enhancements for 21 APIs using Excipient type II of Run #60 as a Polymeric Adduct.

FIG. 10 graphically illustrates a forced ranking of solubility enhancements for 21 APIs using Excipient type II of Run #61 as a Polymeric Adduct.

FIG. 11 graphically illustrates a forced ranking of solubility enhancements for 21 APIs using Excipient type II of Run #62 as a Polymeric Adduct.

FIG. 12 graphically illustrates a forced ranking of solubility enhancements for 21 APIs using Excipient type I of Run #66 as a Polymeric Adduct.

FIG. 13 graphically illustrates a forced ranking of solubility enhancements for 21 APIs using Excipient type II of Run #67 as a Polymeric Adduct.

FIG. 14 graphically illustrates a forced ranking of solubility enhancements for 21 APIs using Excipient type III of Run #118 as a Polymeric Adduct.

FIG. 15 graphically illustrates a forced ranking of solubility enhancements for 21 APIs using Excipient type III of Run #119 as a Polymeric Adduct.

FIG. 16 graphically illustrates a forced ranking of solubility enhancements for 21 APIs using Excipient type III of Run #120 as a Polymeric Adduct.

FIG. 17 graphically illustrates a forced ranking of solubility enhancements for 21 APIs using Excipient type III of Run #121 as a Polymeric Adduct.

FIG. 18 graphically illustrates a forced ranking of Excipients type I-IV of the top 25 Polymeric Adducts to show solubility enhancements for CBD as the Guest in the indicated Polymeric Adduct.

FIG. 19 graphically illustrates a forced ranking of the top 14 Polymeric Adducts and categories used to show solubility enhancements of Excipient type I-IV for resveratrol as the Guest in the indicated Polymeric Adduct.

FIG. 20 graphically illustrates the forced ranking of the top 11 Polymeric Adducts and categories used to show solubility enhancements of Excipient type I-IV for curcumin as the Guest in the indicated Polymeric Adduct.

FIG. 21 graphically shows comparative dissolution profile of Run #90 RSV Polymeric Adduct and Run #108 CBD Polymeric Adduct, each at pH 1.2 and pH 6.8;

FIG. 21A shows RSV for Run #90 at both pH values; FIG. 21B shows CBD for Run #108 at both pH values; FIG. 21C shows Run #90 RSV with Excipient #94 or #97 as a Hybrid Excipient at both pH values; FIG. 21D shows #108 CDB with Excipient #94 or #97 as a Hybrid Excipient at both pH values; FIG. 21E shows Run #90 RSV with Excipients #94 and #97 as one Hybrid Excipient at both pH values; FIG. 21F shows #108 CDB with Excipients #94 and #97 as one Hybrid Excipient at both pH values.

FIG. 22 graphically shows comparative in vitro release profiles of Run #90 RSV and Run #108 CBD in PBS (pH 7.4); FIG. 22A shows RSV #90, RSV #90 and #94 as a Hybrid Excipient, RSV #90 and #97 as a Hybrid Excipient, and RSV #90, #94 and #97 as a Hybrid Excipient; FIG. 22B shows CDB #108, CBD #108 and #94 as a Hybrid Excipient, CBD #108 and #97 as a Hybrid Excipient, and CBD #108, #94 and #97 as a Hybrid Excipient.

FIG. 23 graphically illustrates a forced ranking of solubility enhancements for 5 herbicides as active ingredients using Excipient type III of Run #168 as a Polymeric Adduct.

FIG. 24 graphically illustrates a forced ranking of solubility enhancements for 3 cannabinoids as active ingredients using Excipient type III of Run #181 as a Polymeric Adduct.

FIG. 25 graphically illustrates a forced ranking of solubility enhancements for 48 active ingredients using Excipient type III of Run #181 as a Polymeric Adduct.

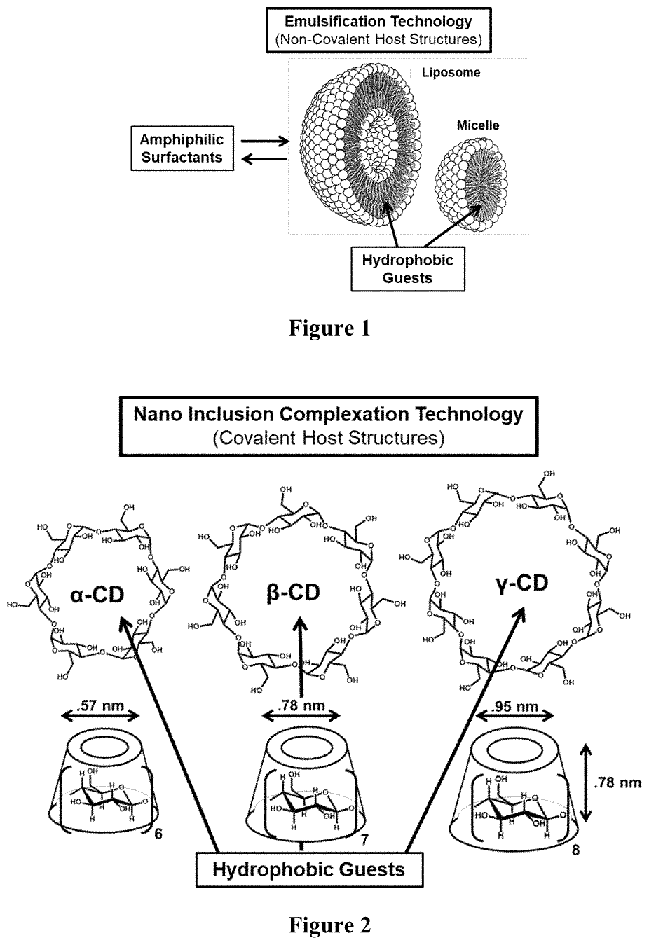

### DETAILED DESCRIPTION OF THE INVENTION

It is understood that the terminology used herein is for the purpose of describing particular embodiments only and is not intended to be limiting. As used in this specification, the singular forms “a”, “an”, and “the” include plural referents unless the content clearly indicates otherwise. The following terms in the Glossary as used in this application are to be defined as stated below and for these terms, the singular includes the plural.

Various headings are present to aid the reader, but are not the exclusive location of all aspects of that referenced subject matter and are not to be construed as limiting the location of such discussion.

Also, certain US patents and PCT published applications have been incorporated by reference. However, the text of such patents is only incorporated by reference to the extent that no conflict exists between such text and other statements set forth herein. In the event of such conflict, then any such conflicting text in such incorporated by reference US patent or PCT application is specifically not so incorporated in this patent.

### Glossary

The following terms as used in this application are to be defined as stated below and for these terms, the singular includes the plural. The bold font is not required to mean this definition but supplied to more easily find the term's meaning in this listing.
AA means adipic acid
AGI means agricultural compounds including but not limited to herbicides, fungicides, insecticides, drought tolerant chemicals, genetic modified products (GMO), agricultural seeds treatments (tablets, dustable/wettable powders, granules, suspensions, etc.), microbial and bacterial pesticides (larvicides) and others used in the agricultural industry in treatment of plants
API means hydrophobic, water insoluble or limited water solubility active pharmaceutical ingredient, whether or not it requires governmental approval to market, that is intended to treat any perceived health or wellness problem in humans or animals
Buffer/Media means Simulated Gastric Fluid (SGF pH 1.2), Phosphate Buffer (PB pH 6.8), Simulated Intestinal Fluid (SIF pH 6.8), and Phosphate Buffered Saline (PBS pH 7.4)
CA means citric acid
CA-CD-Polyol means citric acid-Cyclodextrin-polyol copolymers
CBD means a type of cannabinoid referred to as cannabidiol
CBG means a type of cannabinoid referred to as cannabigerol
CD means cyclodextrin, all forms, including but not limited to, α-, β-, γ-cyclodextrin, 2-[hydroxypropyl] β-cyclodextrin (2-HP-CD), random methylated β-cyclodextrin (Meβ-CD), sulfonated β-cyclodextrin
α-CD means a torus shaped cyclodextrin macrocycle containing six (6) glucopyranose rings possessing six (6) primary hydroxyl groups on the small rim and twelve (12) secondary hydroxyl moieties on the larger rim. (See FIG. 2.)
β-CD means a torus shaped cyclodextrin macrocycle containing seven (7) glucopyranose rings possessing seven (7) primary hydroxyl groups on the small rim and fourteen (14) secondary hydroxyl moieties on the larger rim. (See FIG. 2.)
γ-CD means a torus shaped cyclodextrin macrocycle containing eight (8) glucopyranose rings possessing eight (8) primary hydroxyl groups on the small rim and sixteen (16) secondary hydroxyl moieties on the larger rim. (See FIG. 2.)
Cannabinoids mean a wide range of substances found in the cannabis plant (e.g., cannabigerol-type (CBG), cannabigerolic acid (CBGA), cannabigerolic acid monomethylether (CBGAM), cannabigerol monomethyl ether (CBGM), cannabichromene-type (CBC), cannabichromanon (CBCN), cannabichromenic acid (CBCA), cannabi-chromevarin-type (CBCV), cannabichromevarinic acid (CBCVA), cannabidiol-type (CBD), tetrahydrocannabinol type (THC), iso-tetrahydrocannabinol-type (iso-THC), cannabinol-type (CBN), cannabinolic acid (CBNA), cannabinol methylether (CBNM), cannabinol-C₄ (CBN-C₄) cannabinol-C₂ (CBN-C₂), cannabiorcol (CBN-C₁) cannabinodiol (CBND), cannabielsoin-type (CBE), cannabielsoic acid A (CBEA-A), cannabielsoic acid B (CBEA-B), cannabicyclol-type (CBL), cannabicyclolic acid (CBLA), cannabicyclovarin (CBLV), cannabicitran-type (CBT), cannabitriol, cannabitriolvarin (CBTV), ethoxy-cannabitiolvarin (CBTVE), cannabivarin-type (CBV), cannabinodivarin (CBVD), tetra-hydrocannabivarin-type (THCV), cannabidivarin-type (CBDV), cannabigerovarin-type (CBGV), cannabigero-varinic acid (CBGVA), cannabifuran (CBF), dehydrocannabifuran (DCBF), and cannabiripsol (CBR) cannabinoids.
Cross-linked polymers mean a highly branched polymer structure, wherein, one polymer chain is linked to another polymer chain to produce bridged domains exceeding its gelation point. This polymeric architecture is usually insoluble but swells substantially in certain solvents.
CUR means curcumin
DE means degree of esterification
Dendritic polymers mean the fourth new major architectural polymer class consisting of: random hyperbranched, dendrigraft, dendron or dendrimer polymers, including rod-shaped and core-shell tecto-dendrimers as described in “Dendrimers, Dendrons, and Dendritic Polymers”, Tomalia, D. A., Christensen, J. B. and Boas, U (2012) Cambridge University Press, New York, N.Y
DI means distilled water or deionized water
EDTA means ethyl enediaminetetraacetic acid
Excipient means a polymeric host compound (PHC) of Formula (I), (II), or (III) having any degree of aqueous solubility that can include one or more of these polymeric host compounds (when more than one Excipient is used or another Cyclodextrin added then Hybrid Excipients result)
2-ETB means 2-ethoxybenzamide
FTIR analysis means Fourier-transform infrared spectroscopy and is an analytical technique used to identify organic, polymeric and inorganic materials
G means dendrimer generation, which is indicated by the number of concentric branch cell shells surrounding the dendrimer core (usually counted sequentially from the core)
GRAS means generally recognized as safe by the US Food and Drug Administration
Guest molecule means any hydrophobic or substantially water insoluble active Cannabinoids (i.e., CBD, CBG or other component from Hemp), any API, OTC, VET, AGI or any compound bonded to or encapsulated or otherwise confined by a polymer of Formula (I), (II) or (III), including but not limited to, other hydrophobic water insoluble natural products and/or materials that need protection against external chemical/photolytic degradation parameters
HPLC means high performance liquid chromatography
2-[HP]-βCD means β-cyclodextrin modified by ring opening reaction with propylene oxide to produce various degrees of ring opening product (i.e., 1-7) of 2-[hydroxypropyl]-β-cyclodextrin
Hemp means cannabis containing less than 0.3% tetrahydrocannabinol
hr. means hour(s)
Hybrid excipient means a mixture of a) a polymeric host compound with one or more of i) a second polymeric host compound, ii) a Cyclodextrin, iii) polyol, and iv) carboxylic acid
Hyperbranched polymers means highly branched three-dimensional (3D) macromolecules
Insoluble Excipient means water insoluble polymers of Formula (I), (II) or (III) such as citric acid, cyclodextrin, polyol copolymers
MeβCD means random methylated β-cyclodextrin
mg means milligram(s)
min. means minute(s)
mL means milliliter(s)
mm means millimeter(s)
mV means millivolts for magnitude of Zeta potential
MWt means molecular weight
μg means microgram(s)
μm means micrometer(s)
nm means nanometer(s)
NICT means nano-inclusion complexation technology
NIR means near infrared spectroscopy
Nanosponges or CD nanosponges means a nanoparticle consisting of cross-linked cyclodextrins able to function as a host structure for the incorporation of Guest molecules within their interior
NTA means nitrilotriacetic acid
kDa means kilodalton(s)
OTC means a broad area of products sold over the counter without a prescription or clearance by the customer (e.g., age requirement or sign a register) to purchase such product and includes, but not limited to, API, various treatments (cosmeceutical, nutraceutical, theranostics, fragrance, aromatherapy, vitamins, cosmetics, natural products, and herbal extracts), personal care (hair, skin, bath & shower, sun, oral care, sun screens, insect repellant, lubricants, ointments, salves, gels); household products (cleaning products, laundry detergents, disinfectants, antimicrobials, etc.) other similar products
PDI mean polydispersity index
PHC means a polymeric host compound A_{w}BₓC_{y}D_{z} of Formula (I), (II) or (III) and can also be used as an Excipient
Polymeric Adduct means a Guest molecule confined by a polymer of Formula (I), (II) or (III); i.e. Excipient+Guest or some PHC+Guest
PTOL means pentaerythritol
QSARs mean quantitative structure activity relationships for example solubility as a function of PHC structure
RSV means resveratrol
RT means ambient temperature, usually about 20-24° C.
Soluble Excipient means water soluble polymer of Formula (I), (II), or (III) such as citric acid, cyclodextrin, polyol copolymers
STMP means trisodium metaphosphate
SupraPlex® means the registered trademark by NanoSynthons LLC for the Excipients of this invention
TA means tartaric acid
TEG means triethylene glycol
THC means tetrahydrocannabinol
THCA means tetrahydrocannabinolic acid
THF means 3′,4′,5,7-tetrahydroxyflavone
TLC means thin layer chromatography
TRIS means tris(hydroxymethyl) aminomethane or 2-amino-2-(hydroxymethyl)propane-1,3-diol (TRIS)
μ means micron(s)
μL means microliter(s)
Ultrafiltration (UF) means membrane filtration in which hydrostatic pressure forces a liquid against a semi-permeable membrane.
UV-vis (UV) detection means the absorbance of light as the signal for measuring concentration
VET mean veterinary products including but not limited to API for animals, OTC products for animals, feeds, genetically modified chemicals (GMO), growth regulators, and others intended to be use in the animal industry
VG means vegetable glycerin or glycerin obtained from other sources

### Discussion

A variety of valuable compounds such as hemp-based cannabinoids, insoluble or hydrophobic active pharmaceutical ingredients (APIs), OTC, AGI, VET and a wide range of insoluble natural products used as agricultural products, nutrients, herbals and nutraceuticals or for therapeutic/medical purposes require an improved delivery system that can solubilize them to make them more bioavailable, stable and protected from degradation.

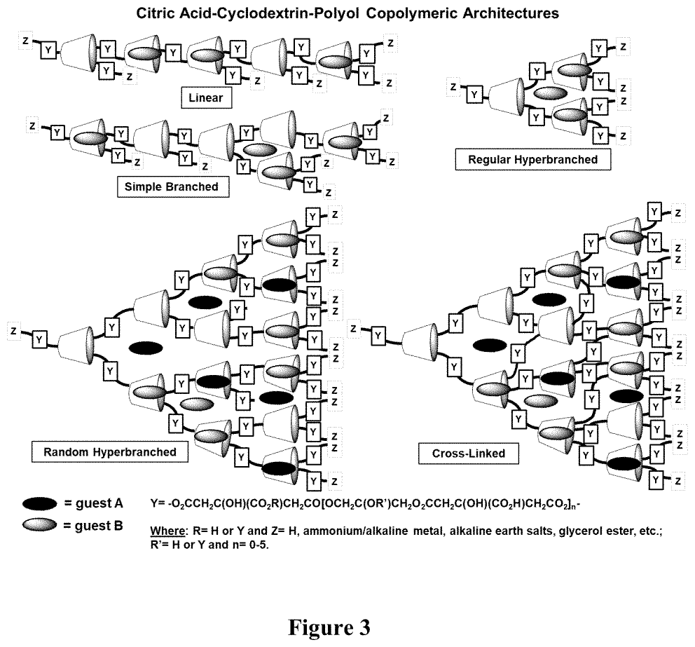

This invention provides nano-inclusion complexation technology (NICT), which avoids these instability or degradation issues by relying on stable covalent structures such as cyclodextrins (CDs) which are residing as constituents in major oligomeric intermediates or polymer architectures as described in FIG. 3.

This present invention relates to the engineered enhancement of water solubility properties associated with certain hydrophobic (i.e., water insoluble) materials including: hemp derived cannabinoids, active pharmaceutical ingredients (APIs) and OTC and natural products commonly used as herbal nutrients and medications. It has been found that water solubility properties of these water insoluble structures (i.e., guest molecules) may be substantially enhanced by concerted/confinement and/or association of guest molecules within or on the surface of α-, β-, or γ-cyclodextrins, as well as encapsulation within interior void space contained in certain polymer host structures (PHS). It is believed this solubility enhancement is based on their 3-dimensional (3D) polymer architecture, as well as their ability to minimize cyclodextrin aggregation/assembly properties.

Especially preferred oligomers or polymer architecture hosts include: (a) linear (b) random branched, and (c) hyperbranched/dendrimeric-oligomeric polymer systems. Some of these major oligomeric/polymeric architectures may possess covalently defined interior void space suitable for encapsulation of appropriately sized guest molecules, provide space filling structural features that perturb naked, monomeric cyclodextrin self-assembly events that may inhibit CD encapsulation or involve unique association with aggregate mixtures of functionalized cyclodextrins, their oligomers and polymers. This 3-D interior host space may be engineered to contain accessible and discrete interior hydrophobic cavities (i.e., α-, β- or γ-cyclodextrins, etc.) and/or space suitable for reversible guest-host site for guest complexation or association, which can be unique to the guest molecule. These architecturally driven, reversible guest-host inclusion sites for guest complexation or association to provide a wide range of unique materials that may be used for the introduction and controlled release of critical water insoluble materials into a wide variety of application options requiring enhanced water solubility properties.

As described in this invention, among the many important and unique properties exhibited by hyperbranched/dendritic architectures are the ability of these three dimensional structures to function as “host structures” in concert with the widely recognized nano-encapsulation properties of α-, β- or γ-cyclodextrins. Independently, many of these 3-D dendritic/hyperbranched host structures are widely recognized to define unique interior void space suitable for encapsulating a broad range of commercially important “guest molecules” including agrochemicals, OTC such as cosmetic ingredients and active pharmaceutical ingredients (APIs) (Tomalia, D. A. et al. Biomolecules, 2020, 642; doi:10.3390/biom10040642). As such, the present invention has combined unique architecture-based and aggregation-based hosting features of dendritic and hyperbranched oligomers/polymers with the recognized property of sugar (i.e. glucose) based cyclodextrins to form water soluble Polymeric Adducts with hydrophobic guest molecules (Guest). The hybridization of soluble macromolecular components (i.e., functionalized oligomeric linear/branched, hyperbranched/dendritic polymers) with smaller molecular (i.e., α-, β- or γ-cyclodextrin) structures has produced new compositional libraries exhibiting unexpected Guest solubilization enhancements dramatically enhanced solubility shelf lives and unique Guest protection against photo/oxidative degradation and unique controlled delivery features for administering Guest molecules and compounds such as hydrophobic cannabinoids, natural/synthetic products, as well as active pharmaceutical ingredients (APIs).

General Synthesis of Excipients:

Allowing CDs with their known properties to react with suitable co-monomers such as citric acid to form water soluble, linear, simple branched, regular/random hyperbranched/dendritic functionalized oligomers and polymers, results in compositions (i.e., copolymers/their aggregates) that benefit from the properties of both entities in an unexpected manner.

By using mild (i.e. <140° C.) processing conditions, successful esterification protocols have been developed to produce water soluble, linear, simple branched, regular/random hyperbranched/dendritic functionalized polyester compositions containing covalent α-, β-, γ-cyclodextrin host structures. However, under more severe conditions (i.e., >140° C.) a predominance of insoluble, crosslinked polymeric host compounds (PHCs) are obtained as shown in FIG. 3. It should be noted that a possible new feature of these CD containing polymers is that active guest ingredients may be encapsulated either within the cyclodextrin cavities or throughout the interior void space residing in hyperbranched/dendritic structures or by association with their aggregates as shown in FIG. 3. This designed “interior void space” phenomenon involving hyperbranched polymers, has been reported for hyperbranched poly(esteramide) polymers not containing CDs (Reven, S. et al., Internat. J. Pharma, 2010, 396, 119-126).

Unfortunately, gelation predictions that may be routinely made for traditional polyol monomers is not as easily performed for esterification reactions involving α-, β- and γ-cyclodextrins and multi-functional carboxylic acids (i.e., citric acid, tartaric acid and others) and is less well defined and more unpredictable. This is largely due to the wide range of reactivity and accessibility of the various poly(hydroxylic) moieties residing on these cyclodextrin structures. For example, α-, β- and γ-cyclodextrins each possess multiples of 6, 7 and 8 primary hydroxyl groups in concert with 12, 14 and 16 secondary hydroxylic moieties, respectively. In each of these α-, β- and γ-cyclodextrin types, special steric environment (i.e., rigidity, hydrogen bonding, etc.) is associated with these varied hydroxyl moieties that further complicate the prediction of statistical reactivity and logical stoichiometries for these more complex systems.

As such, the crosslinking principles/rules for α-, β- and γ-cyclodextrin systems frequently deviate substantially from traditional examples often giving crosslinked products under a variety of unexpectedly mild, unpredictable conditions. Undoubtedly, these unique gelation trends account for the overwhelming number of literature examples referred to as crosslinked, cyclodextrin-based “nanosponges” [e.g., (Ahmed, R. Z. et al., Drug Dev. Indust. Pharma, 2013, 39(9), 1-10); (Prabhu, P. P. et al., Res. J. Pharm. and Tech., 2020, 13(7), 3536-3544); Ananya, K. V., et al., Int. J. Res. Pharm. Sci., 2020, 11(1), 1085-1096]. Consequently, the determination of conditions required to avoid crosslinking poly hydroxylic cyclodextrin systems by reaction with poly(carboxylic acids) has remained challenging. This challenge has not only involved the elucidation of important new stoichiometries between the α, β, γ-cyclodextrin systems and citric acid/other polycarboxylic acids, but also a deeper understanding of underlying parameters (i.e., critical reaction temperatures, times, stoichiometric and other process conditions) that strongly influence transition to the cross-linked gelation state. This useful information constitutes a central theme/core for the understanding of this invention.

### Specific Citric Acid-Cyclodextrin Based Excipient Conjugates and Copolymers

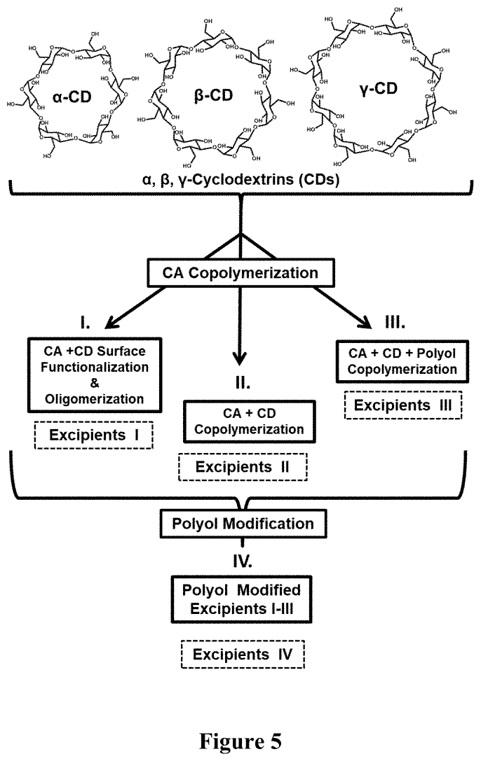

The low intrinsic water solubility properties of basic parental α-,β- and γ-cyclodextrins have prompted the development of several widely recognized CD surface functionalized commercial products including: sulfonated CDs (Captisol®, trademark of CYDEX PHARMACEUTICALS, INC), hydroxypropylated CDs (CAVCON®, trademark of Pocono Enterprise LLC) and random methylated conjugates (CAVCON®, trademark of Pocono Enterprise LLC), to mention a few. These CD modifications have led to new enhanced CD solubility features; however, certain cytotoxicity issues have continued to remain a concern (European Agency Report, 2017, Cyclodextrins Used as Excipients, EMA/CHMP/495747/2013, 1-16). Generally, these conjugations have involved the disruption of certain hydrogen bonded aggregation motifs that have hindered accessibility to CD complexation cavities. Similarly, in this invention, improvements have been developed for enhancing CD solubility/encapsulation complexation properties, as well as providing photo/chemical protection by performing a variety of unique surface modifications and co-polymerizations to produce lower toxicity water soluble, CA-CD and CA-CD-polyol co-oligomeric/copolymers/conjugates, as well as their polyol modified analogues. This has been accomplished by utilizing two key process protocols, namely;
1.) CA Copolymerizations: Citric acid esterification of α, β, γ-cyclodextrins with or without polyols to produce Excipients I-III (see FIG. 5)
2.) Polyol Modifications: Post reaction of Excipients I-III with low MWt polyols, especially glycerol, to produce Excipient IV (see FIG. 5)

These two key process protocols are used to produce all four new water soluble, CA-CD-based Excipient categories, namely, (I) citric acid functionalized-CD oligomers and citric acid-CD-polyol co-oligomers, (II) citric acid-CD copolymers, (III) citric acid-CD-polyol copolymers and (IV) polyol modified product versions of Excipient categories, as described in FIG. 5 and described more specifically in Example 17 and Table I (Runs #1-194).

The CA copolymerization protocol utilizes traditional catalyzed esterification conditions (i.e., inorganic phosphoric acid salts or strong Bronsted acids) involving the removal of water produced by esterification at 80-140° C./10-50 mm (i.e., microwave assisted or conduction heating) using tangential air flow or reduced pressure with reaction times of 1-8 hr. The “degree of esterification” (DE) is determined by monitoring the weight of water produced during the esterification reaction. In general, lower DE values of 1-3 lead to Category I type Excipients, whereas, moderate to higher DE values of 4-30, produce Category II type (i.e., contains no non-CD polyols) and Category III type (i.e., contains no non-CD polyols) Excipients. Attempting to obtain higher DE's (i.e., >10) during the preparation of Category II type Excipients often led to the formation of substantial amounts of cross-linked, water insoluble CA-CD copolymers. Quite surprisingly, while synthesizing Category III type Excipients such crosslinking at higher DE values was substantially subdued in the presence of polyols.

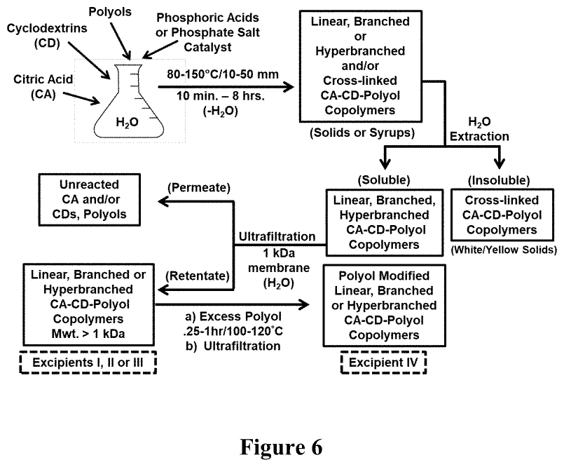

The general scheme for synthesizing Excipients I-IV involves either the phosphate catalyzed esterification of citric acid with α-, β- or γ-cyclodextrins or in the presence of a polyol to produce copolymers, as described in FIGS. 5-6. Critical reaction parameters such as reaction times, temperatures, pressures, degree of esterification and stoichiometries (see Table I) determine the nature and quality of the products produced.

Enhanced hydrophobic guest solubility and photo/chemical stabilization properties were discovered while evaluating an extensive combinatorial library of well over 120 unique CA-CD and CA-CD-polyol polyester compositions. These compositions were obtained by using the four strategies (I-IV) outlined in FIGS. 5-6. These Excipient compositions I-IV where obtained according to general synthetic protocol described in FIG. 6, using parameters and conditions described in Table I below.

Using mild/moderate reaction conditions (i.e., <140° C., shorter heating cycles, etc.) and appropriate stoichiometries (Table 1), water soluble; linear, simple branched and hyperbranched/dendritic functionalized oligomers and polymer architectures (FIGS. 3, 4 and 5) may be formed nearly exclusively. These products are referred to as: citric acid-cyclodextrin (CA-CD) or citric acid-cyclodextrin-polyol (CA-CD-polyol) copolymers (Excipients I, II and III). For example, a CA-CD-polyol copolymer (Excipient type III) synthesized from citric acid, β-cyclodextrin and glycerin (Table 1; Run #65) was obtained as a white solid, exhibiting a typical molecular weight distribution of <1 kDa to about 10 kDa. The MWt characterization is described later.

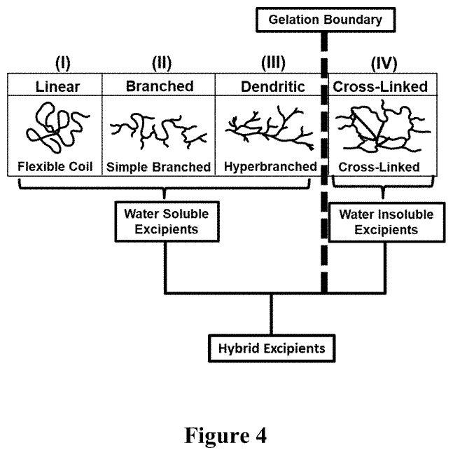

Under more severe reaction conditions, (i.e., >140° C., using longer heating cycles or inappropriate stoichiometries, etc.) a predominance of cross-linked, insoluble polymers will be formed. These products are observed as white-yellow solids upon adding water to the crude products as described in FIG. 6. These crosslinked products are referred to extensively in the literature as “nanosponges” and are not the focus of this invention. These crosslinked nanosponges form largely due to the accessibility of many intrinsic primary/secondary hydroxyl groups residing on the naked, unmodified α-, β- and γ-cyclodextrins which may esterify beyond the gelation boundary (see FIG. 4) to yield insoluble, crosslinked products.

It is interesting to note that, although 2-(hydroxypropyl) β-CD [2-HPβCD] contains a predominance of secondary hydroxyl groups, it still exhibits a high reactivity and a propensity to form crosslinked nanosponge products with citric acid. On the other hand, randomly methylated β-CD's (Me β-CD's) are an exception. Although they contained largely secondary alcohols, they form predominately linear or slightly branched oligomers with citric acid, presumably due to the limited number of secondary hydroxyl groups available for esterification after methylation which precludes crosslinking.

Finally, Excipients IV were readily obtained by post reaction of Excipients I, II, or III, bearing surface carboxylated moieties, with a variety of polyols, especially glycerin under mild/moderate conditions (i.e., 120° C./0.5 hr.) as described in FIG. 6.

It should be noted, that a portion of this invention describes various combinations of soluble, linear, branched and hyperbranched citric acid-cyclodextrin (CA-CD) and citric acid-cyclodextrin-polyol (CA-CD-polyol) copolymers with their insoluble (crosslinked) nanosponge analogues as the compositional basis for a new category of Hybrid Excipient which will be described later.

The polymeric host compounds (PHCs) are made by reaction of certain poly(carboxylic acids) or their anhydrides with poly(hydroxylic) alcohols such as α, β or γ-cyclodextrins (CDs) to form ester/polyester containing PHCs. The poly(carboxylic acids) include, but are not limited to, citric acid, itaconic, tartaric, malic, maleic, succinic, or aconitic acids, and others. These poly(carboxylic acids) may be used in molar stoichiometric ratios of 12:1 with poly(hydroxylic) CDs, however, a ratio between 3-7:12 is generally preferred. Two or more independently functionalized CDs or one or more other non-CD poly(hydroxylic) alcohols may be used in the formation of these unique polymer host structures (PHSs).

In addition to α-, β- or γ-CD's, other multifunctional poly(hydroxylic) compounds may be used in the synthesis of these proposed soluble linear, branched, hyperbranched or dendric polymers. These non-CD based poly(hydroxylic) alcohols may be introduced as spacers to improve accessibility to interior sites for enhanced CD inclusion complexation or as branched or hydrophobic/hydrophilic constituents to create additional interior hydrophobic space or peripheral hydrophilic moieties for enhanced Guest loading, respectively. These poly(hydroxylic) alcohols may include but not be limited by representative examples such as: α, β or γ-CDs, glycerol, propylene glycol, sorbitol, glucose, glucosamine, tris-(hydroxymethyl)aminomethane (TRIS), hydroxy terminated poly(ethylene glycols) (PEGs), hydroxy terminated poly(propylene glycols), pentaerythritols, and others.

Dehydration catalysts to facilitate esterification leading to desired soluble linear, random branched, hyperbranched and dendritic oligomer/polymer formation may include but are not limited to: p-toluene sulfonic acid, acidic ion exchange resins, zinc acetate, titanium tetra-butoxides, strong inorganic acids such as H₃PO₄, H₂SO₄ or inorganic phosphate salts including their inorganic salts. Most preferred are inorganic phosphate salts.

In the process, the polycarboxylic acid molecule and the multi-hydroxyl compound are reacted in the presence of a catalyst to form ester linkages resulting in a PHC with linear, random branched, hyperbranched or dendritic structures (FIG. 3).

In general, when using CD, the 2 and 6 positions are the most reactive, however, the other hydroxyl groups can be made to also react in the presence of a catalyst (i.e., phosphoric acid or inorganic phosphate salts) in an aqueous or polar solvent. The CD must have at least 2 appended carboxylate groups selected from carboxylic acid, ester, or activated ester. The mixture is heated from about 10 min. to about 8 hr. at about 80 to about 150° C. at 10-50 mm to form ester linkages. The PHC formed consists of linear, branched, cross-linked, hyperbranched dendritic polymer with a consistency from a solid to a syrup. The mixture is extracted with water to yield soluble linear, branched, hyperbranched/dendritic copolymers or insoluble cross-linked copolymers insoluble as solids. The aqueous soluble reaction mixture is subjected to ultrafiltration using a 1 kDa membrane to separate larger structures such as the hyperbranched copolymer with a molecular weight >1 kDa from smaller compounds having molecular weights <1 kDa.

The Guest molecule is added to the isolated hyperbranched copolymer having a >1 kDa size by adding the Guest molecule (optionally with a solubilizing agent like methanol or ethanol) to the PHC in water and sonicated, sometimes sonicated more than once. The PHC-Guest complex mixture is then centrifuged to remove undissolved Guest components and the supernatants combined to give the desired PHC-Guest product, Polymeric Adduct. Alternatively, the Guest molecule can be added to the copolymerization reaction mixture in the presence of the catalyst such that the PHC-Guest or a Polymeric Adduct is formed in situ.

Enhanced hydrophobic guest solubility and photo/chemical stabilization properties were discovered while evaluating a combinatorial library of well over 120 unique CA-CD and CA-CD-polyol poly(ester) compositions. These compositions were obtained by using the four strategies (I-IV) outlined in FIG. 5. These Excipient compositions I-IV were obtained according to general synthetic protocol described in FIG. 6, using parameters and conditions described in Table I below.

As described in this invention, unique and critical benefits obtained by conjugating or copolymerizing CDs with a multifunctional carboxylic acid, such as citric acid, either with or without poly(hydroxyl) agents (i.e., glycerol, d-sorbitol, pentaerythritol, etc.). These critical modifications have not only addressed the parental CD toxicity issue described above, but have also provided a broad and versatile strategy for synthesizing and engineering new cost-effective categories of excipients based on GRAS certified reactants and processes. These present Excipients have exhibited a wide range of beneficial properties. They have exhibited useful commercial applications for delivering a long list of hydrophobic APIs including, but not limited to: cannabinoids, flavonoids, steroids, anti-inflammatory agents, ocular drugs, natural products, vitamins, flavors to mention a few. This occurs by enhancing water solubility, providing photo/chemical stabilization/protection, reducing excipient cytotoxicity relative to parental cyclodextrins and allowing the systematic engineering of GRAS certified reactants to produce large combinatorial libraries of new excipient categories suitable for use as GRAS listed drug delivery vectors, for many applications such as food additives, medical therapies, nutraceuticals, fragrances, and other compounds/products.

It should be noted, that a portion of this invention describes various combinations of soluble, linear, branched and hyperbranched citric acid-cyclodextrin (CA-CD) and citric acid-cyclodextrin-polyol (CA-CD-polyol) copolymers with their insoluble (crosslinked) analogues as the compositional basis for a new category of Hybrid Excipients which will be described later in Examples 23 and 25.

### Systematic Engineering of SupraPlex® Critical Reaction Parameters to Obtain Optimized Excipient Performance Properties; Table I

Table 1 contains 194 reaction runs designed to examine the production of Excipients and Polymeric Adducts under a wide range of reaction conditions. The objective of this investigation was to determine the scope/limitations of these reactions, their resulting compositions, as well as providing a basis for comparing and quantitating respective Excipient performance levels when combined with active pharmaceutical ingredients (APIs). These critical reaction parameters, listed on the horizontal axis of Table I, were varied as a function of the Run # and included; 1) CA, CD and polyol reactant compositions, 2) phosphate catalyst type (C*), 3) stoichiometry of reactants, 4) degree of esterification (DE) and 5) weight yield of retentate product. Typical reaction conditions (i.e., reaction temperatures, times, etc.) and other details for synthesizing Excipients I-III are described under General Procedures.

### Quantitative Structure-Activity Relationships (QSARs)

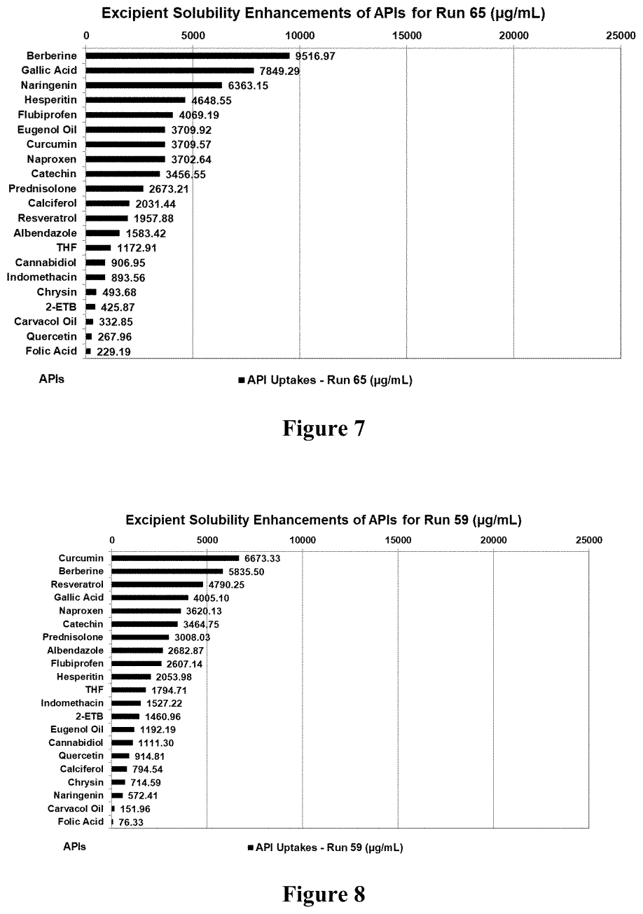

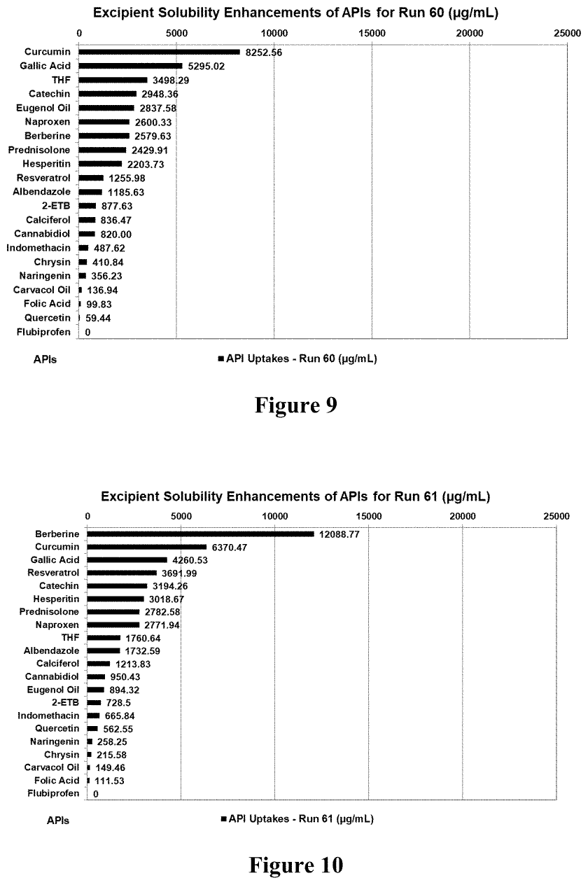

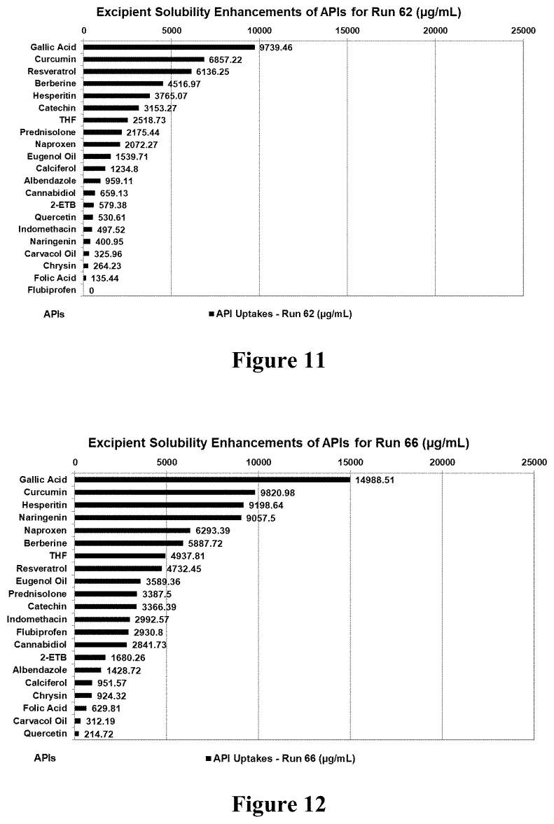

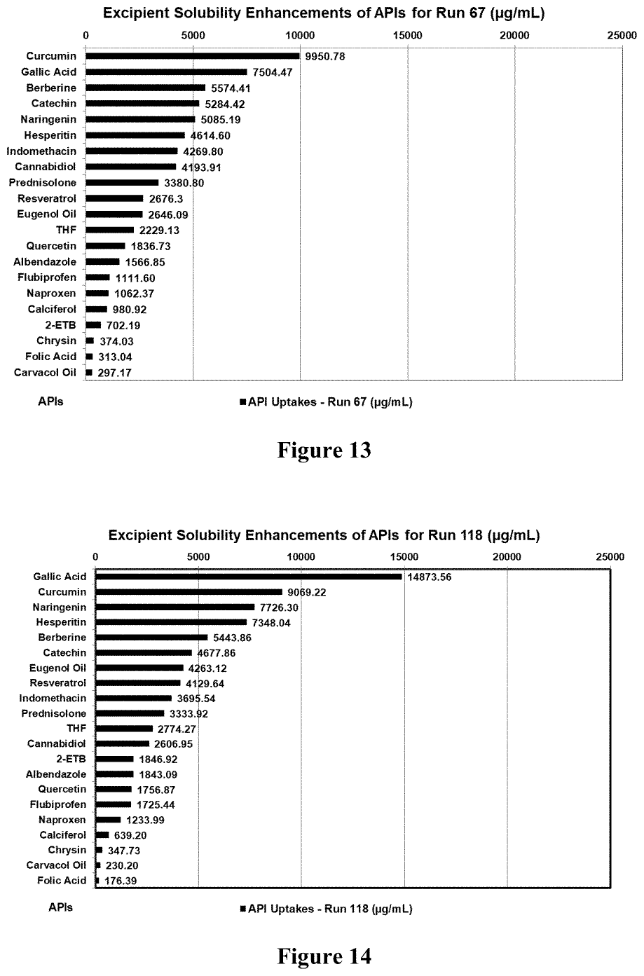

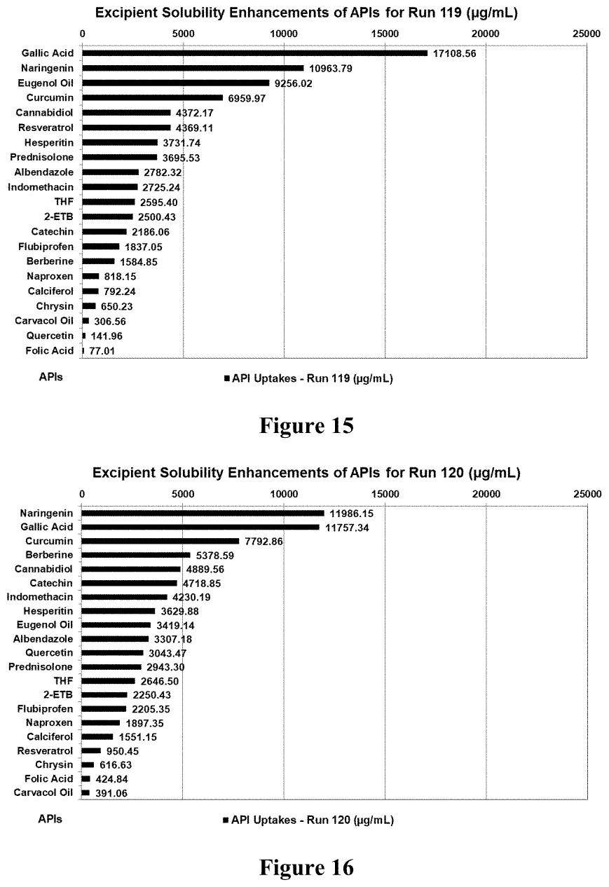

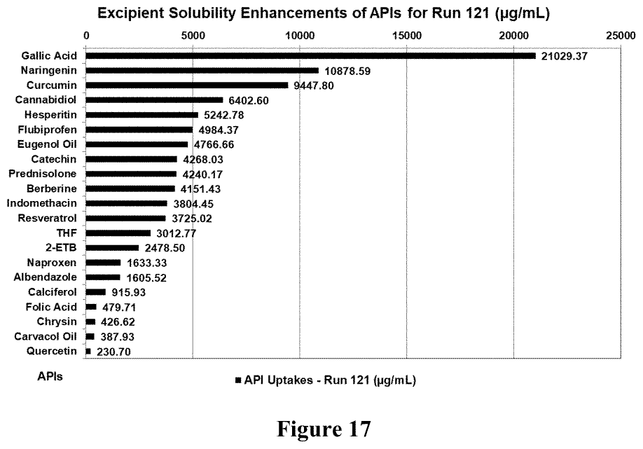

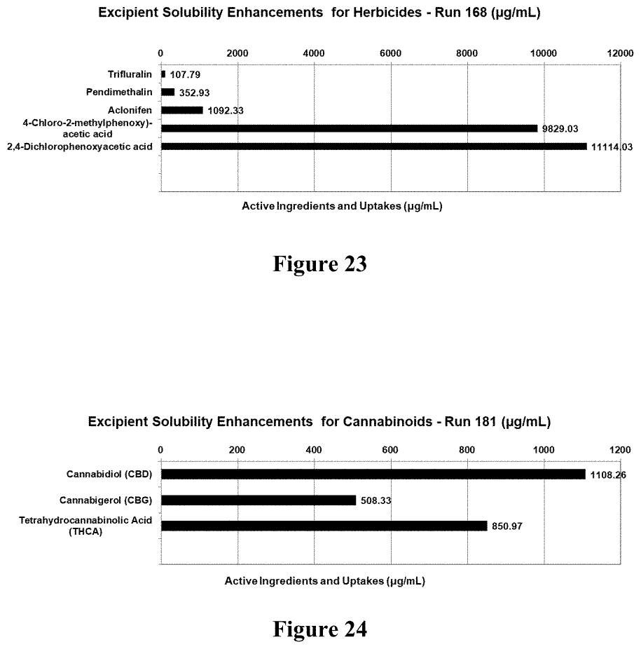

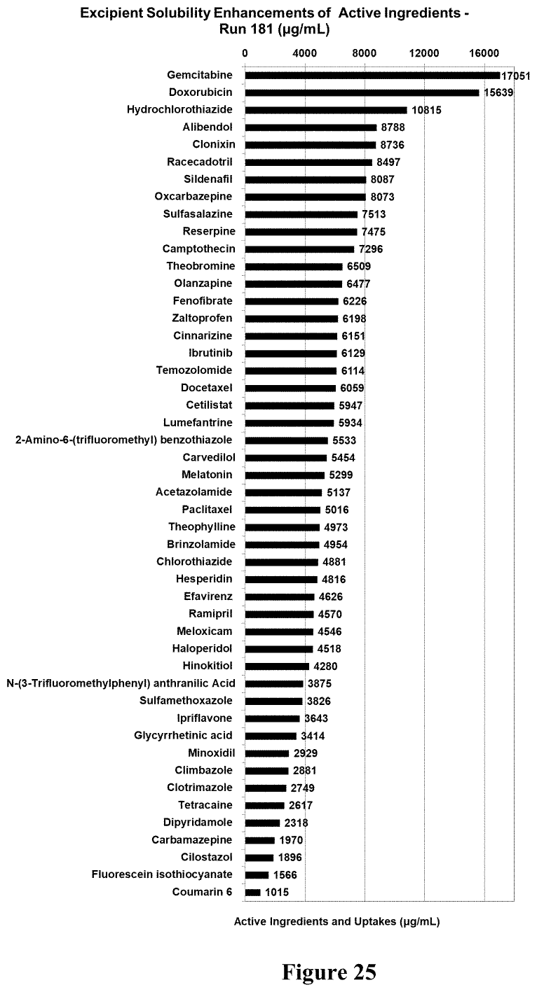

It was soon found that systematically engineering these critical reaction parameters provided discrete SupraPlex® compositions and a strategy for optimizing excipient properties required to target specific and desired APIs as a function of SupraPlex® compositions produced. These active ingredient solubility enhancement results can be understood by reviewing FIGS. 7-20, 23-25 wherein, the plots are calibrated and standardized with each other. For example, specific solubility enhancement trends/patterns are observed for various categories of active ingredients (i.e., flavonoids, steroids, anti-inflammatories, anti-oxidants, flavors, herbicides, cannabinoids etc.) This allows one to speculate on preferred “fields of use” as a function of SupraPlex® composition as discussed later.

Furthermore, these critical parameters provided guidelines for preparing specific Excipient product types I, II and III. For example, reaction temperatures/times were inextricably connected to the “degree of esterification” (DEs) observed for these various Runs 1-194; Table I. As such, synthesis runs with low DE's (i.e., 1-3) generally led to lower molecular weight type I Excipients (i.e., MWt=1-5 kDa). Typical examples in Table I would be Runs #7, #9, #21, #23, #45, #48, #56, #57, #58, #66, #77, and others.

Whereas moderate to higher DE's (i.e., (4-30) led to type II and III Excipients with molecular weights as high as 30-40 kDa. Some typical run examples in Table I would be Runs #46, #47, #87-107, #111-116, #118-121. It is interesting to note that higher DE's such as: Runs #12 (DE 21.5), #39 (DE18.3), #40 (DE 21.7), #44 (DE 11.67), #46 (DE 22.5), #52 (DE 13.03), #94 (DE 16.1), #97 (DE 54) usually were accompanied by higher levels of water insoluble, crosslinked, nanosponge type products. In fact, performing these reactions at temperatures above 140° C. (i.e., 150° C. or greater) invariably led to highly crosslinked, yellow gels or solid products with a corresponding loss of the desired water soluble Excipients I-III.

The invention will be further clarified by consideration of the following examples, which are intended to be purely exemplary of the invention.

### Materials and Methods Used in the Examples

### Materials

All chemical reagents were purchased from commercial suppliers including TCI, Sigma-Aldrich, ChemImpex, Pocono Enterprise LLC, etc.

### Equipment

Anasazi Instruments EFT-60/EM360L, NMR Spectrometer
Branson Ultrasonic Cleaner 2510R-DTH
Büchi Rotavapor R-200
Perkin Elmer 1600 Series FTIR
Hitachi U-3010 Spectrophotometer
Qsonica Q2000 Sonicator
Speedvac Plus SC110A with Thermo Savant Universal Vacuum System UVS400
VWR Model 1300U Oven
Virtis Genesis 12EL Freeze Dryer
Waters 2695 HPLC Separations Module
ZEN3600 Nano-ZS, Malvern Zetasizer
Ultrafiltration was carried out on a Millipore 1 kDa regenerated cellulose membrane in a custom tangential flow housing.

### Methods

The General Method used to determine the solubility of CBD is as follows:

CBD solubility samples were generally prepared by placing 100 mg of the solubilizing agent and 25 mg of CBD into two 4 mL vials. Water (1 mL) was added to one of the vials. Since a co-solvent was beneficial in many cases, a second vial was prepared with 1 mL of water and, usually, 0.2 mL of methanol. A third vial was prepared with 100 mg of the agent, 1 mL of water, and no CBD for use as a background standard for correcting UV-visible spectra. All three vials were processed (ultrasound) together to minimize variations.

CBD solubility was determined by UV-Visible spectrometry. Quantitation was based on a solution of CBD in methanol (100 mg/mL), which gave a k-max at 274 nm and absorbance of 0.283AU. Since all of the 1 mL samples were diluted to 10 mL to give a volume large enough for the spectrometer cuvette, a measured absorbance of 0.0283AU would correspond to 100 mg/mL of CBD in the initial 1 mL sample.

Since most of the solubility enhancing agents have their own absorbances at 274 nm, a reference or background spectrum of the agent without CBD is necessary so that its absorbance can be subtracted from the total measured to give the net value for the CBD.

Three methods were used for background subtraction:
1. For agents with very little color, the CBD is seen as a peak on the side of a peak that can be readily measured by drawing a tangent line on the interfering peak to estimate a baseline. This is usable for pure samples, such as the commercial cyclodextrins.
2. For moderately colored agents, the absorbance at 274 nm of a standard solution of the agent at the same concentration as in the mixture is subtracted from the measured absorbance of the mixture to give the net CBD absorbance.
3. For strongly colored agents, small deviations in concentration can overwhelm the CBD signal. In these cases, the full spectra are measured and the standard is multiplied by a weighting factor before subtraction. The weighting factor is adjusted to give close to a zero absorbance at many wavelengths across the difference spectrum and the CBD spectrum is what remains.

### Example 1: Water Soluble, Non-Crosslinked Citric Acid-β-Cyclodextrin Copolymers (i.e., Stoichiometry of [CA: β-CD]=[6:1])

Anhydrous citric acid (5.0 g; 0.026 mole), β-cyclodextrin (5.0 g; 0.0044 mole) and sodium dihydrogen phosphate monohydrate (1.44 g; 0.01 mole were combined with 50 mL of distilled water (DI) in a 100 mL flask to give a clear transparent solution. This aqueous mixture was reduced to a syrupy dryness on a Büchi rotavapor at 52-55° C./30 mm. Continued heating on the Büchi rotavapor at 140-150° C./11-14 mm for 20 min. produced 9.99 g of a sticky, canary yellow solid. This solid was then extracted with 3×50 mL of DI water and filtered through a Buchner funnel to yield 2.6 g of a water insoluble yellow solid product. The filtrate was then submitted to ultra-filtration (UF), using a 1 kDa membrane to produce a solid retentate (3.2 g) as the product of at least 1 kDa and a permeate weighing 2.7 g (consisting of lower molecular weight oligomeric/polymer precursor structures usually containing CA).

### UV Analysis of the Water Soluble Retentate for CBD Encapsulation

### Run #1: Cyclodextrin Product CBD Solutions

### Sample 1

The Run retentate (100 mg) was dissolved in 1 mL of water in a vial. CBD (25 mg) was added. The heterogeneous mixture was sonicated in an ultrasonic bath for 2 hr. The bath temperature rose to 40° C. during sonication. Solids were removed by centrifugation. The supernatant was decanted, the solids were resuspended in water and recentrifuged once. The combined supernatant solutions were diluted to 10.0 mL with water.

### Sample 2

The Run retentate (100 mg) was mixed with 1 mL of methanol in a vial. CBD (25 mg) was added. The heterogeneous mixture was sonicated in an ultrasonic bath for 1 hr. The bath temperature rose to 40° C. during sonication. Water (100 μL) was added to partially dissolve the retentate; the mixture was sonicated for another 1 hr. Water (4 mL) was added to precipitate excess CBD and solids were removed by centrifugation. The supernatant was decanted, the solids were resuspended in water and recentrifuged once. The combined supernatant solutions were diluted to 10.0 mL with water.

### Retentate Standard

Run 1 retentate (100 mg) was dissolved in water to give 10.0 mL of solution.

### CBD Standard

A 100 μg/mL methanol solution gave a lambda max at 274 nm with 0.287AU.
UV spectra were recorded on a Hitachi U-3010 spectrophotometer.
CBD concentration is calculated as the absorbance at 274 nm in excess of the retentate absorbance relative to the absorbance of the CBD standard. Abs means absorbance in Table 1.

TABLE 1

| Run 1 | Abs-274 | Abs-CBD | μg-CBD |
|---|---|---|---|
| retentate | 0.257 | 0 | 0 |
| ret-CBD | 0.272 | 0.015 | 52.26 |
| retCBD(MeOH) | 0.335 | 0.078 | 271.78 |
| CBD | 0.287 | | |

This result shows that CBD has an increased solubility of 2717.8-fold.

### Example 2: Non-Crosslinked, Water Soluble, Citric Acid-2-[Hydroxypropyl]-β-Cyclodextrin Copolymers (i.e. Stoichiometry of [CA:2-HP-βCD]=[8:1]) Heating cycle 1

Anhydrous citric acid (5.00 g; 0.0260 mole), 2-(hydroxypropyl)-β-cyclodextrin (5.01 g; 0.00329 mole) (i.e., degree of substitution (DS)=4.5) and sodium dihydrogen phosphate monohydrate (1.45 g; 0.0105 mole were combined with 50 mL of DI in a 100 mL round bottomed flask to give a clear transparent solution. This aqueous mixture was reduced to a clear glassy product on a Büchi rotavapor at 55° C./14 mm over 1.25 hr. Weight of the clear-white, transparent crude product was 10.97 g. This product was then heated at 110-120° C./14 mm for 20 min. to give a clear, transparent glassy syrup weighing 10.73 g which was extracted with 2×50 mL of DI exhibiting complete dissolution and no insoluble material. Ultra-filtration of this solution on a 1 kDa membrane gave a white crystalline solid retentate product weighing 4.3 g and a light yellow, glassy syrupy permeate weighing 6.3 g.

Analysis of the retentate by ¹H/¹³C-NMR, FTIR and thin layer chromatography (TLC) supported the proposed copolymeric structure.

### Run #3: Cyclodextrin Product CBD Solutions

### Sample 1

The Run retentate was dissolved in water (100 mg in 1 mL) in a vial. CBD (25 mg) was added. The heterogeneous mixture was sonicated in an ultrasonic bath for 2 hr. The bath temperature rose to 40° C. during sonication. Solids were removed by centrifugation. The supernatant was decanted, the solids were re-suspended in water and re-centrifuged. The combined supernatant solutions were diluted to 10.0 mL with water.

### Sample 2

The Run retentate was mixed with methanol (100 mg in 1 mL) in a vial. CBD (25 mg) was added. The heterogeneous mixture was sonicated in an ultrasonic bath for 1 hr. The bath temperature rose to 40° C. during sonication. Water (200 μL) was added to completely dissolve the retentate and CBD at 40° C. and the mixture was sonicated for another 1 hr. Methanol was removed in vacuo via rotavapor, the residue was resuspended in water (2 mL) and solids were removed by centrifugation. The supernatant was decanted, the solids were resuspended in water and recentrifuged. The combined supernatant solutions were diluted to 10.0 mL with water.

### Retentate Standard

Run retentate (100 mg) was dissolved in water (1 mL) and the vial was sonicated with the other samples for 2 hr. The sample was diluted with water to give 10.0 mL of solution.

CBD standard
A 100 μg/mL methanol solution gave a lambda max at 274 nm with 0.287AU.
UV spectra were recorded on a Hitachi U-3010 spectrophotometer.
CBD concentration is calculated as the absorbance at 274 nm in excess of the retentate absorbance relative to the absorbance of the CBD standard. In Table 2 Abs means absorbance.

TABLE 2

| Run 3 | Abs-274 | Abs-CBD | μg-CBD |
|---|---|---|---|
| retentate | 0.093 | 0 | 0 |
| ret-CBD | 0.121 | 0.028 | 97.56 |
| ret-CBD-MeOH | 0.168 | 0.075 | 261.32 |
| CBD | 0.287 | | |

This result shows that CBD has an increased solubility of 2673.2-fold.

### Example 3: Non-Crosslinked, Water Soluble, Citric Acid-2-[Hydroxypropyl]-β-Cyclodextrin Copolymers (i.e., Stoichiometry of [CA:2-HPβ-CD]=[8:1])

Anhydrous citric acid (5.00 g; 0.0260 mole), 2-(hydroxypropyl)β-cyclodextrin (5.07 g; 0.00329 mole) (i.e., degree of substitution (DS)=4.5) and sodium dihydrogen phosphate monohydrate (1.45 g; 0.0105 mole) were combined with 50 mL of DI water in a 100 mL round bottomed flask to give a clear transparent solution. This aqueous mixture was reduced to a clear glassy product on a Büchi rotavapor at 68-70° C./14 mm over 1 hr. Weight of the clear-white transparent crude product was 11.27 g. This reaction product was extracted with 50 mL of DI water to give virtually no insoluble material. This solution was subjected to ultra-filtration on a 1 kDa membrane to yield a beautiful white solid retentate weighing 3.38 g and a yellow syrup-like permeate weighing 7.9 g.

Analysis of the retentate by ¹H/¹³C-NMR, FTIR and thin layer chromatography (TLC) supported the proposed co-polymeric structure.

### Sample 1

The Run retentate was dissolved in water (100 mg in 1 mL) in a vial. CBD (25 mg) was added. The heterogeneous mixture was sonicated in an ultrasonic bath for 1 hr. The bath temperature rose to 40° C. during sonication. Solids were removed by centrifugation. The supernatant was decanted; the solids were resuspended in water and recentrifuged. The combined supernatant solutions were diluted to 10.0 mL with water.

### Sample 2

The Run retentate was mixed with methanol (100 mg in 1 mL) in a vial. CBD (25 mg) and water (200 μL) were added. The homogeneous mixture was sonicated in an ultrasonic bath for 1 hr. The bath temperature rose to 40° C. during sonication. Methanol was removed in vacuo via rotavapor; the residue was re-suspended in water (2 mL) and solids were removed by centrifugation. The supernatant was decanted, the solids were re-suspended in water and re-centrifuged. The combined supernatant solutions were diluted to 10.0 mL with water.

### Retentate Standard

The Run retentate (100 mg) was dissolved in water (1 mL) and the vial was sonicated with the other samples for 1 hr. The sample was diluted with water to give 10.0 mL of solution.

### CBD Standard

A 100 ug/mL methanol solution gave a lambda max at 274 nm with 0.287AU.
UV spectra were recorded on a Hitachi U-3010 spectrophotometer.
CBD concentration is calculated as the absorbance at 274 nm in excess of the retentate absorbance relative to the absorbance of the CBD standard via spectra subtraction. In Table 3 Abs means absorbance.

TABLE 3

| Run #4 | Retentate multiplier | Abs-CBD | μg-CBD |
|---|---|---|---|
| ret-CBD | 1 | 0.125 | 435.54 |
| ret-CBD-MeOH | 1 | 0.139 | 484.32 |
| CBD | 0.287 | | |

This result shows that CBD has an increased solubility of 4843.2-fold.

### Example 4: Non-Crosslinked, Water Soluble, Citric Acid-2-[Hydroxypropyl]-β-Cyclodextrin Copolymers (i.e. [CA: 2-HP-β-CD] Stoichiometry=[8:1]) Heating Cycle 2

Anhydrous citric acid (10.0 g; 0.052 mole), 2-(hydroxypropyl)-β-cyclodextrin (10.0 g; 0.00648 mole) (i.e., degree of substitution (DS)=4.5) and sodium dihydrogen phosphate monohydrate (2.48 g; 0.0181 mole) were combined with 50 mL of DI in a 200 mL flask to give a clear transparent solution. This aqueous mixture was reduced to a white solid on a Büchi rotavapor at 68-70° C./20 mm over 1 hr. Weight of the clear-white transparent crude product was 21.73 g. This reaction product was held at 68-70° C./20 mm for 2 hr. and then heated at 135-145° C./20 mm for 15 min. This reaction mixture exhibited some frothing as it became a light canary yellow color after 5 min. and then finally medium yellow under these conditions. The crude product (20.28 g.) was extracted with 2×50 mL of DI to give 3.52 g of an insoluble yellow solid after filtration. The filtrate was submitted to ultra-filtration (UF) on a 1 kDa membrane giving a light yellow solid retentate (10.0 g) and a light-yellow syrup (4.90 g) as a permeate.

Analysis of the retentate by ¹H/¹³C-NMR, FTIR, thin layer chromatography (TLC) and dynamic light scattering (DLS) supported the proposed copolymeric structure

### Run #19: Cyclodextrin Product CBD Solutions

### Sample 1

The Run #19 retentate was dissolved in water (500 mg in 1 mL) in a vial. CBD (25 mg) was added. The heterogeneous mixture was sonicated in an ultrasonic bath for 1 hr. The bath temperature rose to 40° C. during sonication. Solids were removed by centrifugation. The supernatant was decanted; the solids were resuspended in water and recentrifuged. The combined supernatant solutions were diluted to 10.0 mL with water.

### Retentate Standard

Run retentate (500 mg) was dissolved in water (1 mL). The sample was diluted with water to give 10.0 mL of solution.

CBD standard
A 100 μg/mL methanol solution gave a lambda max at 274 nm with 0.287AU.
UV spectra were recorded on a Hitachi U-3010 spectrophotometer.
CBD concentration is calculated as the absorbance at 274 nm in excess of the retentate absorbance relative to the absorbance of the CBD standard.
The absorbance of the retentate standard was stronger than the CBD containing solutions, suggesting that part of the cyclodextrin was lost in the solid precipitate. Therefore, partial spectrum subtraction was used to give a flat baseline and allow the CBD peak to be measured. In Table 4 Abs means absorbance.

TABLE 4

| Run #19 | Retentate multiplier | Abs-CBD | μg-CBD |
|---|---|---|---|
| ret-CBD | 0.93 | 0.133 | 463.41 |
| CBD | 0.287 | | |

### Run (67%) Cyclodextrin Product CBD Solutions

### Sample 1

The Run retentate was dissolved in water (1000 mg in 0.5 mL) in a vial (complete dissolution was achieved by sonication in an ultrasonic bath for 3 hr. with intermittent mixing on a vortex mixer followed by standing overnight). CBD (25 mg) was added. The heterogeneous mixture was sonicated in an ultrasonic bath for 3 hr. with intermittent mixing on a vortex mixer. The bath temperature rose to 40° C. during sonication. The viscous homogeneous portion was separated from undissolved CBD by pipette. The soluble portion was diluted to 10.0 mL with water.

### Retentate Standard

Run retentate (500 mg) was dissolved in water (1 mL). The sample was diluted with water to give 10.0 mL of solution.

CBD standard
A 100 μg/mL methanol solution gave a lambda max at 274 nm with 0.287AU.
UV spectra were recorded on a Hitachi U-3010 spectrophotometer.
CBD concentration is calculated as the absorbance at 274 nm in excess of the retentate absorbance relative to the absorbance of the CBD standard.
The absorbance of the retentate standard was weaker than the CBD containing solution. Therefore, a multiplier was used in the spectrum subtraction to give a zero response at 310 nm and allow the CBD peak to be measured. In Table 5 Abs means absorbance.

TABLE 5

| Run #19 | Retentate multiplier | Abs-CBD | μg-CBD |
|---|---|---|---|
| CBD | 1.21 | 0.133 | 463.41 |
| CBD std | 0.287 | | |

This result shows that CBD has an increased solubility of 4634.1-fold.

### Example 5: Cross-Linked, Water Insoluble, Citric Acid-α-Cyclodextrin Copolymeric Nanosponge

Anhydrous citric acid (4.00 g; 0.0208 mole), α-cyclodextrin (5.00 g; 0.00514 mole) and sodium dihydrogen phosphate monohydrate (1.44 g; 0.0104 mole) were combined with 50 mL of DI in a 100 mL flask to give a clear transparent solution. This aqueous mixture was reduced to a white solid on a Büchi rotavapor at 70-71° C./20 mm over 1 hr. to yield a white solid product. This reaction product was held at 140-150° C./18 mm while rotating on the Büchi rotavapor for 18 min., turning yellow after approximately 8 min. The medium yellow, brittle solid crude product (8.45 g) was extracted with 50 mL of DI to give a predominance of an insoluble yellow solid weighing 6.65 g. The yellow filtrate was reduced to dryness to give a bright yellow solid weighing 1.86 g. This product was fractionated by ultra-filtration (UF) on a 1 kDa membrane to give 0.36 g of a cream colored solid retentate (i.e., MWt.>1 kDa) and a syrupy permeate weighing 1.2 g (i.e., MWt.<1 kDa).

Analysis of the retentate by ¹H/¹³C-NMR, FTIR and thin layer chromatography (TLC) supported the proposed non-crosslinked, copolymeric structures.

### Run #12: Cyclodextrin Product CBD Solutions

### Sample 1

The Run precipitate was mixed in water (5 g in 50 mL) in a 4 oz bottle. The heterogeneous mixture was sonicated with a Qsonica Q2000 for 6 hr. at 25% amplitude to give a suspension that did not settle out upon standing overnight. The bottle was cooled in an ice bath during the procedure. A 1.0 mL aliquot was removed and 25 mg CBD was added. The heterogeneous mixture was sonicated in an ultrasonic cleaner for 2 hr. with intermittent mixing on a vortex mixer. The bath temperature rose to 40° C. during sonication. Excess solid CBD supernatant was removed with a spatula. The remainder was diluted to 10.0 mL with water.

### Background Standard

A 1.0 mL aliquot was removed from the sonicated mixture (without CBD) was sonicated beside in parallel to Sample 1. The sample was diluted with water to give 10.0 mL of solution.

### CBD Standard

A 100 μg/mL methanol solution gave a lambda max at 274 nm with 0.287AU.
UV spectra were recorded on a Hitachi U-3010 spectrophotometer.
CBD concentration is calculated as the absorbance at 274 nm in excess of the background standard absorbance relative to the absorbance of the CBD standard.
The absorbance of the background standard was stronger than the CBD containing solution. Therefore, a multiplier was used in the spectrum subtraction to give a zero response at 310 nm and allow the CBD peak to be measured. In Table 6 Abs means absorbance.

TABLE 6

| Run #12 | Background multiplier | Abs-CBD | μg-CBD |
|---|---|---|---|
| CBD | 0.97 | 0.109 | 379.79 |
| CBD std | 0.287 | | |

This result shows that CBD has an increased solubility of 3797.9-fold.

### Example 6: Comparative Solubility of CBD with (Naked) CDs by Sonication. Note: Used Only as a Comparative Example

Table 8 below shows comparative solubilities in water and aqueous Cyclodextrin solutions. The samples of the procedures follow Table 7.

TABLE 7
Water Solubility of CBD (μg/mL)

μg/mL

Physical Enhancements of CBD

| | | |
|---|---|---|
| (1) | CBD (no sonication)¹ | 0.0627 |
| (2) | CBD (low power sonication) | 0.4 |
| (3) | CBD (high power sonication) | 31 |

Cyclodextrin + CBD Enhancements

| | | |
|---|---|---|
| (4) | γ-Cyclodextrin + CBD | 7 |
| (5) | β-Cyclodextrin + CBD | 28 |
| (6) | Hydroxypropyl-β-Cyclodextrin + CBD | 251 |
| (7) | α-Cyclodextrin + CBD | 307 |

¹= N. Koch et al., Inter. J. Pharm., 2020, 589, 119812

### Sonication of CBD in DI Water

### Sample 1

CBD (1 g) was mixed with water (100 mL) in a 4 oz bottle. The heterogeneous mixture was sonicated with a Qsonica Q2000 for 1 hr. at 25% amplitude. The bottle was cooled in an ice bath during the procedure. A 1.0 mL aliquot was removed and the solids were removed by centrifugation. The supernatant was decanted, the solids were resuspended in water and recentrifuged. The combined supernatant solutions were diluted to 10.0 mL with water. The UV-Vis spectrum showed only a small amount of CBD.

### Sample 2

The remainder of the heterogeneous mixture was sonicated with a Qsonica Q2000 for 1 hr. at 100% amplitude. The bottle was cooled in an ice bath during the procedure. A 1.0 mL aliquot was removed and the solids were removed by centrifugation. The supernatant was decanted, the solids were resuspended in water and recentrifuged. The combined supernatant solutions were diluted to 10.0 mL with water.

CBD standard
A 100 μg/mL methanol solution gave a lambda max at 274 nm with 0.287AU.
UV spectra were recorded on a Hitachi U-3010 spectrophotometer.
CBD concentration is calculated as the absorbance at 274 nm in excess of a water blank absorbance relative to the absorbance of the CBD standard.

| CBD | retentate multiplier | Abs-CBD | μg-CBD |
|---|---|---|---|
| CBD | 1 | 0.009 | 31.36 |
| CBD | std | 0.287 | |

### Sonication of a CBD HP-BCD Solution

This result shows that CBD has an increased solubility of 313.6-fold using this above sonication protocol.

### Sample 1

Hydroxypropyl beta-cyclodextrin (5 g) was added to the remaining CBD (0.98 g)/water (98 mL) mixture in the 4 oz bottle from the CBD/water trial. The heterogeneous mixture was sonicated with a Qsonica Q2000 for 1 hr. at 100% amplitude. The bottle was cooled in an ice bath during the procedure. A 1.0 mL aliquot was removed and the solids were removed by centrifugation. The supernatant was decanted, the solids were resuspended in water and recentrifuged. The combined supernatant solutions were diluted to 10.0 mL with water. The UV-Vis spectrum showed the presence of only a small amount of CBD.

CBD standard
A 100 μg/mL methanol solution gave a lambda max at 274 nm with 0.287AU.
UV spectra were recorded on a Hitachi U-3010 spectrophotometer.
CBD concentration is calculated as the absorbance at 274 nm in excess of a water blank absorbance relative to the absorbance of the CBD standard. In Table 8 Abs means absorbance.

TABLE 8

| CBD-2HPBCD | Retentate multiplier | Abs-CBD | μg-CBD |
|---|---|---|---|
| CBD | 1 | 0.072 | 250.87 |
| CBD std | 0.287 | | |

This result shows that CBD has an increased solubility of 2508.7-fold in the presence of naked 2HPBCD.

### Sonication of Alpha-Cyclodextrin CBD Solutions

### Sample 1

Alpha-cyclodextrin (100 mg) and CBD (25 mg) were weighed into a vial. Water (1 mL) was added. The heterogeneous mixture was sonicated in an ultrasonic bath for 2 hr. The bath temperature rose to 40° C. during sonication. Solids were removed by centrifugation. The supernatant was decanted, the solids were re-suspended in water and re-centrifuged. The combined supernatant solutions were diluted to 10.0 mL with water.

### Sample 2

Alpha-cyclodextrin (100 mg) and CBD (25 mg) were weighed into a vial. Water (1 mL) and methanol (0.2 mL) were added. The heterogeneous mixture was sonicated in an ultrasonic cleaner for 2 hr. The bath temperature rose to 40° C. during sonication. Methanol was removed in vacuo via rotavapor, the residue was re-suspended in water (2 mL) and solids were removed by centrifugation. The supernatant was decanted, the solids were re-suspended in water and re-centrifuged. The combined supernatant solutions were diluted to 10.0 mL with water.

CBD standard
A 100 μg/mL methanol solution gave a lambda max at 274 nm with 0.287AU.
UV spectra were recorded on a Hitachi U-3010 spectrophotometer.
CBD concentration is calculated as the absorbance at 274 nm in excess the tangent line between 260 and 300 nm relative to the CBD absorbance standard. In Table 9 Abs means absorbance.

TABLE 9

| alpha-Cyclodextrin | Abs-CBD | μg-CBD |
|---|---|---|
| ACD-CBD | 0.088 | 306.62 |
| ACD-CBD-MeOH | 0.05 | 174.22 |
| CBD | 0.287 | |

This result shows that CBD has an increased solubility of 3066.2-fold in the presence of naked ACD.

### Sonication of Gamma-Cyclodextrin CBD Solutions

### Sample 1

Gamma-cyclodextrin (100 mg) and CBD (25 mg) were weighed into a vial. Water (1 mL) was added. The heterogeneous mixture was sonicated in an ultrasonic cleaner for 2 hr. The bath temperature rose to 40° C. during sonication. Solids were removed by centrifugation. The supernatant was decanted, the solids were resuspended in water and recentrifuged. The combined supernatant solutions were diluted to 10.0 mL with water.

### Sample 2

Gamma-cyclodextrin (100 mg) and CBD (25 mg) were weighed into a vial. Water (1 mL) and methanol (0.2 mL) were added. The heterogeneous mixture was sonicated in an ultrasonic bath for 2 hr. The bath temperature rose to 40° C. during sonication. Methanol was removed in vacuo via rotavapor, the residue was resuspended in water (2 mL) and solids were removed by centrifugation. The supernatant was decanted, the solids were resuspended in water and recentrifuged. The combined supernatant solutions were diluted to 10.0 mL with water.

### CBD Standard

A 100 μg/mL methanol solution gave a lambda max at 274 nm with 0.287AU.
UV spectra were recorded on a Hitachi U-3010 spectrophotometer.
CBD concentration is calculated as the absorbance at 274 nm in excess the tangent line between 260 and 300 nm relative to the CBD absorbance standard. In Table 10 Abs means absorbance.

TABLE 10

| gamma-Cyclodextrin | Abs-CBD | μg-CBD |
|---|---|---|
| GCD-CBD | 0.002 | 6.97 |
| GCD-CBD-MeOH | 0.008 | 27.87 |
| CBD | 0.287 | |

This result shows that CBD has an increased solubility of 278.7-fold in the presence of naked GCD. Comparative example.

### Example 7: Experimental Synthesis Runs for Production of Excipients (I)-(IV)

It was found that engineering certain critical parameters involved in the citric acid-CD-polyol modifications/copolymerization protocols provided a systematic strategy for optimizing excipient properties that could be uniquely targeted toward specific APIs. More specifically, these critical parameters include: (a) type of cyclodextrin, (b) use or absence of polyol, (c) stoichiometries of CDs, polyols, catalysts, etc. relative to citric acid, (d) type/amount of inorganic phosphate catalyst and (e) reaction conditions (i.e., reaction temperatures, times, pressures, heating mode, etc.). As such, unique API solubility enhancement profiles (FIG. 6) for each of the 194 entries in Table I could be generated and provide strong evidence for the value and uniqueness of this versatile Excipient system (SupraPlex® of NanoSynthons LLC).

When new batches of an excipient were prepared a new Run # was assigned to be allow monitoring of product differences and for tracking sample evaluations. Thus, Run #65 has several repeat batches which were designated as Runs #147, #167-#181, and #185-#187.

In the following Table I, citric acid was the predominant polycarboxylic acid used with additional examples of NTA, AA and TA; whereas, various α-, β-, γ-cyclodextrins, 2-[hydroxypropyl] β-cyclodextrin (2-HP-CD), random methylated β-cyclodextrin (Meβ-CD) were used as the poly(hydroxylic) alcohols under the conditions defined in Table I. These are all examples of this invention.

TABLE I
Experimental Runs for Syntheses of Excipients Type I-IV.

| Run | | | | | | Stoichiometry | | Retentate |
|---|---|---|---|---|---|---|---|---|
| # | Acid | CD#1 | CD#2 | Polyols | C* | A:B:C:D:C* | DE | Yield (g) |
| 1 | CA | B-CD | 0 | 0 | b | 6:1:0:0:3 | — | 2.70 |
| 2 | CA | B-CD | 0 | 0 | b | 6:1:0:0:3 | 14.50 | 4.90 |
| 3 | CA | 2HPBCD | 0 | 0 | b | 8:1:0:0:3 | 11.20 | 6.30 |
| 4 | CA | 2HPBCD | 0 | 0 | b | 7:1:0:0:3 | 6.30 | 3.38 |
| 5 | CA | B-CD | 0 | 0 | b | 12:1:0:0:5 | NA | 5.50 |
| 6 | CA | 2HPBCD | 0 | 0 | b | 15:1:0:0:5 | 21.60 | 5.00 |
| 7 | CA | B-CD | CBD | VG | b | 6:1:1:1:3 | 2.91 | 4.80 |
| 8 | CA | B-CD | CBD | VG | b | 7:1:.4:30:3 | NA | 2.00 |
| 9 | CA | B-CD | 0 | VG | b | 6:1:0:.73:2.6 | 2.69 | 4.80 |
| 10 | CA | TRIS | 0 | 0 | b | 1:1:0:0:2.6 | NA | 2.20 |
| 11 | CA | A-CD | 0 | 0 | b | 6:1:0:0:3 | 8.21 | 5.70 |
| 12 | CA | A-CD | 0 | 0 | b | 4:1:0:0:2 | 21.50 | 0.36 |
| 13 | CA | A-CD | 0 | 0 | b | 5:1:0:0:3 | 17.20 | 3.80 |
| 14 | CA | A-CD | 0 | 0 | b | 6:1:0:0:3 | 11.46 | 9.70 |
| 15 | CA | B-CD | 0 | 0 | b | 7:1:0:0:3 | 4.30 | 9.50 |
| 16 | CA | 2HPBCD | 0 | 0 | b | 7:1:0:0:3 | 5.12 | 6.10 |
| 17 | CA | 0 | 0 | PTOL | b | 2:0:0:1:5 | NA | 17.63 |
| 18 | CA | 0 | 0 | TRIS | b | 2:0:0:1:3 | NA | 2.50 |
| 19 | CA | 2HPBCD | 0 | 0 | b | 7:1:0:0:3 | 17.00 | 10.00 |
| 20 | CA | G-CD | 0 | 0 | b | 7:1:0:0:3 | 10.65 | 8.90 |
| 21 | CA | 2HPBCD | 0 | 0 | b | 7:1:0:0:5 | 2.70 | 19.50 |
| 22 | NTA | B-CD | 0 | 0 | b | 6:1:0:0:3 | NA | 7.03 |
| 23 | CA | B-CD | 0 | 0 | b | 24:1:0:0:10 | 0.78 | 2.48 |
| 24 | CA | 2HPBCD | 0 | 0 | b | 24:1:0:0:9 | NA | 9.80 |
| 25 | CA | 0 | 0 | 0 | b | 4.5:0:0:0:1 | 1.73 | 9.56 |
| 26 | 0 | B-CD | 0 | 0 | b | 0:1:0:0:8 | 6.50 | 0.75 |
| 27 | 0 | B-CD | 0 | 0 | d | NA | NA | NA |
| 28 | CA | 0 | 0 | PEG400 | b | 5:0:0:1:5.2 | NA | 3.72 |
| 29 | CA | B-CD | 0 | dsorbitol | b | 12:1:0:1:9.5 | 24.40 | 10.22 |
| 30 | CA | A-CD | 0 | dsorbitol | b | 12:1:1:0:9 | NA | 13.50 |
| 31 | CA | 2HPBCD | 0 | dsorbitol | b | 12:1:0:1:9.5 | 9.92 | 16.26 |
| 32 | CA | A-CD | 0 | VG | b | 12:1:0:1:9.5 | 10.90 | 13.98 |
| 33 | CA | A-CD | 0 | PTOL | b | 12:1:0:1:9.5 | 8.31 | 13.49 |
| 34 | CA | B-CD | 0 | dsorbitol | b | 12:1:0:8.9:1.2 | NA | 8.00 |
| 35 | CA | B-CD | 0 | PTOL | b | 12:1:0:1:1.2 | 25.10 | 13.80 |
| 36 | CA | 2HPBCD | 0 | PTOL | b | 12:1:0:1:1.2 | 10.30 | 19.92 |
| 37 | CA | A-CD | 0 | TEG | b | 12:1:0:1:1.2 | 13.20 | 16.22 |
| 38 | CA | B-CD | 0 | TEG | b | 12:1:0:1:1.2 | 13.60 | 12.54 |
| 39 | CA | B-CD | 0 | VG | b | 12:1:0:1:1.2 | 18.30 | 10.38 |
| 40 | CA | A-CD | 0 | PTOL | b | 12:1:0:1:1.2 | 21.70 | 14.63 |
| 41 | CA | 2HPBCD | 0 | 0 | b | 7.2:1:0:0:1.9 | 10.10 | 14.20 |
| 42 | CA | 2HPBCD | 0 | 0 | b | 7.2:1:0:0:1.9 | 9.99 | 24.36 |
| 43 | CA | A-CD | 0 | 0 | b | 8:1:0:0:1.5 | 10.10 | 15.01 |
| 44 | CA | A-CD | 0 | 0 | b | 6:1:0:0:2.5 | 11.67 | 12.12 |
| 45 | CA | 2HPBCD | 0 | 0 | b | 8:1:0:0:2.5 | 3.00 | 12.39 |
| 46 | CA | 2HPBCD | 0 | 0 | b | 8:1:0:0:2.5 | 22.50 | 4.91 |
| 47 | CA | 2HPBCD | 0 | 0 | b | 7.2:1:0:0:3 | 5.12 | 5.97 |
| 48 | CA | 2HPBCD | 0 | 0 | 0 | 7.2:1:0:0:0 | 1.80 | 10.10 |
| 49 | CA | 2HPBCD | 0 | 0 | b | 7.2:1:0:0:01 | 7.60 | 12.98 |
| 50 | CA | 2HPBCD | 0 | 0 | b | 7.2:1:0:0:01 | 7.45 | 12.00 |
| 51 | CA | 2HPBCD | 0 | 0 | b | 7.2:1:0:0:0.4 | 11.60 | 14.18 |
| 52 | CA | 2HPBCD | 0 | 0 | b | 7.2:1:0:0:0.8 | 13.03 | 2.28 |
| 53 | CA | 2HPBCD | 0 | 0 | b | 7.2:1:0:0:0.4 | 6.72 | 36.47 |
| 54 | CA | 2HPBCD | 0 | 0 | b | 7.2:1:0:0:0.4 | 7.00 | 36.89 |
| 55 | CA | 2HPBCD | 0 | 0 | b | 7.2:1:0:0:1.7 | 0.65 | 25.57 |
| 56 | CA | 2HPBCD | 0 | 0 | b | 7.2:1:0:0:3 | 1.20 | 29.96 |
| 57 | CA | 2HPBCD | 0 | 0 | b | 7.2:1:0:0:3 | 2.40 | 26.69 |
| 58 | CA | 2HPBCD | 0 | 0 | b | 7.2:1:0:0:3 | 1.30 | 36.40 |
| 59 | CA | 2HPBCD | 0 | 0 | b | 7.2:1:0:0:0.4 | 4.89 | 39.28 |
| 60 | CA | A-CD | 0 | 0 | b | 6:1:0:0:1.2 | 8.46 | 28.05 |
| 61 | CA | A-CD | 0 | 0 | b | 6:1:0:0:1.2 | 9.17 | 32.04 |
| 62 | CA | A-CD | 0 | 0 | b | 6:1:0:0:1.2 | 9.30 | 30.29 |
| 63 | CA | A-CD | 0 | VG | b | 6:1:0:1:1.2 | 8.90 | 13.69 |
| 64 | CA | A-CD | 0 | 0 | b | 4:1:0:0:2 | 7.13 | 5.71 |
| 65 | CA | B-CD | 0 | VG | b | 6:1:0:1:1.4 | 14.56 | 35.69 |
| 66 | CA | MeBCD | 0 | 0 | b | 6:1:0:0:1.4 | 2.69 | 35.83 |
| 67 | CA | MeBCD | 0 | 0 | b | 6:1:0:0:1.4 | 6.40 | 16.95 |
| 68 | CA | MeBCD | 0 | PTOL | b | 6:1:0:1:1.4 | 10.05 | 20.76 |
| 69 | CA | MeBCD | 0 | VG | b | 6:1:0:1:1.4 | 6.94 | 14.91 |
| 70 | CA | MeBCD | 0 | 0 | b | 4:1:0:0:1.4 | 6.10 | 13.07 |
| 71 | CA | MeBCD | 2HPBCD | 0 | b | 12:1:1:0:2.7 | 15.78 | 44.40 |
| 72 | CA | MeBCD | 0 | VG | b | 6:1:0:1:1.4 | 6.99 | BrokeFlask |
| 73 | CA | MeBCD | 0 | VG | b | 6:1:0:1:1.4 | 6.61 | 16.15 |
| 74 | CA | MeBCD | 0 | PTOL | c | 6:1:0:1:1.4 | 3.54 | 15.81 |
| 75 | CA | MeBCD | 0 | VG | c | 6:1:0:1:1.4 | 4.69 | 11.65 |
| 76 | CA | MeBCD | 0 | PTOL | c | 6:1:0:1:1.4 | 7.41 | 18.84 |
| 77 | CA | MeBCD | 0 | 0 | b | 4:1:0:0:1.4 | 1.47 | 8.99 |
| 78 | CA | MeBCD | 0 | dsorbitol | b | 6:1:0:1.4 | 7.79 | 18.50 |
| 79 | CA | MeBCD | 2HPBCD | 0 | b | 12:1:1:0:2.7 | 8.67 | 24.96 |
| 80 | CA | MeBCD | 2HPBCD | 0 | b | 8:1:1:0:2.7 | 6.47 | 16.14 |
| 81 | CA | MeBCD | 2HPBCD | 0 | b | 20:1:1:0:2.7 | 7.62 | 29.68 |
| 82 | CA | B-CD | 0 | 0 | 0 | 6:1:0:0:0 | — | — |
| 83 | CA | B-CD | 0 | 0 | 0 | 6:1:0:0:0 | — | — |
| 84 | CA | MeBCD | 0 | 0 | c | 4:1:0:0:1.4 | 4.29 | — |
| 85 | CA | MeBCD | 0 | VG | c | 6:1:0:1:1.3 | 5.76 | 16.15 |
| 86 | CA | B-CD | 0 | 0 | 0 | 2:1:0:0:0 | NA | 1.49 |
| 87 | CA | MeBCD | 0 | 0 | c | 4:1:0:0:1.4 | 4.38 | 12.38 |
| 88 | CA | MeBCD | 0 | VG | a | 6:1:0:1:1.3 | 7.58 | 19.01 |
| 89 | CA | MeBCD | 0 | 0 | a | 4:1:0:0:1.3 | 13.68 | 20.92 |
| 90 | CA | MeBCD | 0 | VG | a | 4:1:0:1:1.3 | 5.56 | 21.57 |
| 91 | CA | MeBCD | 0 | dsorbitol | a | 4:1:0:1:1.3 | 7.24 | 16.55 |
| 92 | CA | MeBCD | 0 | PTOL | a | 4:1:0:1:1.3 | 8.29 | 7.88 |
| 93 | CA | MeBCD | 0 | 0 | a | 6.8:1:0:0:4.4 | 28.00 | 10.52 |
| 94 | CA | 2HPBCD | 0 | 0 | a | 7.2:1:0:0:4.7 | 16.10 | 1.36 |
| 95 | CA | MeBCD | 2HPBCD | 0 | a | 14:1:1:0:1 | 36.00 | 13.38 |
| 96 | CA | MeBCD | BCD | 0 | a | 12:1:1:0:9 | 22.17 | 9.65 |
| 97 | CA | MeBCD | 2HPBCD | 0 | a | 14:1:1:0:9 | 54.00 | 2.90 |
| 98 | CA | MeBCD | 0 | VG | a | 4:1:0:1:0 | 10.14 | 20.93 |
| 99 | CA | 0 | 0 | VG | 0 | 8:0:0:1:0 | 9.70 | 13.75 |
| 100 | CA | 0 | 0 | VG | 0 | 7.6:0:0:1:0 | 9.70 | 43.09 |
| 101 | CA | 0 | 0 | dsorbitol | 0 | 6:0:0:1:0 | 4.01 | 36.98 |
| 102 | CA | 0 | 0 | PTOL | 0 | 8:0:0:1:0 | 4.09 | 3.58 |
| 103 | CA | MeBCD | 0 | VG | c | 3:1:0:1:1.5 | 4.60 | 10.10 |
| 104 | CA | MeBCD | 0 | VG | a | 3:1:0:1:3 | 7.81 | 13.90 |
| 105 | CA | MeBCD | 0 | VG | b | 3:1:0:1:1.5 | 8.87 | 16.72 |
| 106 | CA | MeBCD | 0 | dsorbitol | a | 3:1:0:1:4 | 9.55 | 17.21 |
| 107 | CA | MeBCD | 0 | VG | c | 4:1:0:1:1.4 | 8.33 | 16.12 |
| 108 | CA | MeBCD | 0 | 0 | c | 4:1:0:0:3 | NA | 9.35 |
| 109 | CA | MeBCD | 0 | 0 | c | 4:1:0:0:3 | 1.34 | 20.36 |
| 110 | CA | MeBCD | 0 | 0 | c | 4:1:0:0:3 | 2.90 | 13.27 |
| 111 | CA | MeBCD | 2HPBCD | 0 | c | 4:0.5:0.5:0:3 | 5.77 | 15.17 |
| 112 | CA | 2HPBCD | 0 | 0 | c | 6:1:0:0:2 | 4.88 | 62.67 |
| 113 | CA | 2HPBCD | 0 | 0 | c | 4:1:0:0:2 | 4.67 | 53.0 |
| 114 | CA | 2HPBCD | MeBCD | 0 | c | 4:1:0.1:2 | 7.26 | 56.78 |
| 115 | CA | 2HPBCD | MeBCD | VG | c | 4:1:1:1:2 | 7.50 | 52.84 |
| 116 | CA | 2HPBCD | 0 | VG | b | 4:1:0:1:2 | 13.20 | 47.09 |
| 117 | CA | 2HPBCD | 0 | 0 | b | 4:1:0:0:2 | NA | 17.86 |
| 118 | CA | MeBCD | 0 | VG | c | 4:1:0:1:1.7 | 3.60 | 14.52 |
| 119 | CA | MeBCD | 0 | VG | c | 4:1:0:1:1.5 | 4.40 | 30.39 |
| 120 | CA | MeBCD | 0 | VG | a | 4:1:0:1:1.5 | 6.06 | 44.90 |
| 121 | CA | MeBCD | 0 | VG | c | 4:1:0:1:1.5 | 5.84 | 40.90 |
| 122 | CA | MeBCD | 0 | VG | c | 4:1:0:1:1.5 | 4.97 | 30.17 |
| 123 | CA/TA | MeBCD | 0 | VG | c | 4:1:0:1:1.5 | 7.30 | 29.6 |
| 124 | CA | MeBCD | 0 | VG | c | 4:1:0:1:1.5 | 5.15 | 54.41 |
| 125 | CA | MeBCD | 0 | VG | c | 4:1:0:1:1.5 | 4.37 | 56.83 |
| 126 | CA | MeBCD | 0 | VG | c | 4:1:0:1:1.5 | NA | 10.26 |
| 127 | CA | MeBCD | 0 | VG | c | 4:1:0:1:1.5 | NA | NA |
| 128 | CA | MeBCD | 0 | VG | c | 4:1:0:1:1.5 | 5.15 | 57.5 |
| 129 | CA | MeBCD | 0 | VG | c | 3:1:0:1:0.4 | 5.43 | 95.6 |
| 130 | CA | MeBCD | 0 | VG | c | 3:1:0:1:0.4 | 7.68 | 93 |
| 131 | CA | MeBCD | 0 | VG | c | 4:1:0:1:1.5 | 7.48 | 61.7 |
| 132 | CA | MeBCD | 0 | VG | c | 3:1:0:1:1.4 | 3.46 | 59.7 |
| 133 | CA | MeBCD | 0 | VG | c | 2:1:0:1:1.5 | 6.03 | 29.7 |
| 134 | CA | MeBCD | 0 | VG | c | 1:1:0:1:1.5 | 3.64 | 33.2 |
| 135 | CA | MeBCD | 0 | VG | c | 2:1:0:1:1.5 | 1.12 | 27.8 |
| 136 | CA | MeBCD | 0 | VG | c | 3:1:0:1:1 | 1.08 | 31.2 |
| 137 | CA | MeBCD | 2HPBCD | VG | c | 3:1:1:1:1 | 1.45 | 47.5 |
| 138 | CA | MeBCD | 2HPBCD | VG | c | 3:0.5:1:1:3 | 3.45 | 40.5 |
| 139 | CA | MeBCD | 2HPBCD | VG | c | 3:0.5:0.5:1:3 | 3.66 | 39.7 |
| 140 | CA | MeBCD | 2HPBCD | VG | c | 3:0.25:1:1:3 | 2.08 | 16.6 |
| 141 | CA | MeBCD | 2HPBCD | VG | c | 3:0.125:1:1:3 | 5.05 | 14.42 |
| 142 | CA | MeBCD | 2HPBCD | VG | c | 3:1:1:1:1 | 4.18 | 95.8 |
| 143 | CA/TA | MeBCD | 0 | VG | c | 2:1:0:1:3 | 1.62 | 48.54 |
| 144 | CA/TA | MeBCD | 0 | VG | c | 2:1:0:1:1.8 | 4.94 | 58.42 |
| 145 | CA | 2HPBCD | 0 | 0 | c | 1:1:0:0:3 | 5.91 | 56.48 |
| 146 | CA | 2HPBCD | 0 | VG | c | 1:1:0:3:1 | 7.5 | 66.99 |
| 147 | CA | B-CD | 0 | VG | c | 6:1:0:1:1.37 | 6.64 | 84.2 |
| 148 | CA | A-CD | 0 | 0 | c | 6:1:0:0:4 | 6.31 | 44.92 |
| 149 | CA | B-CD | 0 | VG | c | 3:1:0:1:4 | 8.14 | 46.50 |
| 150 | CA | B-CD | 0 | VG | c | 1:1:0:1:4 | 10.31 | 32.90 |
| 151 | CA | B-CD | 0 | VG | c | 2:1:0:1:4 | 8.62 | 32.5 |
| 152 | CA | B-CD | 0 | VG | c | 4:1:0:6:4 | 9.74 | 42.87 |
| 153 | CA | B-CD | 0 | VG | c | 5:1:0:6:4 | 12.24 | 48.97 |
| 154 | CA | B-CD | 0 | VG | c | 6:1:0:6:4 | 6.62 | 45.40 |
| 155 | CA | B-CD | 0 | VG | c | 6:1:0:6:4 | 7.89 | — |
| 156 | CA | B-CD | 0 | VG | c | 6:1:0:6:4 | 15.04 | — |
| 157 | CA | B-CD | 0 | VG | c | 6:1:0:6:4 | 14.83 | 46.71 |
| 157 | CA | B-CD | 0 | VG | c | 6:1:0:6:4 | 14.83 | 46.71 |
| 158 | CA | B-CD | 0 | VG | c | 6:1:0:6:4 | 17.67 | — |
| 159 | CA | B-CD | 0 | VG | c | 6:1:0:4:3 | 11.83 | 48.82 |
| 160 | CA | B-CD | 0 | VG | 0 | 6:1:0:4:0 | 7.97 | 52.2 |
| 161 | CA | B-CD | 0 | VG | c | 6:1:0:6:4 | 12.47 | 43.67 |
| 162 | CA | B-CD | 0 | VG | c | 6:1:0:6:4 | 14.83 | — |
| 163 | CA | 2HPBCD | 0 | VG | c | 6:1:0:6:4 | 12.7 | — |
| 164 | CA | 2HPBCD | 0 | VG | c | 6:1:0:9:4 | 9.29 | 42.44 |
| 165 | CA | B-CD | 0 | VG | c | 6:1:0:1:1.37 | 10.41 | 44.10 |
| 166 | CA | B-CD | 0 | VG | c | 6:1:0:1:1.37 | 12.73 | 45.98 |
| 167 | CA | B-CD | 0 | VG | c | 6:1:0:1:1.37 | 7.36 | 94.94 |
| 168 | CA | B-CD | 0 | VG | c | 6:1:0:1:1:37 | 6.08 | 95.9 |
| 169 | CA | B-CD | 0 | VG | c | 6:1:0:1:1.37 | — | 51.94 |
| 170 | CA | B-CD | 0 | VG | c | 6:1:0:1:1.37 | 8.29 | 94.26 |
| 171 | CA | B-CD | 0 | VG | c | 6:1:0:1:1.37 | 14.37 | 45.26 |
| 172 | CA | B-CD | 0 | VG | c | 6:1:0:1:1.37 | 11.09 | 92.29 |
| 173 | CA | B-CD | 0 | VG | c | 6:1:0:1:1.37 | 7.97 | 94.50 |
| 174 | CA | B-CD | 0 | VG | c | 6:1:0:1:1.37 | 9.83 | 93.17 |
| 175 | CA | B-CD | 0 | VG | c | 6:1:0:1:1.37 | 9.74 | 93.25 |
| 176 | CA | B-CD | 0 | VG | c | 6:1:0:1:1.37 | 14.28 | 90.11 |
| 177 | CA | B-CD | 0 | VG | c | 6:1:0:1:1.37 | 12.70 | 91.20 |
| 178 | CA | B-CD | 0 | VG | c | 6:1:0:1:1.37 | 12.38 | 91.42 |
| 179 | CA | B-CD | 0 | VG | c | 6:1:0:1:1.37 | 11.75 | 91.86 |
| 180 | CA | B-CD | 0 | VG | c | 6:1:0:1:1.37 | 12.38 | 91.42 |
| 181 | CA | B-CD | 0 | VG | c | 6:1:0:1:1.37 | 13.1 | 90.1 |
| 182 | CA | B-CD | 0 | TRIS | c | 6:1:0:1:1 | 12.47 | 91.2 |
| 183 | CA | B-CD | 0 | TRIS/VG | c | 6:1:0:1:1 | 9.89 | 93 |
| 184 | CA | 0 | 0 | TRIS | 0 | 1:0:0:1:0 | — | 93.25 |
| 185 | CA | B-CD | 0 | VG | c | 6:1:0:1:1.37 | 11.75 | 91.86 |
| 186 | CA | B-CD | 0 | VG | c | 6:1:0:1:1.37 | — | — |
| 187 | CA | B-CD | 0 | VG | c | 6:1:0:1:1.37 | 12.39 | 91.42 |
| 188 | CA | G-CD | 0 | VG | c | 6:1:0:1:1.37 | 3.19 | 98.07 |
| 189 | CA | A-CD | 0 | VG | c | 6:1:0:1:1.37 | 12.89 | 89.66 |
| 190 | CA | G-CD | 0 | VG | c | 6:1:0:1:1.37 | 5.19 | 96.89 |
| 191 | CA | G-CD | 0 | VG | c | 6:1:0:1:1.37 | 10.02 | 93.99 |
| 192 | CA | A-CD/B-CD/ | | VG | c | 6:(0.33:0.33:0.33):1:1.37 | 7.46 | 94.80 |
| | | G-CD | | | | | | |
| 193 | CA/TA | A-CD/B-CD/ | | VG | c | 3:3:(0.33:033:0.33):1:1.37 | 6.13 | 95.73 |
| | | G-CD | | | | | | |
| 194 | CA/TA/AA | A-CD/B-CD/ | | VG | c | 2:2:2:(0.33:033:0.33):1:1.37 | 2.89 | 97.98 |
| | | G-CD | | | | | | |

C* = Catalysts: (a) NaH₂PO₂, (b) NaH₂PO₄, (c) Na₂HPO₄, (d) STMP
NA = not available
VG = vegetable glycerin

### Part A: Synthesis and Characterization of Citric Acid-Cyclodextrin Conjugates, Condensates and Copolymers

### General Syntheses (Runs #1-194)

In general, neat CDs or CD-polyol mixtures were combined with stoichiometric excesses of citric acid (i.e., 1-12 molar excess) in the presence of an inorganic phosphate salts (i.e., Na₂HPO₄, NaH₂PO₄, NaH₂PO₂, etc.), using a minimum amount of DI water to produce a homogenous reaction mixture. Physical, unbound water is then removed from the reaction mixture under reduced pressure (70-120° C./15-30 mm) or at atmospheric pressure (i.e., 3-8 hr./110-130° C.) to yield crude, white solids. These solid mixtures are then dehydrated to produce ester functionality using traditional or microwave assisted heating (i.e., below 150° C.) for varying times (i.e., 0.25 to 8 hr.) until a desired level of ester was attained. Progression of the esterification leading to CD conjugates or copolymers was monitored by FTIR, ¹³C-NMR, TLC, DLS, as well as by weight loss observed during this heating phase. Monitoring the weight of water formed during the reaction was used to estimate the “degree of esterification” (DE). At this stage, the crude white solid products are combined with suitable volumes of DI water (i.e., 50-200 mL) to determine the extent of crosslinking. The level of crosslinking is usually enhanced by heating over 150° C. This is determined by the amount of crude product remaining insoluble. Any insoluble product is removed by filtration and/or centrifuging. The soluble components are appropriately diluted with DI water and submitted to ultrafiltration on a 1 kDa. membrane where they are separated into a higher molecular weight retentate fraction and a lower molecular weight permeate fraction. These fractions are monitored by both FTIR and ¹³C-NMR, wherein characteristic ester signals are exhibited for all products over 1 kDa in the retentate and characteristic signals for lower molecular weight carboxylate reactants/products (i.e., unreacted citric acid, etc.) are observed in the permeate as described below.

### Example 8: Synthesis and Characterization of Citric Acid-Polyol Hyperbranched Copolymers (Evidence for Guest Encapsulation in Non-CD Hyperbranched Polymeric Architecture)

Part A: Table I; Run #99: Citric Acid [8.0 Mole]+Glycerol [1 Mole]→Hyperbranched Poly(Glyceride) Copolymer (Produces a 208× Fold CBD Solubility Enhancement)

Citric acid (50.69 g; 0.2640 mole) and glycerin (4.69 g; 0.344 mole) were charged into a 250 mL round-bottomed flask with 50 mL of DI water. An endothermic dissolution occurred to give a clear viscous solution upon swirling with slight heating. The physical, unbound water was removed from the reaction mixture on a Büchi rotavapor under vacuum over a period of 1 hr. The reaction mixture was heated under vacuum (i.e., 85-140° C./14 mm), followed by heating at 140° C./14 mm for 40 min, 145° C./14 mm for 50 min. and then at 150° C./14 mm for 60 min. The crude white product weighed 49.35 g, indicating a weight loss of 6.03 g (Degree of esterification=9.70). This crude product was completely soluble in DI water (3×50 mL) to give a light yellow solution, filtered through a Whatman filter paper and fractionated on a UF with a membrane cut-off of 1 kDa. The light yellow solid retentate weighed 13.75 g and the permeate (cream colored syrup) weighed 35.68 g. Characterization of the retentate by FTIR, ¹³C-NMR and TLC supported the proposed hyperbranched citric acid based poly(glyceride) product. Evaluation of this product according to UV based “solubility enhancement” protocol indicated a CBD uptake of 14.0 μg/mL; thus, representing a solubility enhancement of 208×-fold compared to unassisted naked CBD solubility in water of 0.06725 μg/mL (Koch, N. et al., Inter. J. Pharm., 2020, 589, 119812).

Part B: Table I; Run #102: Citric Acid [8.0 Mole]+Pentaerytheritol [1.0 Mole] [symbol] Hyperbranched Poly(ester) Copolymer (461× enhancement)

Citric acid (50.69 g; 0.2640 mole) and pentaerythritol (4.68 g; 0.344 mole) were charged into a 250 mL round-bottomed flask with 50 mL of DI water. The reaction mixture was placed on a Büchi rotavapor and heated for 4 hr. to remove unbound water (i.e., 25-142° C./29 mm). This gave a fluffy white solid that did not convert into a melt like the analogous reactions with glycerin and d-sorbitol. It appears to be a cross-linked product. Wt=52.86 g of a white friable solid indicating a weight loss of 2.53 g (Degree of esterification=4.09). Adding 3×50 mL of water and filtering gave a wet white solid weighing 55.82 g. This solid was dried in an oven at 70° C. to give 24.17 g of a clear flowable syrup when hot. Quite surprisingly, this product was soluble in 75 mL of water and fractionated by UF on a 1 kDa membrane to give 7.2 g of a clear glassy solid as the retentate and 5.01 g of an amber syrup as a permeate. The FTIR, ¹³C-NMR and TLC confirmed the proposed hyperbranched polyester product. Evaluation of this product according to UV based “solubility enhancement” protocol indicated a CBD uptake of 31.0 μg/mL; thus, representing a solubility enhancement of 461×-fold compared to unassisted CBD solubility in water of 0.06725 μg/mL (Koch, N. et al., Inter. J. Pharm., 2020, 589, 119812).

### Example 9: Excipient Characterization

The type of CD (i.e., α-, β- and γ-cyclodextrin) and reaction conditions used (i.e., citric acid molar excesses, reaction times and temperature/pressure) profoundly influences the amount/yield of insoluble, cross-linked product versus soluble, non-cross-linked (i.e., linear, branched, hyperbranched/dendritic poly(ester) condensate that are obtained. Cross-linked products are generally formed at more severe, higher reaction temperatures (i.e., >150° C.) and may be assessed by adding DI water to the crude reaction mixtures. Cross-linked products are obtained as gels or solids which may be isolated by filtration and/or centrifugation and oven dried at 70° C. The soluble filtrates are submitted to ultra-filtration on a 1 kDa membrane where they are separated into a retentate fraction containing higher molecular weight esterification products (i.e., MWt.>1 kDa) and a permeate fraction which contains lower molecular weight materials (i.e., catalyst, unreacted citric acid, etc.). The retentate products are reduced to dryness on a Büchi rotavapor and generally obtained as sparkling white solid products. These >1 kDa products may be further fractionated either by traditional membrane dialysis or Amicon membrane filtration; wherein, specific membrane MWt cut-off limits are used to determine molecular weight distributions.

FTIR:

Progress of the CA-CD esterification reactions is readily monitored by using FTIR. For example, (Run #118, retentate) shows citric acid carboxylate absorption bands at 1717.56 cm⁻¹ and 1636.02 cm⁻¹ which are systematically diminished as new absorption bands assigned to CD and polyol ester formation are observed to appear at 1733.87 cm⁻¹, 1158.12 cm⁻¹ and 1054.22 cm⁻¹. The characteristic carboxylic acid absorption signals do not completely disappear since citric acid excesses are generally used for syntheses of the CA-CD copolymers; thus, leading to products containing substantial amounts of carboxylic acid end groups.

Examination of Run #118, permeate by FTIR as well as by TLC confirms the presence of residual citric acid and lower molecular wt. esters (i.e., glycerides) (i.e., MWt.<1 kDa) with characteristic absorption bands at 1728.00 cm⁻¹, 1202.05 cm⁻¹ and 1059.61 cm⁻¹.

¹³C-NMR:

This ¹³C-NMR spectroscopic methodology corroborated the FTIR retentate assignments (i.e., Run #118) and confirmed the expected polyester copolymer products. Characteristic citric acid carbonyl carbon sp2 resonance bands at 173.61 ppm and 176.95 ppm as well as for sp3 carbons at 43.45 ppm and 73.47 ppm are observed early in the reaction. According to earlier reported protocols, (Mamajanov, I. et al., Orig. Life Evol. Biosp., 2015, 45, 123-137; Halpern, J. M. et.al., J. Biomed. Mater. Res. Part A, 2014, 102A, 1457-1477), progress of esterification is accompanied by transformation of these bands into sp3 resonance bands at 43.41 ppm and 73.47 ppm accompanied by formation of new sp2 carbonyl resonance bands at 170.70 ppm, 173.09 ppm and 176.32 ppm.

Dynamic Light Scattering

Evidence for the formation of PHC Adducts with RSV and CBD was obtained by using Dynamic Light Scattering (DLS) protocols. The hydrodynamic diameter and polydispersity index of Excipient type I (#109) and its complexes with RSV and CBD were determined using a dynamic light scattering instrument (ZEN3600 Nano-ZS, Malvern Zetasizer, UK) equipped with a backscattering angle of 173°.

The average particle size for a typical (naked) Excipient (Run #109) was 2.814 nm and exhibited a low polydispersity index of 0.17. As expected, complexation of this Excipient with RSV or CBD produced an increase in hydrodynamic diameters to 3.882 nm and 3.555 nm, respectively; whereas, polydispersity indices increased to 0.20 and 0.21, respectively. The change in particle size and polydispersity index was mainly due to the successful complexation of RSV and CBD.

HPLC

The analytical HPLC method was validated for estimation of:

Cannabidiol at https://www.waters.com/webassets/cms/library/docs/720006426en.pdf; Tetrahydrocannabinol at https://www.waters.com/webassets/cms/library/docs/720006426en.pdf); Resveratrol at A. Chauhan, et al., US Patent #2016/0206572 and

Ibrutinib at https://www.nveo.org/index.php/journal/article/view/4761/3858 as per earlier reports with minor modification.

These analyses were performed with Waters 2695 Separation Module (Waters, USA) equipped with a photodiode array detector (PDA-996) and Refractive Index Detector (2414) through Waters Reliant C18 analytical column (4.6×250 mm, 5 μm) at RT.

Fluorescent Excipient Quantum Yields

Quantum yields were determined by comparison to a well characterized standard (Quinine sulfate). Thus, absorption per gram of the standard was measured at 346 nm on a Hitachi U-3010 UV-Visible spectrophotometer and emission per gram of the standard was measured on a Perkin-Elmer LS-50B spectrofluorometer. The ratio of the emission to absorption, combined with the known quantum yield, 0.58 (58%), gives an “instrument constant” for the spectrophotometer/spectrofluorometer pair that includes all instrument variables, such as geometry, detector sensitivity, and electronic gain. The ratio of emission to absorption for an unknown sample, measured under the same instrumental conditions, can be multiplied by the “instrument constant” to give the quantum yield of the unknown.

Quantum yields of Run #182, #183, and #184 (a duplicate preparation of Run #18) were determined by this method and are list in the Table 11 below:

TABLE 11

| Run# | Quantum Yield % |
|---|---|
| 182 | 1.90 |
| 183 | 10.06 |
| 184 | 6.05 |

### Example 10: Blue Fluorescent Type III; SupraPlex® Excipients) Suitable for In Vitro or In Vivo Imaging

SupraPlex® Excipients (i.e., Runs #182 and #183) containing TRIS were found to exhibit a brilliant blue fluorescence (i.e., emission at approximately 425 nm) when irradiated with a UV source at 385 nm. These blue fluorescent Excipients were readily synthesized in high yield according to standard procedures used for Run #10 as well as other related Excipients described in Table I. More specific synthesis details are described in Example 11 below. These shiny, cream colored, solid fluorescent Excipients were readily soluble in water and produced brilliant blue fluorescent solutions even at relatively high dilutions. These unexpected fluorescence properties were confirmed by using a fluorescence spectrometer (Perkin Elmer, LS 50B) with excitation and emission at 385 nm and 425 nm, respectively. Their quantum yields were estimated to be similar to that observed for quinine sulphate. It was noted that aqueous solutions appeared to be quenched in the presence of certain iron salts (i.e., Fe^{+2/+3}). All spectroscopic analyses (i.e., FTIR, ¹H, and ¹³C-NMR, etc.) produced spectra data consistent with proposed poly(ester) condensate structures/compositions described in claim 1, and appeared to contain certain unidentified heterocyclic components.

SupraPlex® Excipients, Runs #182 and #183, exhibited substantial water solubility enhancement properties for CBD and resveratrol, respectively, as described in Table 12 below:

TABLE 12

| | CBD | RSV |
|---|---|---|
| Run# | (μg/mL) | (μg/mL) |
| #182 | 937.39 | 15037.15 |
| #183 | 967.82 | 15945.49 |

Additional applications and utilities for these materials were demonstrated by using the fluorescent properties of these Excipients as an imaging feature for tracking the biodistribution properties of these Excipients, as well as to monitor the various administration modes of these Excipients in animal models.

More specifically, these SupraPlex® Excipients were administrated to male SD rats (150-200 g) via Oral, Intravenous and Topical (Cream/Gel) routes; wherein, a comparative qualitative tracking of the blue fluorescence could be recorded. After excipient administration, the major vital organs of the rats; namely, Brain, Lungs, Heart, Kidney, Liver and Spleen were harvested after 3 hrs. and washed with normal saline. The blue fluorescence present in these vital organ tissues was readily observed using a UV Flash Light that confirmed expected excitation and emission at 385 nm and 425 nm, respectively. Although this protocol is qualitative, one could determine distinct organ localizations and relative concentrations of the SupraPlex® Excipients at these sites and one could determine the intensity of the blue fluorescence observed in the respective organs.

When new batches of an excipient were prepared a new Run # was assigned in order to monitor any differences and to accurately track samples during evaluation. Thus, Run #18 appears in repeat batches designated as Runs #182, #183 and #184.

### Example 11: Strategy III: Excipient III: Synthesis, Isolation and Characterization of Blue Fluorescent [CA-CD-tris-Polyol] Poly(ester) Condensates (Run #182)

Reaction of Citric Acid+β-Cyclodextrin+Tris(Hydroxyl Methylaminomethane+NaH₂PO₄.H₂O Using a stoichiometric molar ratio of =[CA:CD:trisPolyol:Catalyst] [6:1:1:1]

Anhydrous citric acid (30.54 g, 0.1585 mole), β-cyclodextrin (30.0 g, 0.0264 moles), TRIS (3.19 g, 0.0263 moles) and sodium dihydrogen phosphate (3.63 g, 02634 moles) were charged into a 500 mL round bottomed flask with 100 mL of DI water to give a homogenous reaction mixture. The physical, unbound water was removed on a Büchi rotavapor under reduced pressure (i.e., 51-120° C./20 mm) over 4-5 hrs. and then held at 130° C./20 mm for 15-20 min. until 5.93 g of chemically bound water formed by esterification (i.e., DE 12.47) produced 61.34 g (91.2%) of a solid cream colored water soluble product. Using a Malvern Nanosizer instrument, a hydrodynamic diameter of 6.56 Nm and Surface Charge of (−12.89 mV) was Observed for Run #182.

### Example 12: Strategy III: Excipients III: Synthesis, Isolation and Characterization of a [CA-CD-Polyol] Poly(ester)Copolymer (Run #65)

Reaction of Citric Acid+β-CD+Glycerin+NaH₂PO4→[CA-CD-Polyol] Poly(Ester) Copolymer Using a Stoichiometric Molar Ratio of [CA:CD:VG Polyol:Catalyst]=[6:1:1:1.3]

Anhydrous citric acid (30.45 g, 0.1585 mole), β-cyclodextrin (30.0 g, 0.02643 mole), glycerin (3.7 g, 0.02643 mole), and sodium dihydrogen phosphate (5.0 g, 0.03628 mole) were charged into a 500 mL round-bottomed flask with 100 mL of DI water to give a homogenous reaction mixture. The physical, unbound water was removed on a Büchi rotavapor under reduced pressure (i.e., 51-130° C./15 mm) over 2-3 hr. and then at 140° C./15 mm for 15-20 min. until 6.93 g of chemically bound water of esterification has been formed to give 62.22 g of sparking white solid product. This crude product was dissolved in 3×50 mL portions of DI water and submitted to ultra-filtration on a 1 kDa membrane to give 35.69 g of sparkling white solid retentate and 26.64 g of cream colored syrupy permeate.

An FTIR analysis of the Run #65 retentate exhibited intense absorption bands at 1733.79 cm⁻¹, 1154.01 cm⁻¹ and 1027.53 cm⁻¹ which are characteristic for ester carbon-oxygen stretching modes.

A ¹³C-NMR analysis of the Run #65 retentate containing products >1 kDa revealed the presence of macromolecular, hyperbranched architecture. ¹³C-NMR carbonyl resonance bands observed at 176.431 ppm, 174.165 ppm, 173.255 ppm and 170.949 ppm supported the presence of ester linkages involved in the formation of these terpolymeric citric acid-β-CD-glycerol compositions which were further characterized and fractionated by Amicon stirred cell ultrafiltration.

Amicon Stirred Cell Ultrafiltration Protocol:

A sample of Run #65 retentate (5.0 g) above was dissolved in 75 mL of DI water. Using an Amicon Stirred Cell Model 8400 Ultrafiltration unit, this solution was filtered using tangential stirred flow under N₂ pressure (˜55 psi) on a 3 kDa membrane (76 mm) until permeation stopped (i.e., ˜10 mL retentate). Water (10 mL) was added and filtration continued until permeation stopped. The permeate was concentrated in vacuo to give 1.0 g of a sparkling white solid. The retentate was washed from the filter with water and concentrated in vacuo on a Büchi rotavapor to give 4.0 g of white solid. This Run #65 retentate (4.0 g) above was dissolved in 75 mL of DI water and filtered on a 5 kDa membrane (76 mm) until permeation stopped (i.e., ˜10 mL retentate). Water (50 mL) was added and filtration continued until permeation stopped. The permeate was concentrated in vacuo by Büchi rotavapor to give 1.2 g of white solid. The retentate was washed from the filter with water and concentrated in vacuo by Büchi rotavapor to give 2.5 g of white solid. Run #65 retentate (2.5 g) above was dissolved in 75 mL water and filtered on a 10 kDa membrane (76 mm) until permeation stopped (i.e ˜5 mL retentate). Water (50 mL) was added and filtration continued until permeation stopped. The permeate was concentrated in vacuo via Büchi rotavapor to give 1.8 g of white solid. The retentate was washed from the filter with water and concentrated in vacuo with a Büchi rotavapor to give 0.8 g of sparkling white solid.

In summary, Amicon membrane fractionation of a 5.0 g sample of Run #65 retentate using specific MWt cut-off membranes produced the molecular weight distribution results with a material balance of 96% as described in Table 13.

TABLE 13

| kDa (MWt) | Grams |
|---|---|
| 1-3 | 1.0 |
| 3-5 | 1.2 |
| 5-10 | 1.8 |
| >10 | 0.8 |

Evaluation Polymer Adduct Solubility Enhancement Performance

A typical β-CD based SupraPlex® Excipient such as Run #65 (i.e., retentate), revealed invaluable solubility enhancement properties for important APIs as shown in FIG. 7. Discrete solubility enhancement properties unique to the combination of the API guest structure, as well as specific excipient compositions. For example, Run #65 (i.e. citric acid-β-CD-glycerin) Excipient composition was evaluated against 21-different insoluble active pharmaceutical ingredients (APIs). These guest API's included: anti-oxidants, flavonoids, cannabinoids, non-steroidal anti-inflammatory agents, steroids, nutrient/vitamins and natural flavors and appeared to yield solubility enhancement properties that were dependent on the guest size, shape and surface chemistry interactions with a specific excipient composition as shown in FIG. 7.

Examination of at least 10 different Polymeric Adducts having Excipient (I)-(IV) type, and CA-CD-Polyol revealed similar discrete and important structure-solubility enhancement activity relationships. Evidence for this hypothesis was gained by comparing specifically engineered Excipient compositions such as Runs #59, #60-#62, #66, #67, and #118-#121 against this same repertoire of 21 APIs used for Run #65 (shown in FIG. 7). These results are as illustrated in FIGS. 8-17.

These solubility enhancement data were found to be directed inextricably by certain critical excipient compositions defined by unique reaction stoichiometries and other reaction parameters. These parameters included: the size of the parent α-, β- and γ-cyclodextrin cavities, type of poly(hydroxylic) alcohol monomer used, their stoichiometry ratios relative to citric acid, as well as the specific reaction protocols and conditions used (i.e., reaction temperatures/times, catalyst type/stoichiometries, etc.) (see FIGS. 5-6). As such, it soon became apparent that these critical parameters could be systematically engineered to define optimized Excipient compositions for any desired or targeted APIs.

To attain the desired increased API solubilities a copolymeric host structures (PHC) may be designed and engineered that comprise a linear, random branched, hyperbranched or dendritic oligomer/polymer architecture; wherein, the co-monomers are glucose based poly(hydroxylic) alcohols (i.e., α, β and γ-cyclodextrins/optional low MWt<500 Da poly(hydroxylic) alcohols (i.e. glycerin, sorbitol, TRIS, pentaerytheritol, etc.) and poly(carboxylic) acids (i.e., citric acid, tartaric acid, etc.). These sugar-based poly(hydroxylic) alcohols may be any water soluble, functionalized poly(hydroxylic) alcohol comprising α, β, or γ-cyclodextrins; wherein, the cyclodextrin component may allow at least two appended carboxylate groups derived from carboxylic acids, esters, or activated esters and may include various α-, β-, γ-cyclodextrins, 2-[hydroxypropyl] β-cyclodextrin (2-HP-CD), random methylated β-cyclodextrin (MO-CD), sulfonated β-cyclodextrin or other functionalized cyclodextrins.

Comparing the CBD solubility enhancements of the top 25 SupraPlex® Excipients (Table 1) against the reported literature value for naked CBD solubility in DI water (i.e., 0.0627m/mL) (Koch, N. et al., Inter. J. Pharm., 2020, 589, 119812) reveals that solubility enhancements range from 70,175× fold (Run #93) to 240,829× fold (Run #108) in this excipient series.

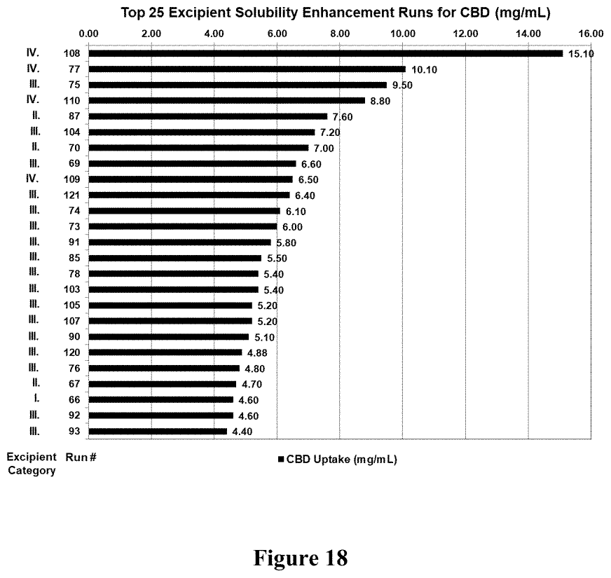

Several important targeted active ingredients such as CBD, curcumin and resveratrol were extensively evaluated. More specifically, the solubility enhancement of CBD was systematically evaluated by combining to form >100 different Polymeric Adducts involving SupraPlex® Excipient (I)-(IV) types with various CA-CD-Polyol compositions. This examination defined the top 25 most active SupraPlex® Excipient compositions and produced CBD solubility enhancements ranging from 4.4 mg/mL-15.1 mg/mL as illustrated in FIG. 18.

Essentially all of the top candidates were random methylated β-CD (Meβ-CD) based compositions and these active copolymeric compositions were obtained using all three inorganic phosphate catalyst systems (i.e., Na₂HPO₄, NaH₂PO₄ or NaH₂PO₂) and involving [CA:CD:polyol:catalyst] molar stoichiometries ranging from [3:1:1:1] to [7:1:1:4.3], respectively. Sixteen of the top 25 SupraPlex® Excipients were Type III; CA-Meβ-CD-Polyol compositions containing glycerin, d-sorbitol or pentaerythritrol comonomers with degrees of esterification ranging from 1.47-28. The top candidate (i.e., Run #108; 15.1 mg/mL), as well as three other Excipients residing in the top nine candidates; namely: Run #77 (10.1 mg/mL); Run #110 (8.8 mg/mL) and Run #109 (6.5 mg/ml), were Type IV; SupraPlex® Excipients that were obtained by final surface modification of Type (I)-(III) Excipients with glycerin (see FIGS. 5 and 6).

Comparing solubility enhancements of these top 25 SupraPlex® Excipients against the literature value for the solubility of CBD in DI water (i.e., 0.0627 μg/mL) (Koch, N. et al., Inter. J. Pharm., 2020, 589, 119812) reveals that the SupraPlex® assisted solubility enhancements ranged from 70,175×fold (Run #93) to 240,829 fold (Run #108) for this Excipient series.

Using eleven different arbitrarily selected SupraPlex® Excipient compositions (i.e. Runs #59-121) as described in Table 14, were combined with 3 targeted active ingredients; namely: CBD, resveratrol or curcumin. They were then examined to determine specific water solubility enhancement properties as a function of excipient composition and reaction parameters used for their syntheses.

TABLE 14
SupraPlex ® Excipient

| Run # | Cyclodextrin/Polyols | Catalyst |
|---|---|---|
| 59 | 2-[HPCD]+ | NaH₂PO₄ |
| 60 | [α-CD] + 1.5 hr. (89-135° C./18 mm); DE = 8.46 | NaH₂PO₄ |
| 61 | [α-CD] + 2.0 hr(82-133° C./17 mm); DE = 9.17 | NaH₂PO₄ |
| 62 | [α-CD] + 2.5 hrs(85-134° C./17 mm): DE = 9.30 | NaH₂PO₄ |
| 65 | [β-CD] + glycerin+ | NaH₂PO₄ |
| 66 | [Meβ-CD]+ | NaH₂PO₄ |
| 67 | [Meβ-CD]+ | NaH₂PO₄ |
| 118 | [Meβ-CD] + glycerin+ | Na₂HPO₄ |
| 119 | [Meβ-CD] + glycerin+ | Na₂HPO₄ |
| 120 | [Meβ-CD] + glycerin+ | NaH₂PO₂ |
| 121 | [Meβ-CD] + glycerin+ | Na₂HPO₄ |

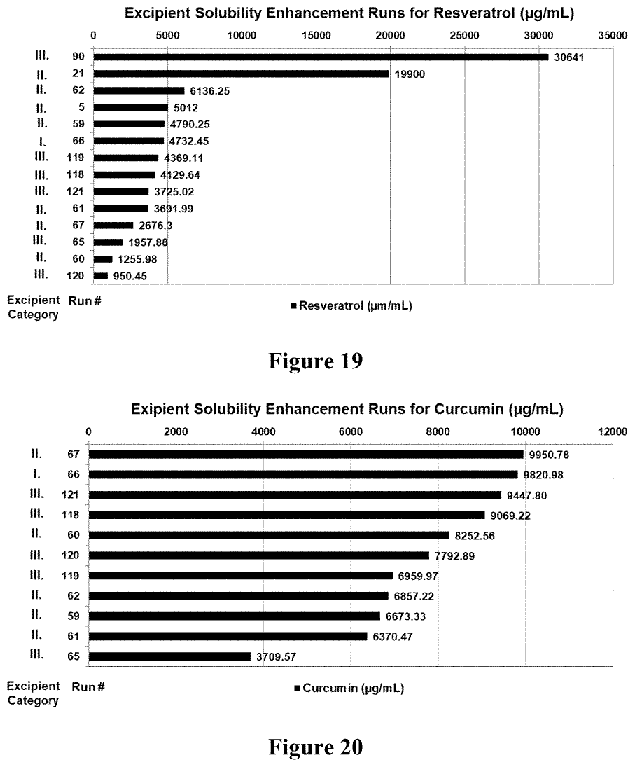

Comparing solubility enhancements observed for the top SupraPlex® Excipients in this series (FIG. 19) against the reported literature value for the naked resveratrol solubility in DI water (i.e., 0.04 μg/mL) (A. Chauhan, et al., US. Patent #2016/0206572 A1, Jul. 21, 2016) reveals that the excipient assisted solubility enhancements ranged from 23,761×-fold (Run #120) to 766,025×-fold (Run #90) within this Excipient series.

Comparing solubility enhancements observed for the top SupraPlex® Excipients in this series (FIG. 20) against the reported literature value for naked curcumin solubility in DI water (i.e., 2.67 μg/mL) (Modasiya, M. K. et al., Int. J. Pharm. & Life Sci., 2012, 3(3), 1490-1497) reveals that excipient assisted solubility enhancements ranged from 1389×-fold (Run #65) to 3727×-fold (Run #67) within this Excipient series. The top 4 candidates involved poly(ester) copolymer compositions containing MeβCD and glycerin (Runs #121, 118), as well as MeβCD without glycerin (Runs #67, 66). Three candidates containing α-CD components produced excipient assisted (Runs #60, 61, 62) enhancements that ranged from 2386-3091×-fold. Other SupraPlex® Excipients in this series containing 2-[HPβ-CD] (Run #59) or β-CD (Run #65), respectively enhanced curcumin solubility by 2499×-fold and 1389×-fold, respectively.

It is both remarkable and important to note that one can readily obtain quantitative structure activity relationships (QSARs) for these SupraPlex® excipient structures by simply comparing the excipient assisted solubility enhancement values for specific excipient composition within any defined SupraPlex® excipient series. This provides a very powerful tool/strategy for optimizing and expanding SupraPlex® Excipient applications for a wide variety of specific hydrophobic guest properties used in current as well as new products. These QSAR type evaluations provide a valuable feed-back loop for systematically engineering SupraPlex® Excipients to fulfill unmet needs for active ingredients solubility enhancements, protection against photo/chemical degradation, extended shelf stabilization, bioavailability, controlled release features, optimum dosage levels as well as modes of administration.

### Example 13: Excipient Assisted Resveratrol Solubility Enhancement

An arbitrary collection of fourteen different SupraPlex® Excipient compositions were examined as polymeric host compounds (PHCs) for enhancing the water solubility of resveratrol. A forced ranking of these 14 Excipients as a function of solubility enhancement values is as illustrated in FIG. 19. This ranking revealed that 7 of 14 Excipient candidates were Type II excipients (i.e., Runs #21, 62, 5, 59, 61, 67 and 60) and 6 of the 14 candidates were Type III Excipients (i.e., Runs #90, 119, 118, 121, 65 and 120) and dominated the resveratrol solubility enhancement activity, with only one example of a Type I (i.e., Run #66) and no examples of Type IV Excipients being represented in this series. Among the top five most active candidates, 4 of 5 were Type II Excipients derived from citric acid-α, β- or 2-hydroxypropyl-β-CDs, copolymers, respectively, with only one example of a Meβ-CD+glycerin Type III candidate. This specific Type III example was the top candidate in this series giving a resveratrol solubility enhancement value of 30641 μg/mL (i.e., —750,000×-fold solubility enhancement).

### Example 14: Excipient Assisted Curcumin Solubility Enhancement

An arbitrary list of eleven SupraPlex® Excipients were examined as polymeric host compounds (PHCs) for enhancing the water solubility of curcumin. A forced ranking of these 11 Excipients as a function of solubility enhancement is as illustrated in FIG. 20. This ranking revealed that 5 of 11 Excipient Types (II) (i.e., Runs #67, #60, #62, #59 and #61) and 5 of 11 Excipient Type III (i.e., Runs #121, #118, #120, #119, and #65) dominated this solubility enhancement activity with only one example of a type I (i.e., Run #66) and no examples of Type IV being represented in this list. Among the top 5 most active candidates, 2 of 5 (i.e., Runs #67 and #60) were Type II and 2 of 5 (i.e., Runs #121 and #118) were Type III with only one example of a Type I Excipient (i.e., Run #66). It is interesting to note that 4 of 5 of the top candidates are based on simple Meβ-CD copolymers (i.e., Runs #67, #66, #121 and #118) and one 1 of 5 is based on a simple citric acid-α-CD copolymer.

### Example 15: Protocols for Accelerated Storage Stability Studies Protocol

Short term accelerated stability testing of Polymeric Adducts containing either Resveratrol (RSV) or Cannabidiol (CBD) was performed both in liquid and lyophilized state at RT and 50±0.5° C. using a VWR Model 1300U oven over a period of 30 days. Samples were stored in clear colorless glass vials at RT, as well as at elevated temperature for specific time periods and were visually monitored for any propensity to undergo precipitation. crystallization, develop turbidity or exhibit change in consistency. Active ingredient guest concentrations were measured by UV-Vis spectrometry (U-3010, Hitachi, Japan).

The storage stability of the Polymeric Adduct was examined in the dark at RT at 50±0.5° C. for a 30-day period. Any change in the active ingredient content was regularly monitored on the 7^{th}, 15^{th} and 30^{th} day. It was observed that the active ingredient concentration of the liquid and lyophilized samples remained relatively constant at RT, thus indicating that Run #90 RSV in the liquid state at 50±0.5° C. and Run #108 CBD in the lyophilized state at RT have exhibited high stability which is very favorable for a wide range of in vitro, ex-vivo and in vivo applications.

Furthermore, these aqueous solubilized pharmaceutical products were visually monitored for any physical evidence of instability. Certain nominal signs such as turbidity, precipitation, or change in consistency were observed after 30 days in all the RSV and CBD formulations. The degradation rate constant was very low with lyophilized Run #90 RSV and Run #108 CBD, showing the chemical stability at 50±0.5° C. (Table 15).

TABLE 15

| | Parameters | | | | | | | |
|---|---|---|---|---|---|---|---|---|
| | % Drug Content | | % Drug Loss | | Precipitation | | Turbidity | |
| Sample Code | Day 1 | Day 30 | Day 1 | Day 30 | Day 1 | Day 30 | Day 1 | Day 30 |
| #90 RSV | 100 | 96.62 | 0 | 3.38 | NC | CC | NC | CC |
| (Liquid @ RT) | | | | | | | | |
| #90 RSV | 100 | 85.97 | 0 | 14.03 | NC | CC | NC | CC |
| (Liquid @ 50 ± 0.5° C.) | | | | | | | | |
| #90 RSV | 100 | 92.74 | 0 | 7.26 | NC | NC | NC | NC |
| (Lyophilized @ RT) | | | | | | | | |
| #90 RSV | 100 | 86.55 | 0 | 13.45 | NC | NC | NC | NC |
| (Lyophilized @ 50 ± 0.5° C.) | | | | | | | | |
| #108 CBD | 100 | 88.96 | 0 | 11.04 | NC | CC | NC | CC |
| (Liquid @ RT) | | | | | | | | |
| #108 CBD | 100 | 89.79 | 0 | 10.21 | NC | CC | NC | CC |
| (Liquid @ 50 ± 0.5° C.) | | | | | | | | |
| #108 CBD | 100 | 96.96 | 0 | 3.04 | NC | NC | NC | NC |
| (Lyophilized @ RT) | | | | | | | | |
| #108 CBD | 100 | 90.83 | 0 | 9.17 | NC | NC | NC | NC |
| (Lyophilized @ 50 ± 0.5° C.) | | | | | | | | |

NC = no change,
CC = considerable change

### Example 16: Using Combinations of Two or More Active Ingredient Guest Molecules in Various PHCs

The potential for using SupraPlex® excipients in combination therapies for the delivery of two or more active ingredients using two approaches:

1. Simultaneous encapsulation technique

2. Simple mixing technique

The simultaneous encapsulation technique involves the simultaneous addition of more than one Guest molecule to one or more polymeric host compound(s). The mixing technique involves a two-step process; wherein, a polymer adduct solution is prepared for each individual Guest molecule followed by combining and further mixing of these solutions as described below:

### Example

1. RSV, CBD and CUR were physically entrapped with SupraPlex® excipient (i.e. Run #65) as previously described to form a mixed Polymeric Adduct.
2. Run #65 RSV, Run #65 CBD and Run #65 CUR Excipient Complexes were prepared as previously described. The solutions were combined in a 1:1:1 ratio and further mixed by shaking to form a mixed Polymeric Adduct.
3. Analysis of the mixed Polymeric Adducts derived from two methods showed identical properties.

### Example 17: Excipient Type IV Experimental Protocol

Category Type IV Excipients are readily synthesized by post reaction of carboxylate terminated Category Type I, II or III Excipients with an excess of a suitable polyol (i.e., glycerin, d-sorbitol, propylene glycol, glucose, pentaerythritol or cyclodextrin bearing primary hydroxyl functionality). Experimental examples in Table I included: Runs #77, #108, #109 and #110.

A typical example of a Type IV Excipient synthesis is as follows:

Citric acid (10.13 g, 0.0528 mol), random methylated β-CD (17.22 g, 0.0132 mol) and disodium hydrogen phosphate (2.5 g, 0.01818 mol) were charged into a 500 mL round-bottomed flask with 50 mL of DI water. A homogeneous solution was obtained by stirring with slight heating. Physical, unbound water was removed on a Büchi rotavapor at 25-95° C./20 mm over a period of 1 hr., followed by heating from 95° C. to 142° C./14 mm over 1 hr. and then holding at 142° C./14 mm for 15 min. to give a water soluble, white crude product with no insoluble cross-linked side products. Wt.=29.16 g (i.e. a weight loss of 0.69 g compared to charged reactants; degree of esterification (DE)=2.90). Both FTIR and ¹³C-NMR confirm a surface carboxylated CA-CD copolymeric product. This crude product (28.34 g) was combined with 10.2 g of glycerin in 20 mL of DI water and heated at 120° C. for 30 min. and then stripped free of water at 100-121° C./100 mm over a period of 1 hr. to give a clear transparent, syrup, Wt.=39.02 g. This syrup was then diluted with about 100 mL of DI water and fractionated on a UF filtration device (i.e., using a 1 kDa membrane) to give 13.27 g of retentate (i.e., a white sparkling solid) and 22.67 g of permeate (i.e., a cream colored syrup) that appeared to contain a substantial amount of unreacted glycerin. This Type IV Excipient product was examined by FTIR and ¹³C-NMR which confirmed the loss of carboxylate moiety accompanied by an increase in symmetrical hydroxyl functionality (i.e., carbonyl ester at 173.194 ppm). Evaluation of this product as an excipient for the solubility enhancement of CBD involves the use of our standard UV based solubility enhancement protocol. This analysis, revealed solubility value for CBD uptake of 8.8 mg/mL.

### Example 18: Patterns/Trends for Active Versus Inactive SupraPlex® Compositions as CBD Water Solubility Enhancers

A combinatorial library containing over 194 examples of SupraPlex® based

Excipient and Polymeric Adduct compositions were synthesized according general protocols described above; wherein, three critical reaction parameters were carefully monitored and described below. Stoichiometric ratios for reactants defined as:
1. [Citric acid:CD1:CD2:polyol:catalyst] reaction
2. Optimized reaction temperatures/reduced pressures/reaction time cycles
3. Degree of esterification (DE)

These parameters were varied systematically, according to the Run # (i.e., Runs #1-#194) to produce a wide range of discrete SupraPlex® compositions which, in each case, corresponded to a specific Excipient Category Types I-IV. As such, these respective Runs #1-194 were evaluated quantitatively to yield solubility enhancement values for CBD uptake in DI water using a standardized UV assessment protocol as described earlier in this specification. These quantitative solubility enhancement values for CBD were then force ranked to select the top 25 most active candidates out of Runs #1-194, using a QSAR-type (i.e., quantitative structure-activity relationship) evaluation format as shown in FIG. 18.

It is noteworthy, that all four of the Excipient Type IV candidates (i.e., Runs #108, #77, #110, #109) in FIG. 18 are among the most active solubility enhancement candidates. This is followed by three Excipient Type II candidates (i.e., Runs #87, #70, #67), that is next followed by 17 Excipient Type III candidates (i.e., Runs #75, #104, #69, #121, #74, #73, #91, #85, #78, #103, #105, #107, #90, #120, #76, #92, #93) with only one Excipient Type I (i.e., Run #66) appearing third from the bottom of this list. This activity pattern suggests that glycerin terminated Excipients (i.e., Type IV) are more preferred than carboxylate terminated (i.e., Types I, II, III) for optimum activity. Furthermore, it should be noted that a majority of the most active Excipient candidates are derived from random methylated CDs or random methylated CD terpolymers involving citric acid and glycerin. The least represented and lower activity Excipient in this top 25 list was a Type I Excipient (i.e., Run #66). Preferred citric acid stoichiometries relative to the CDs=component that produced the most active candidates ranging from 3-7. In contrast, examination of a forced ranking of all examples in Runs #1-194 showed that using high molar excesses of citric acid relative to CD (i.e., 12-24 molar excess) produced Excipient candidates with some of the lowest solubility enhancement values. As such, it might be expected that using these large CA excesses may have led to very high CD surface esterification that not only precluded formation of CA-CD copolymerization, but also sterically hindered access of CBD guest molecules into the CD cavities for optimum encapsulation/confinement.

### Example 19: Stability Studies for CBD Based Polymeric Adducts in Contact with Plastic Substrates

### UV Spectrometry Protocol for Monitoring CBD Loaded SupraPlex® Excipients in the Presence of Plastic Substrates

The use of UV spectroscopy for monitoring CBD loaded SupraPlex® in aqueous solutions is very suitable for quantitative determination of CBD concentration levels in solution. The λₘₐₓ of CBD was determined by using approximately 500 μg/mL of CBD in methanol and recording the spectrum in the spectral range of 200-400 nm using a double beam UV spectrophotometer (Hitachi U-3010, Japan). The UV spectrum of CBD is shown to exhibit a λₘₐₓ at 273-4 nm. A standard working solution of CBD was prepared in methanol and appropriately diluted to create a calibration curve to cover concentration from the 50 μg/mL to 500 μg/mL with a regression square value of at least 0.9999.

The test solution of CBD (15 mg) in SupraPlex® (i.e., 7 gm in 300 mL) was loaded into a PETE test bottle and the void space flushed with nitrogen. The bottle was then agitated on an orbital platform shaker. Aliquots were taken from the bottle on 0, 1, 2, 7, 15 and 30 day intervals and the UV-Vis spectra were measured against a water reference. Spectra showed the characteristic double peak for CBD and absorbance at 273-4 was essentially unchanged over a seven day time period. Changes in absorptivity and λₘₐₓ between the two solvent systems (methanol vs. aqueous SupraPlex®) appear to be minor.

Prolonged 30-day, plastic substrate stability studies were performed at ambient temperature using Run #168 (i.e., 2% w/w SupraPlex®) loaded with 12.5 mg CBD in 300 mL of diluent. Run #168 is a kg scale-up using procedures developed earlier for Runs #65 and #147. The loaded SupraPlex® samples were examined in both plastic water bottles (PETE) and plastic coated beverage cans by dissolving directly into DI water (Table 16), as well as into various beverages such as distilled water (DI) or Diet 7UP (Table 16). The loaded samples were stored at RT.

One set of samples was allowed to shake on an orbital shaker (i.e., RotoMix Model 48200, Thermolyne, at 120 rpm) and the other set was kept in a stationary mode, in each case at room temperature. Samples were stored for the whole duration of the 30 days' experiment at RT and were regularly monitored through the 30^{th} day and beyond. They were monitored for various physical stability parameters such as precipitation, crystallization, development of turbidity, coating to the plastic substrate or any change in consistency. Aliquots of the samples were withdrawn at various time intervals and the concentration level of CBD was measured by a UV-Vis spectrometry (U-3010, Hitachi, Japan) at 273-274 nm. Although some fluctuation in the CBD signal (i.e., 273-274 nm) was noted during the first 7 days of the study, this signal leveled off and remained steady throughout the remainder of the 30-day monitoring.

TABLE 16
Run #168 + CBD

| | | DI water in | |
|---|---|---|---|
| | DI water in PETE | Plastic Lined | Diet 7UP in PETE |
| | Plastic Bottle | Beverage Can | Plastic Bottle |
| | pH = 2.5 ± 0.5 | pH = 2.5 ± 0.5 | pH = 3.5 ± 0.5 |
| Days | % CBD Content | % CBD Content | % CBD Content |
| 0 | 100 | 100 | 100 |
| 1 | 117.74 | 78.34 | 96.9 |
| 2 | 95.67 | 52.22 | 108.46 |
| 7 | 118.61 | 112.73 | 132.30 |
| 15 | 106.49 | 94.26 | 96.15 |
| 30 | 104.76 | 94.90 | 97.69 |
| 365 | 89.61 | 84.07 | 94.61 |

It was observed that 2% w/w SupraPlex®, Run #168, loaded with 12.5 mg of CBD remained clear and transparent. There were no physical indications of heterogeneity due to precipitation or filming out on the plastic substrate. According to UV analysis, essentially no UV signal loss for CBD (i.e., ±10%) (Table 16) was observed over the one month study involving contact with plastic coated beverage cans or with plastic water bottles either with continuous stirring or in the stationary mode at room temperature.

It was observed that 2 wt % SupraPlex® Excipient, Run #168, dissolved in various aqueous media (i.e., DI water or Diet 7UP®) could be readily loaded with 12.5 mg of CBD in 300 mL of liquid at room temperature either at pH 2.5±0.5 or 3.5±0.5. These formulations were then exposed to various plastic surfaces over a period of 365 days (12 months) as shown in Table 16. They remained clear/transparent and deviated no more than ±10% from the original CBD loading level as described below:
Run #168 with 12.5 mg CBD in 300 mL of DI water in a PETE plastic bottle at pH 2.5±0.5. Table 14 showed a deviation of about 5.4% from original concentration after 1-year at RT.
Run #168 with 12.5 mg CBD in 300 mL of DI water in a plastic lined beverage can at pH 2.5±0.5. Table 14 showed a deviation of about 15.03% from original concentration after 1-year at RT.
Run #168 with 12.5 mg CBD in 300 mL of Diet 7UP contained in a sealed PETE plastic bottle at pH 3.5±0.5. Table 14 showed a deviation of about 2.3% from original concentration after 1-year at RT. Table 14.

No physical evidence or spectroscopic indication that 2% w/w SupraPlex®; Run #168, loaded with 12.5 mg CBD (300 mL) exhibits any heterogeneity throughout the 30-365 day stability study. Both UV and Terahertz spectroscopy have provided corroborating spectroscopic evidence that essentially no CBD depletion other than experimental error occurs under these conditions (i.e., ±10%) or that any instability is occurring. These are data confirming a shelf life stability of at least 30-365 days (i.e., 1-year) for CBD loaded SupraPlex®; Run^{#}168 in direct contact with various plastic substrates. Similarly, when the CBD (12.5 mg) was loaded into 300 mL of 2 wt % SupraPlex® Excipient (i.e. Run #168 in DI water) no solution depletion or loss of stability was observed when stored in a sealed PETE plastic bottle at RT for 30-365 days (1-year).

Also, a sample of CBD (1.117 mg/mL; pH 3.5±0.5 was loaded into 20 wt % of SupraPlex® excipient concentration (i.e. Run #172 in DI water). This formulation exhibited no solution depletion or loss of stability when stored in a sealed poly(propylene) plastic vial at RT for 9 months (Table 17).

TABLE 17

| | Run #172-CBD |
|---|---|
| | DI water in PETE Plastic Vial |
| | pH 3.5 ± 0.5 |
| Months | % CBD Content |
| 0 | 100 |
| 1 | 91.33 |
| 3 | 86.45 |
| 6 | 95.66 |
| 9 | 93.49 |

### Example 20: Formulations of Resveratrol or Curcumin in 20Wt % SupraPlex® Excipient Formulations

Samples of cannabidiol, resveratrol and curcumin, respectively loaded in 20 wt % SupraPlex® excipient concentrations (i.e. Run #65 in DI water; pH 2.5±0.5). These formulations exhibited essentially no solution depletion when stored in a sealed poly(propylene) plastic vial at RT for 12 months. However, after 18 months about 18-21% depletion from original concentration was observed according to UV spectrometry (Table 18).

TABLE 18

| | #65 Cannabidiol | #65 Resveratrol | #65 Curcumin |
|---|---|---|---|
| | pH 2.0 ± 0.5 | pH 2.0 ± 0.5 | pH 2.0 ± 0.5 |
| Months | % CBD Content | % RSV Content | % CUR Content |
| 0 | 100 | 100 | 100 |
| 3 | 93.28 | 97.46 | 96.20 |
| 6 | 88.49 | 84.39 | 79.58 |
| 9 | 96.16 | 89.45 | 96.79 |
| 12 | 87.05 | 93.67 | 92.56 |
| 18 | 94.72 | 82.70 | 79.88 |

Samples of cannabidiol, resveratrol and curcumin, respectively, were loaded into 20 wt % SupraPlex® excipient concentrations (i.e. Run #65 in DI water; pH 2.5±0.5). These formulations exhibited essentially no solution depletion when stored in a sealed poly(propylene) plastic vial at RT for 12 months. However, after 18 months about 18-21% depletion from original concentration was observed according to UV spectrometry (Table 15).

### Example 21: Method of Use for SupraPlex® Based Polymer Adducts

The PHC's made above may be used to incorporate essentially any active ingredient type Guest molecules possessing appropriate sizes, shapes and hydrophobic features that match the CD cavity space in the PHC's. This provides a versatile delivery platform for hydrophobic or water insoluble Guest molecules by making them more water soluble and bioavailable to the cells' receptor sites in humans and animals (e.g., in vivo, in vitro, or ex vivo). There are many examples of such important hydrophobic Guest molecules that include CBD, THC, or other Hemp compounds as well as many natural products. The use of PHC's to produce polymeric adducts containing hydrophobic Guest molecules provides a wide range of unique non-toxic water-soluble delivery systems; wherein, the PHC may be expected to be a GRAS certified composition when derived from FDA approved CD, CA and glycerin components. Essentially any Guest molecules that can spatially fit the interior void spaces of the PHC may lead to water soluble Polymeric Adducts suitable for the delivery and release of important active hydrophobic ingredients critical for medical therapies, personal care/cosmetic products and vitamins/nutrients to mention a few.

Thus, typical and useful Polymeric Adducts derived from PHC-type Excipients and active Guest molecules are largely dependent upon the proper selection of critical design parameters that include suitable molecular level sizes, shapes and surface chemistries. Encapsulation of a wide range of Guest molecules in PHC's to form corresponding Polymeric Adducts is possible, but is largely dependent on the proper matching of key design parameters (i.e., sizes, shapes and surface chemistries) associated with the Guest molecules and interior void space available in the PHC. As such, critical design parameters of PHC-type Excipients may be varied as a function of the CD cavity size, as well as unique interior void space defined by the architecture of the PHC as illustrated in FIG. 3 as well as by the architecture of the Excipient Type (i.e. (I), (II), (III) and IV) as defined in FIG. 5. These SupraPlex® type PHC's may be precisely engineered to fulfill many unmet needs associated with product development and commercial applications in the life sciences, agriculture, diagnostics, pharmaceuticals, food-beverage industry, pet food, veterinary, dentistry, nutraceuticals, cosmetics, cosmeceuticals, personal care, aromatherapy and fragrance industries.

### Example 22: In Vitro Dissolution of Polymer Adduct Protocols

The in vitro dissolution of Polymer Adducts (i.e. Run #90 RSV and Run #108 CBD) were performed in 0.1 N of HCl at pH 1.2. Simulated Gastric Fluid (SGF pH 1.2), Phosphate Buffer (PB pH 6.8) and Simulated Intestinal Fluid (SIF pH 6.8) as dissolution medium. Briefly, lyophilized Polymer Adducts (i.e. Run #90 RSV and Run #108 CBD) solids containing Guest molecule levels equivalent to 1 mg of naked RSV or CBD was added into 100 mL of dissolution media and stirred on at a fixed rate at 37±0.5° C. At predetermined time intervals, 1 mL samples were withdrawn and replenished with the same volume of fresh medium. The RSV and CBD content in these samples were determined by using a UV-visible Spectrophotometer (U-3010, Hitachi, Japan) and specific amounts of RSV and CBD dissolved and were monitored and estimated as a function of time.

Both Run #90 RSV and Run #108 CBD exhibited enhanced in vitro dissolution performance compared to the naked RSV and CBD which led to saturation and incomplete dissolution profiles in all media that were examined.

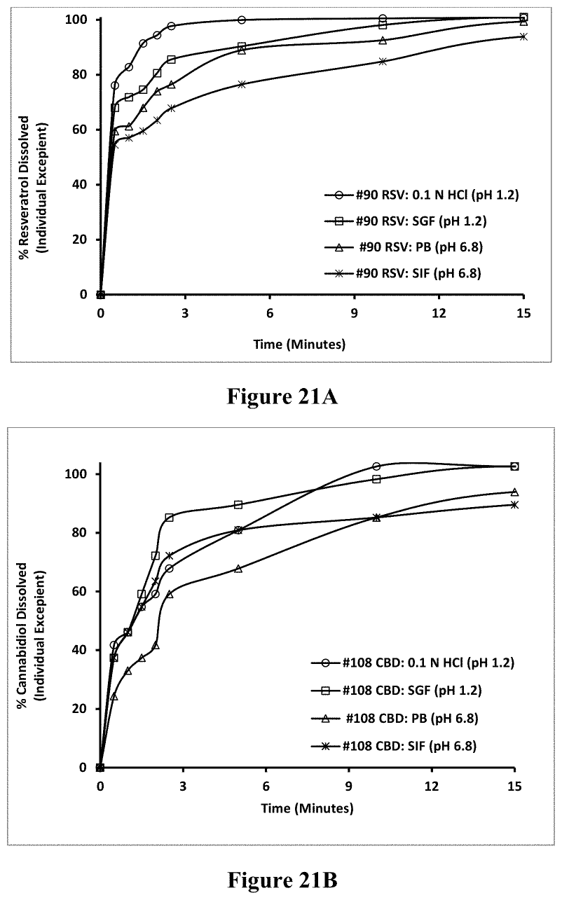

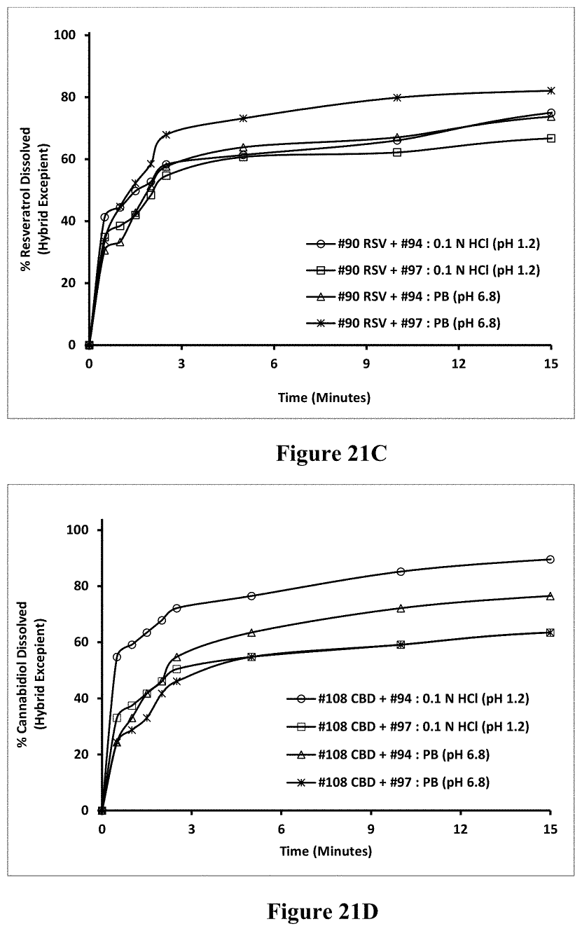

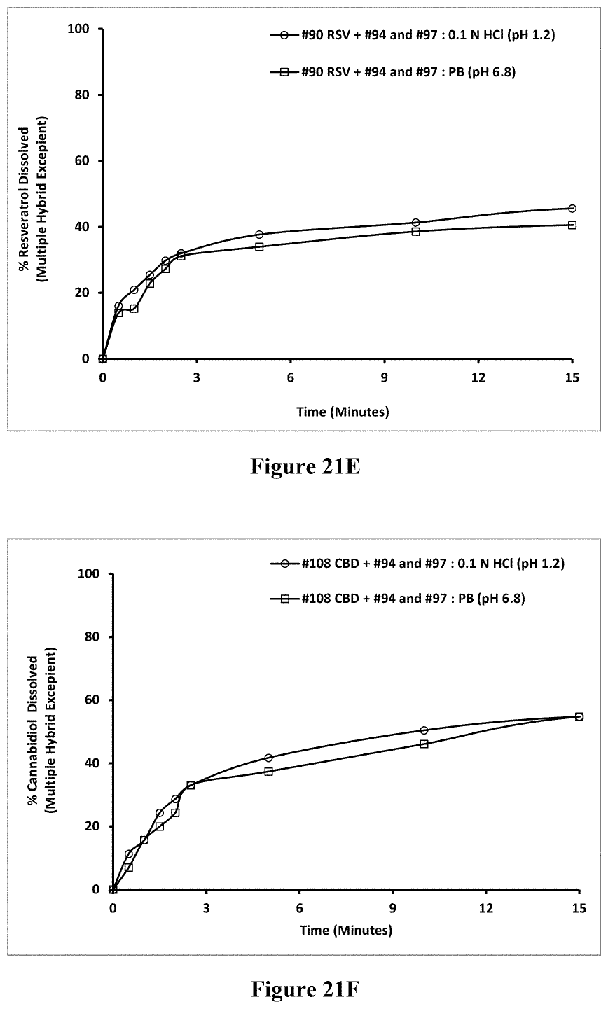

Polymeric Adduct (i.e. Run #90 RSV) dissolved more readily than insoluble naked RSV in 0.1N HCl (pH 1.2), SGF (pH 1.2), PB (pH 6.8) or SIF (pH 6.8). Using 0.1N of HCl (pH 1.2) or SGF (pH 1.2) dissolution of Run #90 RSV occurred rapidly compared to PB (pH 6.8) and SIF (pH 6.8). For example, it was observed that 100% of Polymer Adduct (i.e., Run #90 RSV dissolved in 0.1N of HCl (pH 1.2) or SGF (pH 1.2) within 10 min. The dissolution of Polymer Adduct (i.e. Run #90 RSV) was 92.57% and 84.81%, respectively in PB (pH 6.8) or SIF (pH 6.8) and continued to progress toward 100% dissolution after 15 min. (FIG. 21A).

Whereas, naked CBD was insoluble, a facile dissolution of Polymer Adduct (i.e., Run #108 CBD) occurred in 0.1N of HCl (pH 1.2), SGF (pH 1.2), PB (pH 6.8) or SIF (pH 6.8) and exhibited a similar dissolution profiles compared to Polymer Adduct (i.e., Run #90 RSV). It was observed that 100% of Polymer Adduct (i.e. Run #108 CBD) dissolved in 0.1N of HCl (pH 1.2) or SGF (pH 1.2) within 15 min. The dissolution of Polymer Adduct (i.e., Run #108 CBD) was 93.91% and 89.56% in PB (pH 6.8) and SIF (pH 6.8), respectively and still progress toward 100% dissolution after 30 min. (FIG. 21B).

### Example 23: In Vitro Dissolution Protocol for SupraPlex® Based Hybrid Excipients

The Polymer Adducts (i.e., Run #90 RSV and Run #108 CBD) were mixed with individual insoluble, crosslinked Run #94 and Run #97, at a volume ratio of 1:1 and stirred overnight at RT to form a Hybrid Excipient. In order to form a more complex, multiple Hybrid Excipients, a 1:1 blend of Polymer Adducts (i.e., Run #90 RSV and Run #108 CBD) was mixed with insoluble, crosslinked nanosponge compositions (i.e. Run #94 and Run #97) and stirred overnight at RT. This multiple, hybrid excipient was then lyophilized and used for dissolution studies.

Combination of Polymer Adducts (i.e., Run #90 RSV and Run #108 CBD) with insoluble, crosslinked nanosponge compositions (i.e. Run #94 or Run #97) produced the corresponding Hybrid Excipients. Dissolution was performed in 0.1N of HCl (pH 1.2) or PB (pH 6.8). Dissolutions in either media were slower compared to non-hybrid examples. For example, 74.98% dissolution occurred for Run #90 RSV+Run #94 and 66.72% dissolution occurred for Run #90 RSV+Run #97 at pH 1.2 followed by 73.74% dissolution for Run #90 RSV+Run #94 and 82.08% dissolution occurred for Run #90 RSV+Run #97 at pH 6.8 after 15 min. (FIG. 21C). Similarly, dissolution of 89.56% was achieved for Run #108 CBD+Run #94, 63.47% for Run #108 CBD+Run #97 in pH 1.2 followed by 76.52% dissolution for Run #108 CBD+Run #94 and 63.47% dissolution was observed for Run #108 CBD+Run #97 at pH 6.8 (FIG. 21D) over the same 15 min. time period.

The combination of insoluble Run #94 and Run #97 (multiple Excipient), with Run #90 RSV displayed the slow dissolution of 45.59% and 40.55% in the pH 1.2 and pH 6.8 buffers for 15 min. and 54.78% dissolution for Run #108 CBD in both (pH 1.2 and pH 6.8) buffers (FIGS. 21E and 21F).

### Example 24: In Vitro Drug Release Protocol

Dissolution profiles help to understand the pattern of drug-complexes dissolving in the dissolution medium, whereas in vitro release studies give the profile of drug release from dissolved drug-complexes.

Thus, in order to mimic the biological system and pH the in vitro release by dialysis tubing method (MWCO-1 kDa, Spectra/Por Dialysis Membrane, USA) was used for determination of the release profile for various Run #90 RSV and Run #108 CBD combinations in Phosphate Buffered Saline (PBS pH 7.4). Briefly, 1 mg of Run #90 RSV and Run #108 CBD was introduced into a dialysis bag in 100 mL of release media and stirred on a stir plate at constant rate at 37±0.5° C.

At scheduled time intervals, 1 mL of samples were withdrawn from the outer compartments and replenished with the same volume of fresh medium. The RSV and CBD content in samples was measured using a UV-visible Spectrophotometer (U-3010, Hitachi, Japan) and calculated for amount of RSV and CBD released as a function of time.

### Example 25: In Vitro Release Protocols for Hybrid Excipients

Typical Polymer Adducts (i.e., Run #90 RSV or Run #108 CBD) were as mixed with individual insoluble, crosslinked, nanosponge-type compositions (i.e., Run #94 and Run #97), at 1:1 volume ratio and stirred overnight at RT to form the corresponding Hybrid Excipients to form multiple Hybrid Excipients. The mixing of a volume ratio of 1 Run #90 RSV or Run #108 CBD to 0.5 each of insoluble Run #94 and Run #97 and stirred overnight at RT. These complexes were evaluated by the dialysis technique described above.

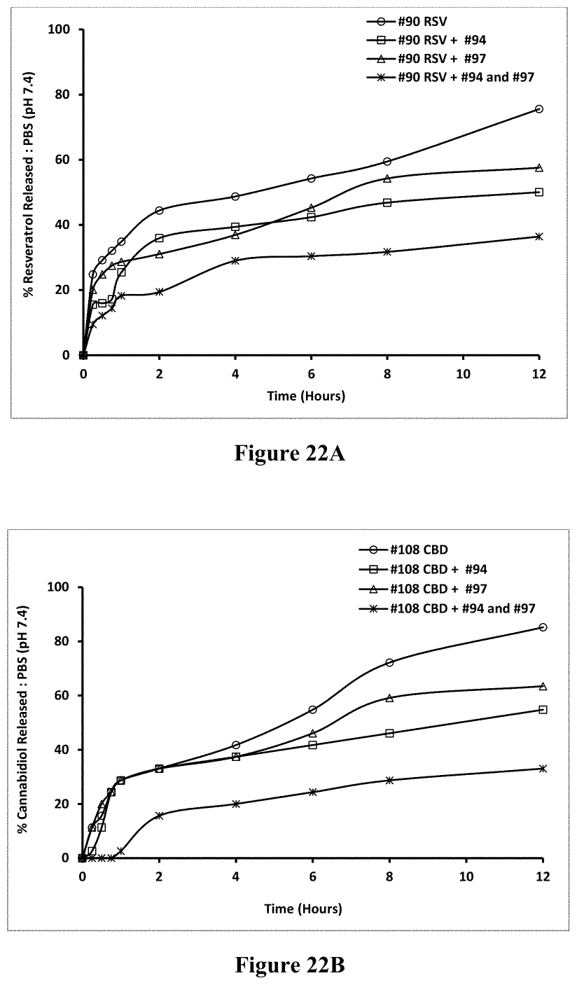

Almost 75.56% of RSV was released from non-hybrid Run #90 RSV in 12 hr. The release of RSV from Hybrid Excipient, Run #90 RSV+Run #94, Run #90 RSV+Run #97 were about 50.04% and 57.56%, respectively in 12 hr. Interestingly, only 36.42% of RSV was leached out from the combination of insoluble multiple Excipients (Run #94 and Run #97) in the same time period (FIG. 22A).

Similarly, 85.21% of CBD was released from non-hybrid Run #108 CBD in 12 hr. compared with 54.78% and 63.47% for Run #108 CBD+Run #94, Run #108 CBD+Run #97 (Hybrid Excipient). However, only 33.04% of CBD was leached out from the combination of insoluble multiple Excipients (Run #94 and Run #97) (FIG. 22B).

The slower drug release profile observed for RSV and CBD was possibly due to the participation of insoluble (Run #94 and Run #97) Excipients with RSV and CBD complexes which forms a viscous complex and allows release of the RSV and CBD in slower controlled manner (i.e., crosslinking effect).

### Example 26: In Vivo Human Oral Ingestion Studies of SupraPlex® Excipients (CBD/THC)

A highly documented “in house” CBD-SupraPlex® human oral ingestion study was performed to further evaluate the SupraPlex® Excipients in humans. This study involved two healthy male volunteers: Subject #1 was 33 years old, 5′10″, 190 lbs., native American (Chippewa Tribe) and an experienced CBD/THC consumer, and Subject #2 was 33 years old, 5′6″, 160 lbs., a naïve, and a THC/CBD non consumer from India (Hindu).

### Materials:

The CBD-SupraPlex® excipient samples were prepared under sanitary, GLP conditions. All CBD dose concentrations were confirmed by standard HPLC protocols.

### Testing:

The CBD-SupraPlex® excipient samples used for oral ingestion were administered sequentially in three, 10 mL ingestion portions as clear, transparent DI aqueous solutions. The 1^{st} portion was ingested in a sublingual mode and last two portions were ingested by normal swallowing. The esthetic taste features of CBD-SupraPlex® excipient (pH2), in DI water were: sour, citrus-lemon, (80% lemon-10% salt taste), slight terpene, slippery film like texture, but with no after taste. When used in ginger ale, it gave a very pleasant taste. At pH4, the sourness is eliminated to give a pleasant slight lemon taste.

The CBD-SupraPlex® Excipient (i.e., Run #181 above) complex in DI water produced a dramatic and observable “CBD effect” within 15-30 min. after ingestion at a dose level of about 1000m (about 1 mg) of CBD. This 1 mg dose level is 15×lower than the single 15 mg beverage dose level recommended by industry experts and 30×-50×lower than that recommended in the open CBD literature/industry (https://vitalleaf.com/how-much-cbd-should-i-take/).

Test 1: 10 mL containing 35.6 μg/mL of CBD in DI water, sublingual over 15 min.
Test 2: 15 min. after Test 1, 10 mL/35.6 μg/mL of CBD in DI water, normal swallow over 15 min.
Total CBD ingested=712 μg in 20 mL DI water over 30 min.
Test 3: 15 min. after Test 2, repeat of Test 2
Total CBD ingested=1068 μg in 30 mL DI water over 45 min.
Test 4: 2 days later, 40 mL/35.6 μg/mL of CBD in DI water, normal swallow before evening sleep.
Subject #1: starting blood pressure (bp) 112/69; within 15-30 min. after ingestion experienced tingling in fingers, heightened focus, higher energy, relaxed feeling, reduced anxiety, some thirst noted. Subject #1 could rarely experience more than 4 hr. normal sleeper night, but after Test 4 he took a 35.6 μg/mL of CBD in 40 mL DI water, sublingual before sleep 2 days after Test 4. When taken before bedtime, about 1 mg CBD dose level gave an excellent, sustained 7-8 hr. sleep cycle with a rested, clear-headed wake-up, no fogginess or after effect.
Subject #2: starting blood pressure (bp) 127/89 within 15-30 min. after ingestion experienced peaceful, relaxed, reduced anxiety, remained alert/coordinated, enhanced talking, laughing, higher energy movement, then some drowsiness after 1 hr. whereupon he lapsed into a sound nap for 10-15 min., followed by a clear-headed, lucid wakeup, no fogginess, highly alert, high energy, some thirst noted.

Because well-defined and differentiated dose level effects commensurate with experienced vs. non-experienced CBD consumers were observed, there does not appear to be any “placebo effect”.

This significantly lower dose requirement observed with SupraPlex® to produce this enhanced “CBD effect” provides strong evidence for the involvement of an unexpected, new solubilization/delivery mechanism when using the present CBD-SupraPlex® excipient formulations.

### Analysis of Oral Ingestion Results:

The documented low CBD dose levels (i.e., about 1 mg) observed for inducing the rapid onset (i.e., 15-30 min.) of a substantial “CBD effect” is attributed to an unprecedented new molecular level/nanoscale mechanism/concept for enhancing the bioavailability of insoluble APIs. It is apparent that hydrated, API based nano-assemblies of this invention are more effectively delivered in vivo to desired receptor sites; wherein, they may manifest more rapid and intense activity. These unprecedented new bioavailability properties are expected to be disruptive to the current CBD/THC industry; wherein, approximately 15-50× less CBD or THC is required to attain equivalent activity/efficacy based on traditional emulsion/micelle strategies versus using CBDSupraPlex® Excipient formulations.

Compared to current aqueous based CBD products, CBD-SupraPlex® excipient based formulations offer unprecedented, new opportunities for marketing more effective aqueous based CBD or THC products. These new excipient formulation options will impose no new burden on current operational protocols, while reducing CBD/THC concentrations (i.e., dose levels) required for equivalent activity. This will be expected to allow highly profitable commercialization of very effective CBD products requiring smaller facilities or lower hemp production levels.

The Polymer Adducts of this invention display and provide many advantages. Some of the most useful and surprising advantages are:

1. Solubility enhancement that achieved by:

a. having an aqueous SupraPlex® incubated with Guest molecule to saturate, followed by filtration and drying, giving a soluble powdered complex as the Polymer Adduct.

b. having an alcoholic aqueous SupraPlex® incubated with Guest molecule to saturate, followed by filtration and drying, giving a soluble powdered complex as the Polymer Adduct.

c. having aqueous SupraPlex® incubated with Guest molecule to saturate, followed by filtration and drying, giving an aqueous concentrate as the Polymer Adduct.

d. having alcoholic SupraPlex® incubated with Guest molecule to saturate, followed by filtration and drying, giving an alcoholic concentrate as the Polymer Adduct.

2. Stabilization of the Guest molecule by use of the Polymer Adduct to achieve protection from thermal degradation, protection from photodegradation, protection from acid or base degradation.
3. Providing ease in making compounding formulations by:

a. Polymer Adducts are used as powdered complexes that can be mixed with water, oil, or cream bases to prepare solutions, ointments, or creams.

b. Polymer Adducts as complex concentrates can be metered into mixtures to give desired concentrations of the complex in the formulation.

c. Polymer Adducts as powders or concentrates can be blended with beverages to give palatable formulations for oral administration.

4. Delivering/administration of Guest molecule by:

a. Oral administration—absorption of solutions through the linings of the mouth, stomach, and or intestines.

b. Transdermal—formulations of the complex in creams, gels, sera, or ointments that are applied topically.

c. Rectal—administration by means of creams, ointments, or suppositories

d. Ocular—drops of aqueous solutions for absorption through the eye.

e. Nasal—spray of aqueous solution for absorption by nasal lining.

f. Otologic—administration to the ear

g. Laryngeal/Tracheal—administration to the throat as sprays or solution

h. Respiratory—aerosols, inhalation of particulates, mists, or gasses

i. Vaginal—solutions, gels, suppositories, or enemas

j. Parenteral administration—various injection methods

5. Activity enhancement and bioavailability of the Guest molecule as mixtures of active compounds with SupraPlex® show more effect per unit of weight than the compound alone.
6. Stabilization of formulations of the Guest molecule in plastic, glass and metal containers.

Although the invention has been described with reference to its preferred embodiments, those of ordinary skill in the art may, upon reading and understanding this disclosure, appreciate changes and modifications which may be made which do not depart from the scope and spirit of the invention as described above or claimed hereafter. Accordingly, this description is to be construed as illustrative only and is for the purpose of teaching those skilled in the art the general manner of carrying out the invention.

What is claimed is:

1. A polymeric host compound comprising a tetrapolymeric compound of the formula
A_{w}BₓC_{y}D_{z} Formula (I)
wherein:
the polymer of Formula (I) is a cross-linked polymer, linear polymer, simple branched polymer, hyperbranched polymer or dendritic polymer; and
monomer A is at least one multifunctional carboxylic compound and monomers B, C and D are at least one poly(hydroxylic) alcohol that can be the same or different, wherein the molar ratio of A:B:C:D is (x+y+z)/w=0.05-4; or
monomers A and B are at least one multifunctional carboxylic compound that can be the same or different, and monomers C and D are at least one poly(hydroxylic) alcohol that can be the same or different, wherein the molar ratio of A:B:C:D is (y+z)/(w+x)=0.05-4; or
monomers A and C are at least one multifunctional carboxylic compound that can be the same or different, and monomers B and D are at least one poly(hydroxylic) alcohol that can be the same or different, wherein the molar ratio of A:B:C:D is (x+z)/(w+y)=0.05-4; or
monomers A, B and C are at least one multifunctional carboxylic compound that can be the same or different, and monomer D is at least one poly(hydroxylic) alcohol that can be the same or different, wherein the molar ratio of A:B:C:D is z/(w+x+y)=0.05-4; or
w and z must each be at least 1; and
x and y are independently either 0 or at least 1; and
provided that when x and y are both 0, then the polymer of Formula (I) is not crosslinked polymer.

2. The polymeric host compound of claim 1 wherein y is 0 comprising a terpolymeric compound of the formula
A_{w}BₓD_{z} Formula (II)
wherein:
the polymer of Formula (II) is a cross-linked polymer, linear polymer, simple branched polymer, hyperbranched polymer or dendritic polymer; and
monomer A is at least one multifunctional carboxylic compound, and monomers B and D are at least one poly(hydroxylic) alcohol that can be the same or different, wherein the molar ratio of A:B:D is (x+z)/w=0.5−4; or
monomers A and B are a poly(hydroxylic) alcohol that can be the same or different, and monomer D is a multifunctional carboxylic compound, wherein the molar ratio of A:B:D is z/(w+x)=0.05-4; and
w and z must both be at least 1; and
x can be 0 or at least 1.

3. The polymeric host compound of claim 1, wherein x and y are both 0 comprising a binary copolymer of the formula
A_{w}D_{z} Formula (III)
wherein:
the polymer of Formula (III) is a linear polymer, simple branched polymer, hyperbranched polymer or dendritic polymer; and
the monomer A is at least one multifunctional carboxylic compound; and
the monomer D is at least one poly(hydroxylic) alcohol; and
w and z are both at least 1; and
the molar ratio of A:D is z/w=0.05 to 4; and
provided that gel formation is minimized.

4. The polymeric host compound of claim 1 wherein the polymer is a linear polymer, hyperbranched polymer or dendritic polymer.

5. The polymeric host compound of claim 1 wherein the multifunctional carboxylic acid is citric, itaconic, aconitic, tartaric, malonic, malic, maleic, succinic, glutaric, adipic, pimelic, suberic, azelaic, tricarballylic, nitrilotriacetic, or ethylenediaminetetraacetic acid.

6. The polymeric host compound of claim 5 wherein the multifunctional carboxylic acid compound is citric acid.

7. The polymeric host compound of claim 1 wherein the poly(hydroxylic) alcohol is Cyclodextrin, glycerol, sorbitol, erythritol, threitol, glucose, glucosamine, tris(hydroxymethyl)aminomethane, propylene glycol, 1,3-propanediol, 1,4-butanediol, 1,5-pentanediol, 1,6-hexanediol, hydroxy terminated poly(ethylene glycols), hydroxy terminated poly(propylene glycols), xylitol, arabitol, ribitol, mannitol, inositol, pentaerythritol, monosaccharides, disaccharides, α-cyclodextrin, β-cyclodextrin, γ-cyclodextrin, sulfonated β-cyclodextrin, hydroxypropyl-β-cyclodextrin, methylated-β-cyclodextrin, polyethylene oxide, or polypropylene oxide.

8. The polymeric host compound of claim 7 wherein the poly(hydroxylic) alcohol is Cyclodextrin.

9. The polymeric host compound of claim 8 wherein the cyclodextrin has at least 2 appended carboxylate groups selected from carboxylic acid, ester, or activated ester.

10. The polymeric host compound of claim 1 wherein A is citric acid and D is a Cyclodextrin.

11. The polymeric host compound of claim 2 wherein A is citric acid, B is glycerol, and D is a Cyclodextrin.

12. The polymeric host compound of claim 1, wherein: A is citric acid; B is Cyclodextrin; C is glycerol; and D is another a Cyclodextrin other than B.

13. The polymeric host compound of claim 1 wherein the hyperbranched polymer is water soluble.

14. The polymeric host compound of claim 1 wherein the hyperbranched polymer is water insoluble.

15. The polymeric host compound of claim 1 wherein the dendritic polymer is a water soluble dendritic polymer wherein the monomers are citric acid and Cyclodextrin and the polyester layers are formed sequentially and the core is a poly(hydroxylic) alcohol.

16. The polymeric host compound of claim 1 wherein the polymeric host compound provides at least one or more of a pH stabilizing effect, photodegradation stabilization effect, plastic stabilization effect, and thermal stabilization effect.

17. A Polymeric Adduct comprising a polymeric host compound of claim 1 and at least one confined Guest molecule which provides water solubility enhancement from about 10-fold to about 1,000,000-fold.

18. The Polymeric Adduct of claim 17 wherein the polymeric host compound is water soluble and the Guest molecule is at least one of an API, OTC, AGI, VET, Cannabinoids or herbal extract.

19. The Polymeric Adduct of claim 18 formulated, using pharmaceutically-acceptable additive ingredients, as an oral means, foods, tablet, lozenge, capsule, syrup, sprays, or suspension; as a topical cream, powder, ointment, gel, paste, spray, foam, or aerosol; as an ophthalmic eye drops, ophthalmic ointment or gel; as a parenteral injection administered intramuscular, intravenous, or subcutaneous; as an inhalation treatment as an aerosol for the nose, nasal powder, or nebulizer; as an otic treatment by ear drops; as a rectal suppository or enema; or as a vaginal for humans or animals.

20. The Polymeric Adduct of claim 18 wherein the polymeric host compound is water insoluble and the Guest molecule is at least one of an API, OTC, AGI, VET, Cannabinoids, herbal extract, vitamin, food additive or supplement.

21. The Polymeric Adduct of claim 20 formulated, using pharmaceutically-acceptable additive ingredients, as an oral means, foods, tablet, lozenge, capsule, syrup, sprays, or suspension; as a topical cream, powder, ointment, gel, paste, spray, foam, or aerosol; as an ophthalmic eye drops, ophthalmic ointment or gel; as a parenteral injection administered intramuscular, intravenous, or subcutaneous; as an inhalation treatment as an aerosol for the nose, nasal powder, or nebulizer; as an otic treatment by ear drops; as a rectal suppository or enema; or as a vaginal for humans or animals.

22. The Polymeric Adduct of claim 18 wherein the Guest molecule is one or more synthetic compounds and/or natural extracts to increase the water solubility or bioavailability of the synthetic compounds and/or natural extracts.

23. The Polymeric Adduct of claim 18 wherein the polymeric host compound of claim 1 is further combined with a Cyclodextrin or a second different polymeric host compound of claim 1 as a Hybrid Excipient to increase the water solubility or bioavailability of the synthetic compounds and/or natural extracts.

24. The Polymeric Adduct of claim 18 wherein the Guest molecule is: an API, OTC, VET, AGI including but not limited to resveratrol; cannabidiol; or any compound bonded to or encapsulated or otherwise confined by a polymer of Formula (I), (II) or (III).

25. The Polymer Adduct of claim 18 for use as a stoichiometrically engineered, highly branched, nanoscale polymeric material that provide a technology for the development of products in life sciences, agriculture, AGI, pharmaceuticals, API, food-beverage industry, cannabinoids, pet food, veterinary, VET, dentistry, nutraceuticals, OTC, cosmetics, cosmeceuticals, aromatherapy or fragrances.

26. A method for increasing aqueous solubility of hydrophobic guest compounds comprising solubilizing the compounds by the use of water soluble polymeric host compounds of claim 1 in an aqueous solution, optionally where any residual reactants are present.

27. The method of claim 26 wherein a Polymeric Adduct is formed.

28. The method of claim 26 wherein the solubilizing compounds are Guest molecules in combination with reactants used to form compositions of claim 1.

29. The method of claim 28 wherein the Guest molecules are at least one of API, OTC, AGI, VET, Cannabinoids or herbal extracts.

30. A process for preparing a Polymer Adduct comprising reacting a solubilizing compound, a polymeric host compound and at least one Guest molecule in an aqueous solution at <140° C.

31. The process of claim 30 wherein the aqueous solution contains a solubilizing agent.

32. The polymeric host compound of claim 7 wherein the poly(hydroxylic) alcohol is tris(hydroxymethyl)aminomethane.

33. A method of tracing moieties and fluids in various systems by using a polymeric host compound of claim 32 comprising conducting tests, using in vitro applications, ex vivo applications, diagnostics applications or in vivo biological injection, topical applications or any customary pharmaceutical applications to such system, then by following the fluorescence in such system or test using visual or imaging apparatus in plants, animals and humans, by measuring fluorescence either qualitatively or quantitatively.
